(19) United States
(12) Patent Application Publication
(10) Pub. No.: US 2005/0112555 A1
(43) Pub. Date: May 26, 2005

(54) Cytidine deaminase activators, deoxycytidine deaminase activators, Vif antagonists, and methods of screening for molecules thereof

(76) Inventors: Harold C. Smith, Rochester, NY (US); Joseph E. Wedekind, Rochester, NY (US); Mark P. Sowden, Penfield, NY (US); Stephen Dewhurst, Rochester, NY (US); Baek Kim, Rochester, NY (US)

Correspondence Address: NEEDLE & ROSENBERG, P.C., SUITE 1000, 999 PEACHTREE STREET, ATLANTA, GA, 30309-3915, US

(21) Appl. No.: 10/934,090

(22) Filed: Sep. 3, 2004

Related U.S. Application Data
(60) Provisional application No. 60/499,953, filed on Sep. 3, 2003.

(51) Int. Cl. C12Q 1/70

(52) U.S. Cl. 435/5

(57) ABSTRACT

Disclosed are compounds that enhance RNA or DNA editing, as well as methods of using, identifying, and making such compounds. These compounds include Vif antagonists and cytidine deaminase inhibitors.

This invention claims priority to U.S. Provisional Application No. 60/499,953, filed Sep. 3, 2003.

This invention was made with government support under Grants RR15934, DK43738-08, F49620-01, and AI058789 from the National Institutes of Health. The government has certain rights in the invention.

### I. BACKGROUND OF THE INVENTION

HIV-1, a human lentivirus, is the causative agent of AIDS, which presently infects approximately 42 million persons worldwide with 1 million infected persons in. North America (http://www.unaids.org). The high mutation rate of HIV-1 has in the past made it impossible to develop therapies that retain their effectiveness. Current therapies for HIV infected patients target the production of new virus by antiviral agents that prevent replication of the viral RNA genomes into DNA prior to integration of the HIV DNA into chromosomal DNA or the disruption of the production or function of viral encoded proteins that are necessary for production of infectious viral particles. Antiviral agents that target viral replication have blunted the course of disease in patients already infected with HIV but these drugs have side effects due to toxicity and, while extending life for many patients, ultimately fail. Disruption of viral encoded protein production has not been as effective due largely to the high mutation rate of HIV and the consequent changing of viral protein into forms that retain function but no longer provide specific targets for the therapy. A combination of therapies together with better screening of blood supplies and blood products, improved public education and safe-sex practices has curbed the spread of disease only in developed countries but, even in these countries, preventative measures exhibit incomplete control over the spread of the virus.

Human white blood cells express a protein called CEM15, a cytidine deaminase, which can change the genetic code of the infecting AIDS viruses. These changes can render the virus incapable of producing an infection when they occur in critical genes encoding viral proteins and/or when they occur extensively throughout the HIV-1 genome. The AIDS virus, however, expresses a protein called Viral infectivity factor (Vif) that impairs the ability of CEM 15 to act on viral DNA. Interrupting deaminase functions in other systems such as the apolipoprotein B mRNA editing catalytic subunit 1 (APOBEC-1) and Activation Induced Deaminase (AID) systems have similar significance in the treatment of other diseases such as hypercholesterolemia and Hyper-IgM syndrome and certain forms of cancer (i.e., colorectal, APOBEC-1 and various leukemias and lymphomas). Thus, needed in the art is a means of enhancing deaminase function, or in the case of cancers, reducing or eliminating activity.

### II. SUMMARY OF THE INVENTION

In accordance with the purposes of this invention, as embodied and broadly described herein, this invention, in one aspect, relates to Vif antagonists. This invention also relates to cytidine deaminase activators, CEM15 activators, APOBEC-1 activators, and AID activators, and methods of identifying and making such agents.

In another aspect, this invention relates to deoxycytidine deaminase activators, ARP activators, and methods of identifying and making such activators.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### III. BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 shows the effect of introns on editing efficiency. (A) Diagram of the chimeric apoB expression constructs. The intron sequence (IVS) is derived from the adenovirus late leader sequence. Co-ordinates of the human apoB sequence are shown and the location of PCR amplimers indicated. X indicates the deleted 5′ splice donor or 3′ splice acceptor sequences. CMV is cytomegalovirus. (B) Poisoned-primer-extension assays of amplified apoB RNAs. Pre-mRNA and mRNA were amplified with the MS1/MS2 or SP6/T7 amplimers respectively. Editing efficiencies, an average for triplicate transfections, for each RNA are shown beneath. Percent editing efficiency was determined as the number of counts in edited apoB mRNA (UAA) divided by the sum of counts in UAA plus those in unedited apoB mRNA (CAA) and multiplied by 100.

FIG. 2 shows the effect of intron proximity on editing efficiency. (A) Diagram of the chimeric apoB expression constructs. IVS-(IVSΔ3′5′)-apoB and IVS-(IVSΔ3′5′)₂-apoB were created by the insertion of one or two copies respectively of the IVSΔ3′5′ intron cassette into IVS-apoB. Human apoB co-ordinates and amplimer annealing sites are indicated (FIG. 1). (B) Poisoned-primer-extension assays of amplified apoB RNAs. Pre-mRNA and mRNA were amplified with the MS7/MS2 or SP6/T7 amplimers respectively. Editing efficiencies, an average for duplicate transfections, for each RNA are shown beneath.

FIG. 3 shows that the editing sites within introns are poorly utilized. (A) Diagram of the chimeric apoB expression constructs. The apoB editing cassette was inserted as a PCR product into a uniqute HindIII site 5′ of the polypyrimidine tract in IVS-apoB and IVS-A3′5′apoB (FIG. 1). Amplimer annealing sites are indicated. (B) Poisoned primer extension assays of amplified apoB RNAs. Unspliced pre-mRNA and intron containing RNA were amplified with the Ex1/Ex2 or MS D5/MS D6 amplimers respectively. Editing efficiencies, an average for duplicate transfections, for each RNA are shown beneath.

FIG. 4 shows that editing is regulated by RNA splicing. (A) Diagram of the modified CAT reporter construct (CMV128) used in the Rev complementation assay; a gift from Dr Thomas J. Hope of the Salk Institute. The splice donor (SD), splice acceptor (SA), RRE, intron and 3′ long tandem repeat (LTR) are from the HIV-1 genome. CMV128 was modified by insertion of the apoB editing cassette as a PCR product into the BamHI site 3′ of the CAT gene. Amplimer annealing sites are indicated. (B) McArdle cell CAT activity in the absence (Vector) or presence of the Rev transactivator. Values are averages for duplicate experiments. CMVCAT was an assay control transfection. (C) Poisoned-primer-extension assays of amplified apoB RNAs. ‘Intron and exon RNA’ was amplified using the EF/MS2 amplimers. Editing efficiencies for each RNA are shown beneath. Promiscuous editing is indicated by ‘1’.

FIG. 5 shows representative members of the APOBEC-1 related family of cytidine deaminases including CEM15. Also are APOBEC-1 complementation factor (ACF) and viral infectivity factor (Vif). The catalytic domain of APOBEC-1 is characterized by a ZDD with three zinc ligands (either His or Cys), a glutamic acid, a proline residue and a conserved primary sequence spacing (Mian, I. S., et al., (1998) J Comput Biol. 5:57-72.). The ZDD of other deaminases and APOBEC-1 related proteins is shown for comparison along with a consensus ZDD. The indicated residues in the catalytic site of APOBEC-1 bind AU-rich RNA with weak affinity. The leucine rich region (LRR) of APOBEC-1 has been implicated in APOBEC-1 dimerization and shown to be required for editing (Lau, P. P., et al., (1994) Proc Natl Acad Sci USA. 91:8522-6; Oka, K., et al., (1997) J Biol Chem. 272:1456-60.) but structural modeling suggests that LRR forms the hydrophobic core of the protein monomer (Navaratnam, N., et al., (1998) J Mol Biol. 275:695-714.). ACF complements APOBEC-1 through its APOBEC-1 and RNA bindings activities. The RNA recognition motifs (RRM)s are required for mooring sequence-specific RNA binding and these domains plus sequence flanking them are required for APOBEC-1 interaction and complementation (Blanc, V., et al., (2001) J Biol Chem. 276:46386-93; Mehta, A., et al., (2002) RNA. 8:69-82.) APOBEC-1 complementation activity minimally depends on ACF binding to both APOBEC-1 and mooring sequence RNA. A broad APOBEC-1 complementation region is indicated that is inclusive of all regions implicated in this activity (Blanc, V., et al., (2001) J Biol Chem. 276:46386-93; Mehta, A., et al., (2002) RNA. 8:69-82.) Experiments have shown the N-terminal half of Vif is necessary for viral infectivity (Henzler, T. 2001). However, reports have demonstrated that residues in the C-terminus (amino acids 151-164) are essential for infectivity (Yang, S. et al. 2001) and that multimerization of Vif through the motif PPLP within this region was essential for infectivity. Peptides capable of binding to this domain of Vif blocked Vif-Vif interactions and Vif-Hck interactions in vitro and suppressed viral infectivity in cell-based assay systems. Residues in the N-terminus of Vif are essential for RNA binding and packing of Vif within the virion (Zhang et al. 2000; Khan et al. 2001; Lake et al. 2003).

FIG. 6 shows schematic depictions of the cytidine deaminase (CDA) polypeptide fold and structure-based alignments of APOBEC-1 with respect to its related proteins (ARPs). FIG. 6a depicts a gene duplication model for cytidine deaminases. CDD1 belongs to the tetrameric class of cytidine deaminases with a quaternary fold nearly identical to that of the tetrameric cytidine deaminase from B. subtilis (Johansson, E., et al., (2002) Biochemistry. 41:2563-70.). Such tetrameric enzymes exhibit the classical αββαβαββ topology of the Zinc Dependent Deaminase Domain (ZDD) observed first in the Catalytic Domain (CD) of the dimeric enzyme from E. coli (Betts, L., et al., (1994) J Mol Biol. 235:635-56). According to the gene duplication model, an ancestral CDD 1-like monomer (upper left ribbon) duplicated and fused to produce a bipartite monomer. Over time a C-terminal Pseudo-Catalytic Domain (PCD) arose that lost substrate and Zn²⁺ binding abilities (upper right ribbon). The model holds that the interdomain CD-PCD junction is joined via flexible linker that features conserved Gly residues necessary for catalytic activity on large polymeric DNA or RNA substrates. The function of the PCD is to stabilize the hydrophobic monomer core and to engage in auxiliary factor binding. The loss of PCD helix α1 can provide a hydrophobic surface were auxiliary factors bind to facilitate substrate recognition thereby regulating catalysis. The enzymes remain oligomeric because each active site comprises multiple polypeptide chains. Modem representatives of the chimeric CDA fold include the enzyme from E. coli, as well as APOBEC-1 and AID. Other ARPs such as APOBEC-3G (CEM15) may have arisen through a second gene duplication to produce a pseudo-homodimer on a single polypeptide chain (lower ribbon); structural properties of the connector polypeptide are unknown. Signature sequences compiled from strict structure-based alignments (upper) are shown below respective ribbon diagrams, where X represents any amino acid. Linker regions (lines) and the location of Zn²⁺ binding (spheres) are depicted. Although experimental evidence suggests APOBEC-3B has reduced Zn²⁺ binding and exists as a dimer (Jarmuz, A., et al., (2002) Genomics, 79:285-96), modeling studies suggest it will bind Zn²⁺ (as shown in Wedekind et al. Trends Genet, 19(4):207-16, 2003) and may function as a monomer. Inset spheres represent proper (222) CDD1-like quaternary structure symmetry whereas APOBEC-1-like enzymes exhibit pseudo-222 symmetry relating CD and PCD subunits; in the latter enzyme a proper dyad axis relates the polypeptide chains. Finally, APOBEC-3G can fold as a monomer from a single polypeptide chain with each CD and PCD (differently colored spheres in lower left inset box) related by improper 222 symmetry with no strict axes of symmetry. FIG. 6b depicts the structure based sequence alignment for ARPs. Sequences from human APOBEC-1, AID, and APOBEC-3G were aligned based upon a main-chain alpha-carbon least-squares superposition of the known cytidine deaminase three dimensional crystal structures from E. coli, B. subtilis and S. cerevisiae (FIG. 6c). Amino acid sequence alignments were optimized to minimize gaps in major secondary structure elements, which are depicted as tubes (α-helices) and arrows (β-strands) in FIG. 6b. Additionally, loops, turns, and insertions of FIG. 6b are marked L and T and i, respectively. L-C1 and L-C2 represent distinct loop structures in the dimeric versus tetrameric cytidine deaminases. Sections of basic residues that overlap the bipartite NLS of APOBEC-1 are marked BP-1 and BP-2. FIG. 6d depicts a schematic diagram of the domain structure observed in APOBEC-1 and related ARPs based upon computer-based sequence alignments using the ZDD signature sequence shown in the lower panel of FIG. 6a.

FIG. 7 shows the relation of CEM15 amino acid sequence to APOBEC-1 and other APOBEC-1 Related Proteins (ARPs) by use of standard computational methods based upon amino acid similarity or identity. Amino acid sequence alignments illustrate conservation of Zn⁺ ligands and key catalytic residues essential to the mechanism of hydrolytic deamination by cytidine deaminases (CDA). Collectively, these amino acids form a signature zinc-dependent deaminase domain (ZDD), present in: (i) APOBEC-1, which mediates C to U editing of apoB mRNA, (ii) the Activation Induced Deaminase (AID), which mediates Somatic Hypermutation (SHM) and Class Switch Recombination (CSR), and (iii) CEM15, which blocks HIV-1 viral infectivity.

FIG. 8 shows a schematic ribbon diagram depicting a three-dimensional model of APOBEC-1 derived from comparative modeling by the method of satisfying spatial restraints. Structure-based homology modeling has provided insight into the fold of APOBEC-1, and has been corroborated by protein engineering, site-directed mutagenesis, and functional analyses. The current model for APOBEC-1 predicts a two domain structure comprising a catalytic domain (CD) and a pseudo-catalytic domain (PCD) joined by a central linker, which folds over the active site (green segment). The linker sequence is conserved among ARPs (FIG. 6b), and linker sequence composition and polypeptide chain length are essential for efficient RNA editing by APOBEC-1. The APOBEC-1 model also provides a rationale for losses in editing due to surface point mutations, such as F156L, located 25 Å from the active site. This aromatic to branched-chain hydrophobic change appears to have no influence on the stability of the enzyme core, but can be involved in auxiliary factor binding required for RNA binding. Similarly, a series of basic residues at BP2 (FIG. 6b) are close to the active site, and can be responsible for RNA binding. Mutagenesis of all basic residues within the respective bp-clusters abolishes editing activity (Teng, 1999, J. Lipid Res. 40:623). The structural template of the APOBEC-1 model is derived from the spatial constraints derived from a superposition of three high resolution CDA crystal structures that exhibit a nearly identical αβ₂αβαβ₂ fold despite modest sequence identity (˜24% FIG. 6c); fold conservation also exists at the oligomeric level, since each enzyme exhibits either proper 222 or pseudo˜222 symmetry. Similarities in the Zn²⁺ dependent deaminase mechanism, as well as structural similarities among the known CDAs of pyrimidine metabolism show that the ARP fold is evolutionarily conserved among dimeric CDAs that act on RNA and DNA. Similarly, it is likely that CEM15 (APOBEC-3G) evolved from an APOBEC-1-like precursor by gene duplication. Thus, the CEM15 structure comprises two active sites per polypeptide chain with the topology CD1-PCD1-connector-CD2-PCD2 (FIG. 6a).

FIG. 9 shows a structural model for CEM15. The use of comparative modeling by the method of satisfied spatial restraints has allowed the calculation of a CEM15 three-dimensional model including all atoms of the 384 amino sequence. Spatial restraints for the template were derived from the atomic coordinates of three known CDA crystal structures including a bona fide RNA editing enzyme from yeast Cdd1, which is capable of deaminating free nucleosides as well as polymeric RNA substrates, such as reporter apoB mRNA. The known CDA crystal structures represent both dimeric and tetrameric quaternary folds (FIG. 6a), which allows an accurate model to be prepared using multiple structural restraints. Further insight into the CEM15 structure has also been attained by analogy to modeling and functional results obtained from APOBEC-1. A comparative model of CEM15 was calculated by use of the program ‘Modeller’ and subsequently checked by the program suites PROCHECK and the Verify3D server. The model was energy minimized using simulated annealing and molecular dynamics methods. No restraints were placed on secondary elements, except those derived from the triple CDA structure alignment. The position of the UMP nucleotide was incorporated based upon spatial restraints derived from known crystal structures. Zn²⁺ atoms were restrained using reasonable coordination geometry derived from the known CDAs. The resulting model demonstrated that the 384 amino acid sequence of CEM15 can be accommodated by a dimeric CDA quaternary fold (analogous to the E. coli CDA or APOBEC-1 with 2×236 amino acids).

FIG. 10 shows possible CEM15 oligomers. The number of possible CEM15 quaternary structures is limited and the actual oligomeric state can be evaluated by gel filtration chromatography, or through site directed mutagenesis that evaluates the requirement of single or dual CD domains in CEM15 activity. For example, possible dimeric CEM15 structures (FIGS. 10c and 10d) predict mutually exclusive intermolecular contacts with the distinguishing feature that the interaction observed in FIG. 10c is such that each CD pairs with itself, and similarly for each PCD. In contrast, every domain in FIG. 10d falls in a unique environment (i.e. no CD or PCD pairs with itself). A variety of truncation mutations address the question of whether or not a dimer of the form in head-to-head or head-to-tail exists in solution (FIGS. 10c versus 10d).

FIG. 11 shows HA-tagged CEM15 in 293T cells. Stable, HA-tagged CEM15 expressing 293T cell lines were selected with puromycin and verified by western blotting with a HA specific monoclonal antibody. The addition of the HA epitope tag has no effect on the ability of CEM15 to suppress infectivity. Isogenic HIV-1 pro-viral DNAs are packaged into pseudotyped lentiviral particles by co-transfection with a plasmid encoding the VSV G-protein into 293T cells that lack endogenous CEM15 (−) or expressed wild type CEM15 (+).

FIG. 12 shows the results of the assay described in Example 4, indicating that the expression of CEM15 in 293T cells resulted in at least a 100-fold decrease in Vif-viral infectivity compared to particles generated in parental 293T cells. The low level of GFP expression from vif−, CEM15+ particles is indistinguishable from background fluorescence in control cells.

FIG. 13 shows poisoned primer extension assays and Western analysis for Cdd1 mutants and chimeric proteins. In the context of late log phase growth in yeast with galactose feeding, overexpressed Cdd1 is capable of C to U specific editing of reporter apoB mRNA at site C₆₆₆₆ at a level of 6.7%, which is ˜10× times greater than the negative control (FIG. 13, empty vector—compare lanes 1 and 2). In contrast, the CDA from E. coli (equivalent to PDB entry 1AF2) is incapable of editing on the reporter substrate (FIG. 13, lane 3). Similarly, the active site mutants E61A and G137A abolish detectable Cdd1 activity (FIG. 13, lanes 4 and 5). Likewise, the addition of the E. coli linker sequence (FIG. 13, lane 6) impairs editing function as well. In a series of chimeric constructs in which the Cdd1 tetramer was converted into a molecular dimer, the chimeric molecule appears functional, as long as an amino acid linker of 7-8 amino acids is used to join the respective Cdd1 subunits (FIG. 13, Right Panel lanes 1-4). However, when the longer E. coli linker is used to join Cdd1 monomers, there is no detectable activity on the reporter substrate, although the chimeric protein is expressed (FIG. 13, Western blot). Paradoxically, when conserved Gly residues of the APOBEC-1 linker (130 and 138) are mutated to Ala, the chimeric enzyme is still active (FIG. 13, lanes 3 and 4 of right panel), although this result is consistent with the observation that APOBEC-3G can utilize a non-Gly residue at this position (FIG. 6b).

FIG. 14 shows reduced production of pseudotyped HIV-1 viral particles by cells expressing CEM15 or DM. p24 concentration (pg/ml) normalized to % GFP containing cells (as a measure of transfection efficiency) for 293T cells stably expressing pIRES-P vector (n=6), CEM15 (n=6) and DM (n=5), following transfection with wild-type (Vif+) or ΔVif proviral DNA plasmids (black and white bars, respectively). Error bars represent standard deviation calculated from n for each cell line.

FIG. 15 shows CEM15 suppresses HIV-1 protein abundance. 293T cell lines stably expressing (A) CEM15, (B) DM, and (C) control pIRES-P vector were transiently transfected with proviral HIV-1 plasmids (containing either wild-type Vif (+) or ΔVif (−)). Total cell lysates were prepared at 24, 48, and 72 hours post-transfection, separated by SDS-PAGE and analyzed by immunoblot assay using antibodies reactive with HA (HA-tagged CEM15 and DM), Vif, p24, RT, β-actin, Vpr, or Tat (as denoted on the left). The molecular weight (kDa) of the indicated protein species is given to the right.

FIG. 16 shows CEM15 suppresses HIV-1 viral RNA abundance. (A) Location of Gag-Pol junction and protease region of HIV-1 genomic RNA corresponding to the GP-RNA probe used for RNA binding and northern blot analysis. (B) UV crosslinking of increasing concentration of recombinant CEM15 protein (1, 2 and 4 μg protein) to 20 fmol radiolabeled GP-RNA and apoB RNA. (C) Poly A+ RNA abundance for Gag-Pol transcripts in 293T-CEM15 at 24, 48, and 72 hours and DM cells at 48 hours post-transfection with Vif+ (black) and ΔVif (white) proviral DNA. Results are expressed as the ratio of viral RNA (GP-RNA region) to endogenous cellular RNA (adenovirus EIA) determined through phosphorimager scanning densitometry analysis of northern blots.

### IV. DETAILED DESCRIPTION

The invention provides compounds that enhance RNA or DNA editing, as well as methods of using, identifying, and making such compounds. The compounds are useful in preventing or treating a variety of diseases, including viral infections. Described herein are cytosine deaminase activators and antagonists of compounds, like viral infectivity factor (vif), that interfere with deaminases.

### A. RNA AND DNA EDITING

There are several examples of cellular and viral mRNA editing in mammalian cells. (Grosjean and Benne (1998); Smith et al. (1997) RNA 3: 1105-23). Two examples of such editing mechanisms are the adenosine to inosine and cytidine to uridine conversions. (Grosjean and Benne (1998); Smith et al. (1996) Trends in Genetics 12:418-24; Krough et al. (1994) J. Mol. Biol. 235:1501-31). Editing can also occur on both RNA and on DNA, and typically these functions are performed by different types of deaminases.

A to I editing involves a family of adenosine deaminases active on RNA (ADARs). ADARs typically have two or more double stranded RNA binding motifs (DRBM) in addition to a catalytic domain whose tertiary structure positions a histidine and two cysteines for zinc ion coordination and a glutamic acid residue as a proton donor. The catalytic domain is conserved at the level of secondary and tertiary structure among ADARs, cytidine nucleoside/nucleotide deaminases and CDARs but differs markedly from that found in adenosine nucleoside/nucleotide deaminases (Higuchi et al (1993) Cell 75:1361-70). ADAR editing sites are found predominantly in exons and are characterized by RNA secondary structure encompassing the adenosine(s) to be edited. In human exon A to I editing, RNA secondary structure is formed between the exon and a 3′ proximal sequence with the downstream intron (Grosjean and Benne (1998); Smith et al. (1997) RNA 3: 1105-23; Smith et al. (1996) Trends in Genetics 12:418-24; Maas et al (1996) J. Biol. Chem. 271:12221-26; Reuter et al. (1999) Nature 399:75-80; O'Connell (1997) Current Biol. 7:R437-38). Consequently, A to I editing occurs prior to pre-mRNA splicing in the nucleus. The resultant inosine base pairs with cytosine and codons that have been edited, effectively have an A to G change. ADAR mRNA substrates frequently contain multiple A to I editing sites and each site is selectively edited by an ADAR, such as ADAR1 or ADAR2. ADARs typically function autonomously in editing mRNAs. ADARs bind secondary structure at the editing site through their double stranded RNA binding motifs or DRBMs and perform hydrolytic deamination of adenosine through their catalytic domain.

1. APOBEC-1

One example of a Cytosine Deaminase Active on RNA (CDAR) is APOBEC-1 (apolipoprotein B mRNA editing catalytic subunit 1) (accession #NM₁₃ 005889) encoded on human chromosome 12. (Grosjean and Benne (1998); Lau et al. (1994) PNAS 91:8522-26; Teng et al (1993) Science 260:1816-19). APOBEC-1 edits apoB mRNA primarily at nucleotide 6666 (C6666) and to a lesser extent at C8702 (Powell et al. (1987) Cell 50:831-40; Chen et al. (1987) Science 238: 363-366; Smith (1993) Seminars in Cell Biology 4:267-78) in a zinc dependent fashion (Smith et al. (1997) RNA 3:1105-1123). This editing creates an in-frame translation stop codon, UAA, from a glutamine codon, CAA at position C6666 (Grosjean and Benne (1998); Powell et al. (1987) Cell 50:831-840; Chen et al. (1987) Science 238:363-66). The biomedical significance of apoB mRNA editing is that it results in increased production and secretion of B48 containing very low density lipoproteins and correspondingly, a decrease in the abundance of the atherogenic apoB100 containing low density lipoproteins in serum (Davidson et al. (1988) JBC 262:13482-85; Baum et al. (1990) JBC 265:19263-70; Wu et al. (1990) JBC 265:12312-12316; Harris and Smith (1992) Biochem. Biophys. Res. Commun. 183:899-903; Inui et al. (1994) J. Lipid Res. 35:1477-89;Funahashi et al (1995) J. Lipid Res. 36:414-428; Giannoni et al. J. Lipid Res. 36:1664-75; Lau et al. (1995) J. Lipid Res. 36: 2069-78; Phung et al. (1996) Metabolism 45:1056-58; Van Mater et al. (1998)Biochem. Biophys. Res. Commun. 252:334-39; von Wronski et al. (1998) Metab. Clin.Exp. 7:869-73).

In APOBEC-1 gene knockout mice, apoB mRNA was unedited, demonstrating that no other CDAR is expressed which can use apoB mRNA as a substrate (Nakamuta et al. (1996) JBC 271:25981-88;Morrison et al. (1996) PNAS 271:25981-88; Hirano et al. (1996) J. Biol. Chem. 271:9887-90; Yamanaka et al. (1997) Genes Dev. 11:321-33; Yamanaka et al. (1995) PNAS 92:9493-87; Sowden et al. (1998) Nucl. Acids Res. 26:1644-1652). ApoB is translated from a 14 kb mRNA that is transcribed from a single copy gene located on human chromosome 2 (Scott (1989) J. Mol. Med. 6:65-80). ApoB protein is a non-exchangeable structural component of chylomicrons and of very low density (VLDL) and low density (LDL) lipoprotein particles. APOBEC-1 editing of apoB mRNA determines whether a small (apoB48) or a large (apoB100) variant of apoB lipoprotein is expressed (Grosjean and Benne (1998); Powell et al. (1987) Cell 50:831-840; Chen et al. (1987) Science 238:363-66; Scott (1989) J. Mol. Med. 6:63-80; Greeve et al (1993) J. Lipid Res. 34:1367-83).

In contrast to A to I editing, RNA secondary structure does not appear to be required for apoB RNA editing. Instead, apoB mRNA editing requires an 11 nucleotide motif known as the mooring sequence. Placement of the mooring sequence 4-8 nucleotides 3′ of a cytidine within reporter RNAs is frequently sufficient for that RNA to support editing (Smith (1993) Seminars in Cell Biol. 4:267-78; Sowden et al. (1998) Nucl. Acids Res. 26:1644-1652; Backus and Smith (1992) Nucl. Acids Res. 22:6007-14; Backus and Smith (1991) Nucl. Acids Res. 19:6781-86; Backus and Smith (1994) Biochim. Biophys. Acta 1217:65-73; Backus et al. (1994) Biochim. Biophys. Acta 1219:1-14; Sowden et al. (1996) RNA 2:274-88). The mooring sequence is left intact in edited mRNA and therefore its occurrence downstream of a cytidine is predictive of an editing site.

APOBEC-1 relies on auxiliary proteins for RNA recognition (Grosjean and Benne (1998); Teng et al. (1993) Science 260:1816-19; Sowden et al (1998) Nucl. Acids Res. 26:1644-52; Inui et al. (1994) J. Lipid Res. 35:1477-89; Dance et al. (2001) Nucl. Acids Res. 29:1772-80). APOBEC-1 only has weak RNA binding activity of low specificity (Anant et al. (1995) JBC 270:14768-75; MacGinnitie et al. (1995) JBC 270:14768-75). To edit apoB mRNA, APOBEC-1 requires a mooring sequence-specific, RNA binding protein that binds apoB mRNA and to which APOBEC-1 can bind and orient itself to C6666. Under defined in vitro conditions, apoB RNA, recombinant APOBEC-1 and proteins known as ACF/ASP (APOBEC-1 Complementing Factor/APOBEC-1 Stimulating Protein) were all that was required for editing activity and are therefore considered as the minimal editing complex or editosome (Mehta et al. (2000) Mol. Cell Biol. 20:1846-54; Lellek et al. (2000) JBC 275:19848-56).

ACF was isolated and cloned using biochemical fractionation and yeast two hybrid genetic selection (Mehta et al. (2000) Mol. Cell Biol. 20:1846-54; Lellek et al. (2000) JBC 275:19848-56). Overexpression of 6His-tagged APOBEC-1 in mammalian cells enabled the intracellular assembled editosome to be affinity purified (Yang et al. (1997) JBC 272:27700-06). These studies demonstrated that ACF associated with APOBEC-1 through 1M NaCl resistant interactions and that three other RNA binding proteins (100 kDa, 55 kDa and 44 kDa) with affinity for the mooring sequence co-purified with the editosome (Yang et al. (1997) JBC 272:27700-06). P100 and p55 were both mooring sequence selective RNA binding proteins but p44 was a general RNA binding protein. Additional studies utilizing yeast two hybrid analyses using APOBEC-1 affinity and antibodies developed against the editosome and ACF have demonstrated proteins such as hnRNP ABBP1 (Lau et al. (1997) JBC 272:1452-55), the alternative splicing factor KSRP (Lellek et al. (2000) JBC 275:19848-56) and alpha13 (αI3) serum proteinase inhibitor as positive modulators of editing activity (Schock et al, (1996) PNAS 93:1097-1102) and hnRNP protein C (Greeve et al. (1998) Biol. Chem. 379:1063-73) and GRY-RBP (Blanc et al. (2001) JBC 276: 10272-83; Lau et al. (2001) Biochem. Biophys. Res. Commun. 282:977-83) as negative modulators of apoB mRNA editing.

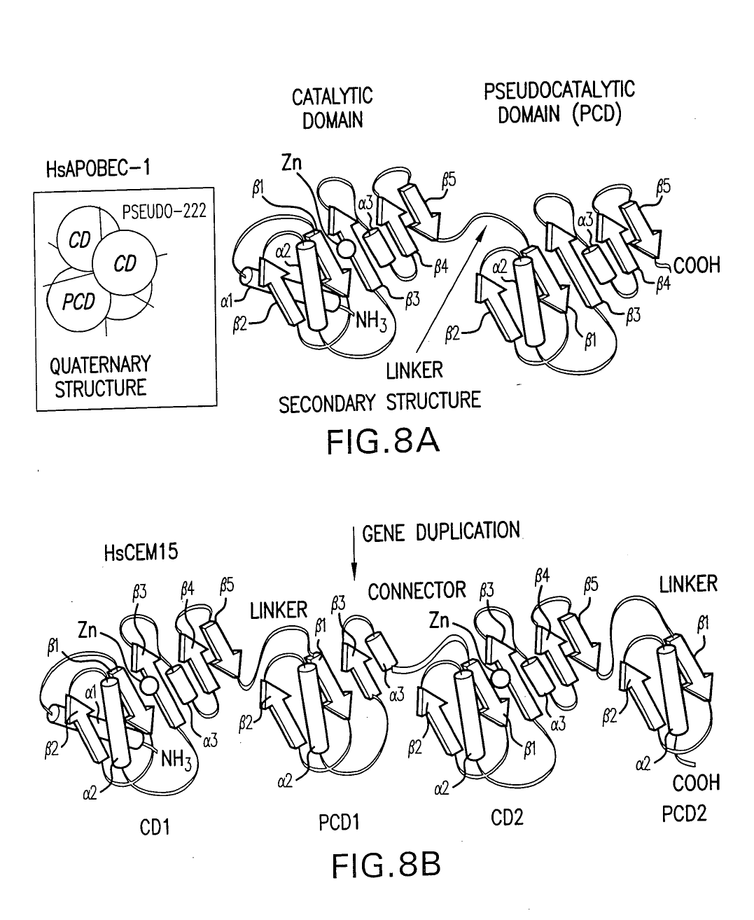

Structure-based homology modeling has provided insight into the fold of APOBEC-1 (FIG. 8; Wedekind et al. Trends Genet, 19(4):207-16, 2003), and the modeling of APOBEC-1 has been corroborated by protein engineering, site-directed mutagenesis, and functional analyses. The current model for APOBEC-1 is a two domain structure comprising a catalytic domain (CD) and a pseudo-catalytic domain (PCD) joined by a central linker, which folds over the active site (FIG. 8). The linker sequence is conserved among ARPs, and sequence identity and length are essential for efficient RNA editing by APOBEC-1. The APOBEC-1 model also provides a rationale for losses in editing due to surface point mutations, such as F156L (Navaratnam et al. Cell 81(2)187-95), located 25 Å from the active site. Such a change can influence auxiliary factor binding. Other mutations such as K33A/K34A abolish activity (Teng et al. J Lipid Res, 40(4) 623-35, 1999).

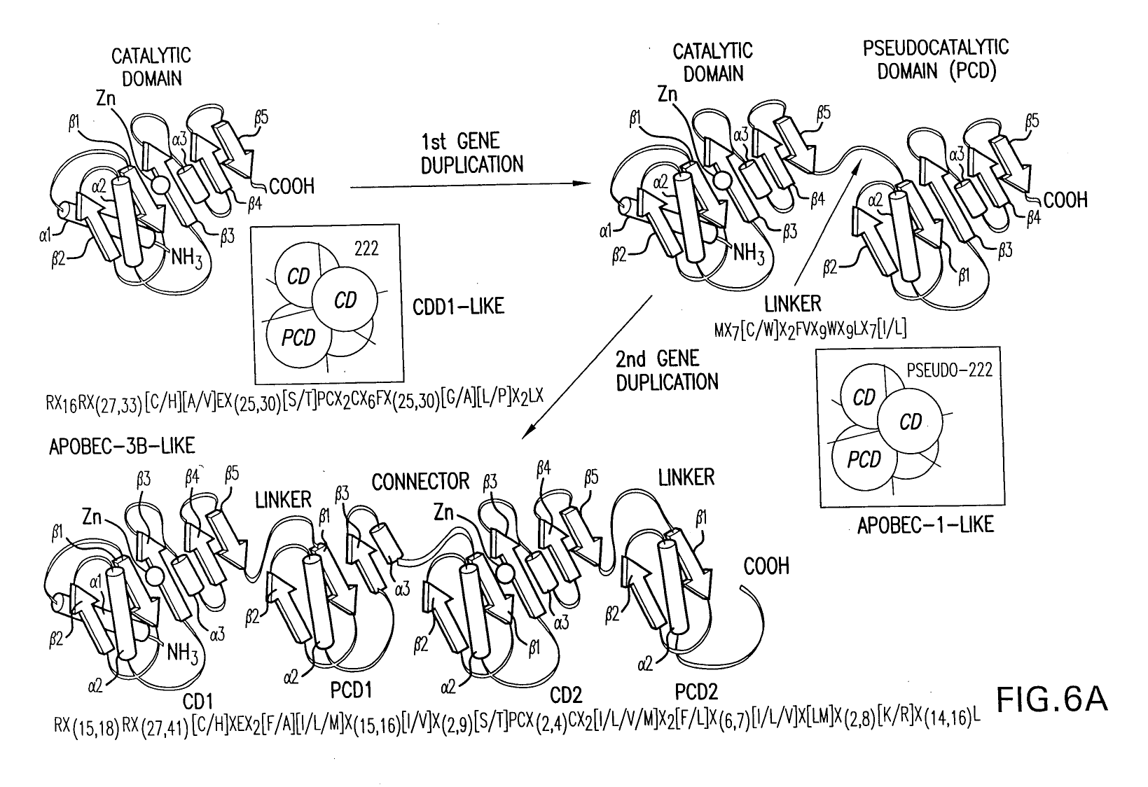

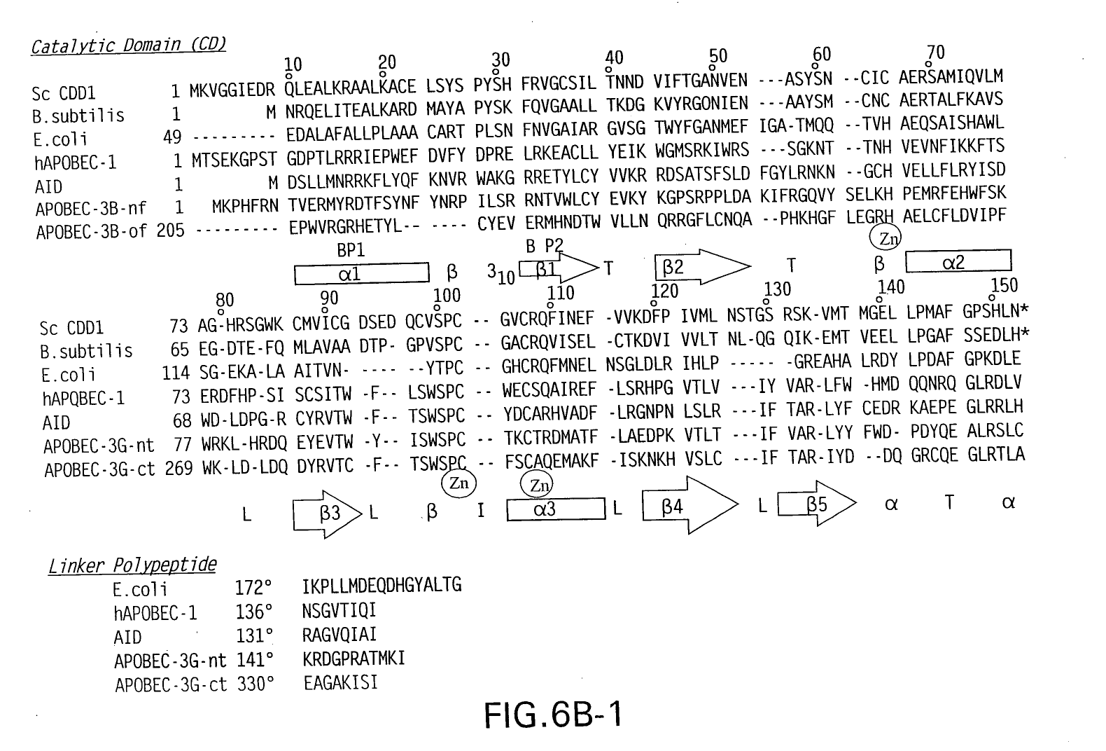

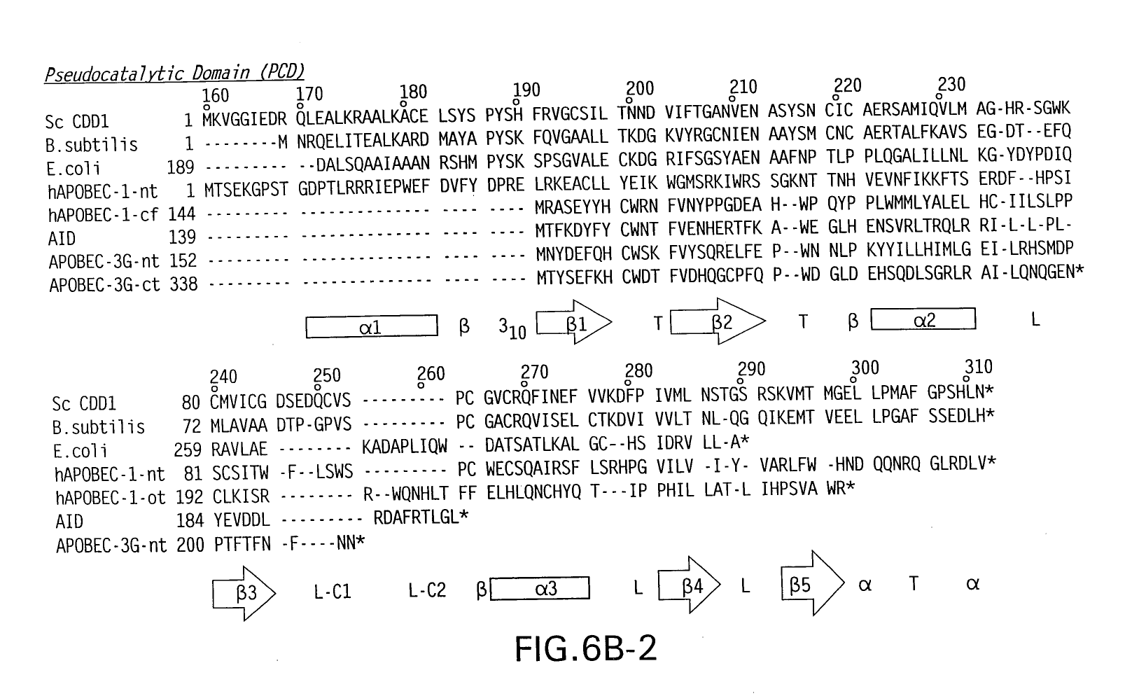

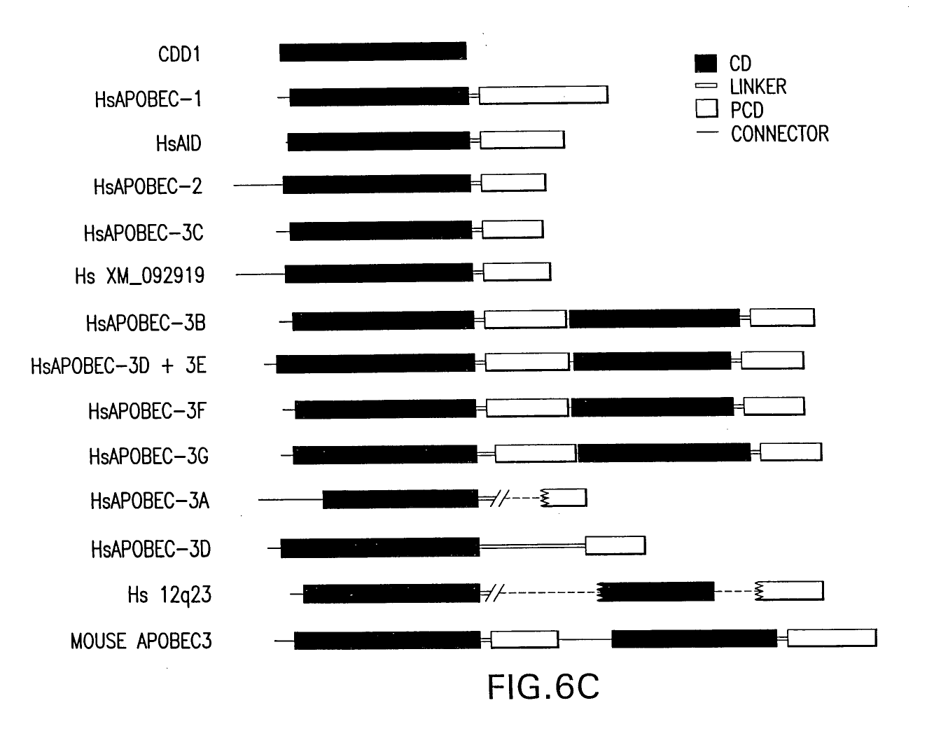

Other mutations such as K33A/K34A abolish activity (Teng et al. J Lipid Res, 40(4) 623-35, 1999). These basic residues are a feature of all ARP family members, including CDD1. In the model the latter basic residues are close to the active site, and can be responsible for RNA binding. The spatial restraints and fidelity of the APOBEC-1 model is derived from superposition of three high resolution CDA crystal structures (Betts et al. J Mol Biol 235(2):635-56, 1994; Johansson et al. Biochemistry 41(8): p. 2563-70, 2000) that exhibit a nearly identical αβ₂αβαβ₂ fold despite modest sequence identity (˜24%); fold conservation also exists at the oligomeric level, since each enzyme exhibits proper or nearly proper 222 symmetry (FIGS. 6a and 6c).

Structural homology is derived from the fact that dimeric CDAs arose from gene duplication of a CD precursor (Betts et al. J Mol Biol 235(2):635-56, 1994; Johansson et al. Biochemistry 41(8): p. 2563-70, 2000) producing a PCD, which although catalytically inactive, forms an inextricable part of the core protein fold and the enzyme active site. Pairwise superpositions of 75 backbone atoms from the yeast CDD1 crystal structure with comparable atoms from those CDA structures of E. coli and B. subtilis results in rmsds of 1.22 Å and 0.77 Å, respectively (FIG. 6c), which exceeds the structural homology predicted by simple sequence alignments of proteins with unrelated function (Chothia et al. EMBO J. 5(4)823-6, 1986; Lesk et al. J Mol Biol, 136(3):225-70.). Notably the yeast enzyme CDD1, used in pyrimidine salvage, edits ectopically expressed apoB mRNA in yeast. (Dance et al. Nucleic Acids Res 29(8): 1772-80). Hence, it is conceivable that the CDA motif of nucleoside metabolism has been co-opted to function on larger RNA or DNA substrates due to variations at several structural components including the active linker site.

Previously, the threading of the APOBEC-1 primary amino acid sequence onto the backbone atomic coordinates of the known crystal structure of E. coli cytidine deaminase dimer indicated that APOBEC-1 structure was consistent with a head-to-head homodimer with the active CD domain of one monomer in apposition with the CD domain of the other monomer (Navaratnam et al. J Mol Biol, (1998) 275(4):695-714). In this model, one of two active deaminase domains is predicted to interact non-catalytically with a specific U from the RNA substrate while the other active domain interacts with the cytidine to be edited (Navaratnam et al. J Mol Biol, (1998) 275(4):695-714). Importantly, dimerization has been shown to be essential fork editing activity (Lau et al. (1994) PNAS 91:8522-26; Navaratnam et al. (1995) Cell 81:187-95; Oka et al. (1997) JBC 272:1456-60). The model also predicted a leucine-rich region (LRR) in the C-terminus of APOBEC-1 as a functional motif characteristic-of cytidine deaminases that function as dimers. The LRR is essential for APOBEC-1 homodimer formation, apoB mRNA editing, APOBEC-1 interaction with ACF, and APOBEC-1 subcellular distribution (Lau et al. (1994) PNAS 91:8522-26; MacGinnitie et al. (1995) JBC 270:14768-75; Navaratnam et al. (1995) Cell 81:187-95; Oka et al. (1997) JBC 272:1456-60).

2. AID

Other putative members of the CDAR family in humans were identified by genomic sequence analyses and include AID (Muramatsu et al. (1999) JBC 274:18740-76; Muramatsu et al. (2000) Cell 102:553-564); Revy et al. (2000) Cell 102:565-76), APOBEC-2 (Liao et al. (1999) Biochem. Biophys. Res. Commun. 260:398-404) and variants of phorbolins, which are also known as the APOBEC3 family (Anant et al., (1998) Biol Chem. 379:1075-81; Jarmuz et al, (2002) Genomics. 79:285-96; Sheehy et al. (2002) Nature 418:646-50; Madsen et al. (1999) J. Invest. Dermatol. 113:162-69). These candidate CDARs have attracted interest because they share homology with the catalytic domain found in APOBEC-1 and the ADARs and they also have interesting physiological circumstances for their expression. One characteristic of the catalytic domain in CDARs and ADARs is the occurrence and spacing of a histidine and two cysteines (or three cysteines), required for the coordination of a zinc atom, also known as the zinc binding domain or ZBD (Grosjean and Benne ((1998); Mian et al. (1998) J. Comput. Biol. 5:57-72). The ZBD of ADARs is distinguishable from that found in cytidine deaminases because the third cysteine in ADARs is located significantly further in primary sequence from the second conserved cysteine residue (Mian et al. (1998) J. Comput. Biol. 5:57-72; Gerber et al. (2001) TIBS 26:376-84). The ZBD of APOBEC-1 is located in the N-terminal half of the protein and modeling has suggested that a pseudo-(nonfunctional) ZBD domain is repeated in the C-terminus (Mian et al. (1998) J. Comput. Biol. 5:57-72).

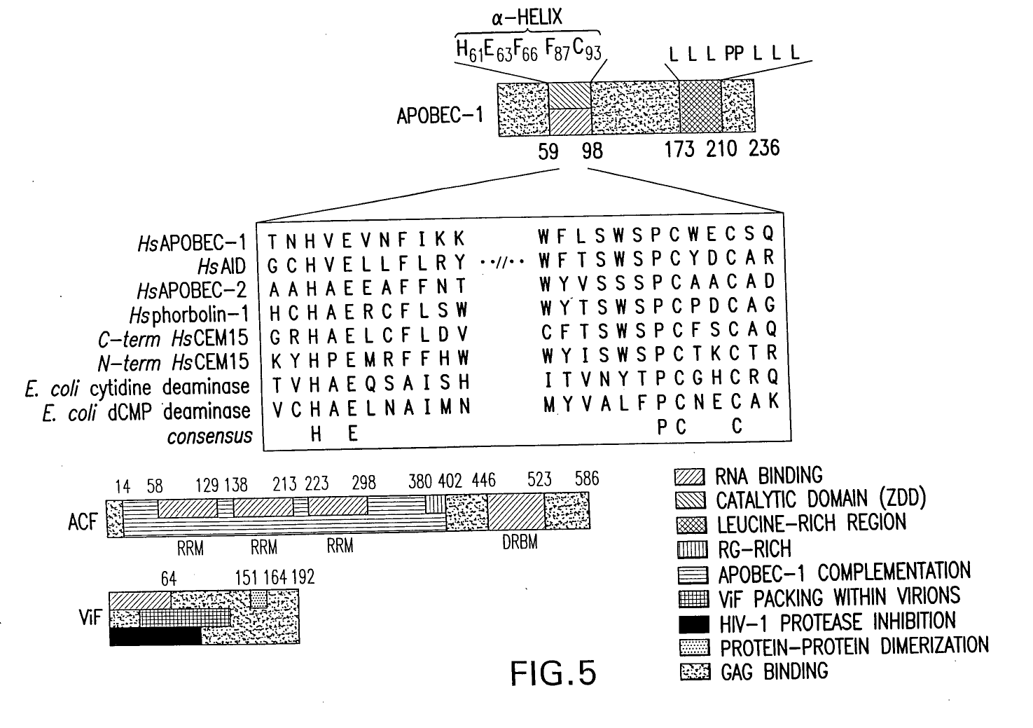

Activation induced deaminase, AID (GenBank accession #BC006296) is encoded on human chromosome 12 (Muto, 2000); (Muramatsu et al. (1999) JBC 274: 18740-76; Muramatsu et al. (2000) Cell 102:553-64; Revy et al. (2000) Cell 102:565-76). AID contains a ZDD (Zinc-dependent deaminase domain) and has 34% amino acid identity to APOBEC-1 (Table 4, FIGS. 5 and 6). Its location on human chromosome 12p 13 suggests it may be related to APOBEC-1 by a gene duplication event (Lau, 1994; Muto, 2000). This chromosomal region has been implicated in the autosomal recessive form of Hyper-IgM syndrome (HIGM2) (Revy, 2000). Most patients with this disorder have homozygous point mutations or deletions in three of the five coding exons, leading to missense or nonsense mutations (Revy, P., 2000) Cell. 102:565-75). Significantly, some patients had missense mutations for key amino acids within AID's ZDD (Revy, 2000; Minegishi, 2000).

AID homologous knockout mice demonstrated that AID expression was the rate limiting step for class switch recombination (CSR) and required for an appropriate level of somatic hypermutation SHM (Muramatsu, 2000). The expression of AID controls antibody diversity through multiple gene rearrangements involving mutation of DNA sequence and recombination. The initial expression of antibodies requires immunoglobulin (Ig) gene rearrangement that is AID-independent (Muramatsu, M., et al., (2000) Cell 102:553-63). This occurs in immature B lymphocytes developing in fetal liver or adult bone marrow and requires DNA double strand breaks at the Ig heavy chain locus whose ends are rejoined by non-homologous end joining. The rearranged immunoglobulin V (variable), D (diversity) and J (oining) gene segments encode a variable region that is expressed initially with the mu (μ) constant region (Cμ) to form a primary antibody repertoire composed of IgM antibodies. In humans and many mammals, AID-dependent gene alterations occur in B lymphocytes that are growing in germinal centers of secondary lymphoid organs following antigen activation. This involves multiple mutations of the variable region through Somatic Hypermutation (SHM) as well as removing the Cμ and replacing it with one of several other constant regions (Ca, Cd, Ce or Cg) through a recombination process known as Class Switch Recombination, CSR. In sheep, rabbits and chickens, pre-immune Ig gene diversification is mediated by an AID-dependent process known as gene conversion (GC) in which stretches of nucleotide sequences from one of several pseudogene V elements are recombined into the VDJ exon to generate diversity (Fugmann, S. D. et al., (2002) Science 295:1244-5; Honjo, T., et al., (2002) Annu Rev Immunol. 20:165-96.). Overexpression of AID in mouse fibroblasts and Ramos B cells induced CSR on an Ig reporter gene and stimulated the rate of SHM respectively (Muramatsu, M. et al. (2000) Cell. 102:553-63; Okazaki, I. M. et al. (2002) Nature. 416:340-45). Given AID's similarity to APOBEC-1, these genomic alterations have been proposed to be due to AID-dependent mRNA editing (Muramatsu, 2000). Editing could promote CSH and SHM through the expression of a novel protein or by reducing the expression/function of an inhibitory protein through alternative exon splicing or codon sense changes.

AID cannot substitute for APOBEC-1 in the editing of apoB mRNA (Muramatsu, 1999) and, although this negative result may have been expected (given that most editing enzymes have substrate specificity (Grosjean and Benne (1998)), it did suggest that AID may have another activity. A competing hypothesis for AID's role in CSR and SHM is that it deaminates deoxycytidine in DNA (Rada, C. et al. (2002) Proc. Natl. Acad. Sci USA. 99:7003-7008; Petersen-Mahrt, S. K., et al., (2002) Nature. 418:99-104). The mutations observed in SHM (and those that arise proximal to the junctions of CSR) are C-T transitions (Yoshikawa, K., et al., (2002) Science, 296:2033-2036). Like APOBEC-1, AID has cytidine and deoxycytidine deaminase activity (Muramatsu, 1999) and its ZDD is homologous to that of E. coli deoxycytidine deaminase (FIG. 5).

AID overexpression in NIH 3T3 fibroblasts resulted in the deamination of deoxycytidine in DNA encoding a green fluorescent protein (GFP) (Yoshikawa, 2002) and also in antibiotic resistance and metabolic genes when AID expression in bacteria was placed under selection for a ‘mutator’ phenotype (Harris, 2002). A variety of mutations were observed on GFP DNA including deletions and duplications; however, a preference for transitions at G/C base pairs clustered within regions predicted to have DNA secondary structure was observed. Similar mutations were observed in the bacteria overexpressing AID and their frequency was markedly enhanced when evaluated in an ung-1 background (lacking functional uracil-DNA glycosylase, an enzyme involved in repairing C to T mutations). (Harris, 2002). These findings together with the observation that the mutation frequency of the GFP gene was 4.5×10⁻⁴/bp per cell generation, which was comparable to the 10⁻³ to 10⁻⁴ frequency observed on Ig genes in B cells, show that AID can act on DNA. The target hotspot for AID is characterized by the motif RGYW (R is A or G, Y is C or T and W is A or T) (Honjo et al. Annu Rev Immunol 20:165-96, 2002; Martin et al. Nat Rev Immunol, 2(8):605-14, 2002).

No mutation hotspot was identified for APOBEC-1 and CEM15 although they have distinct substrate specificities (Harris et al. Mol Cell 10(5):1247-53, 1996). Actively transcribed DNA was identified as the preferred AID substrate (Chaudhuri et al. Nature 422(6933):726-30, 2003), and specifically that dC is deaminated to dU in the strand of DNA that is displaced by transcription of RNA (the non-templating strand); corroborating other studies in which AID selectively deaminated dC in ssDNA or mutated dsDNA reporters within a nine base pair mismatch (the size of a transcription bubble) (Bransteitter et al. Proc Natl Acad Sci USA 100(7):4102-7, 2003; Ramiro et al. Nat Immunol, 2003). AID appears to act processively on DNA, binding initially to RGYW and mutating dC to dU and then modifying multiple dC residues from that point along the same strand of DNA. AID's ability to act on DNA would not negate the possibility that it also acts on RNA. Whether AID is involved in DNA and/or RNA modification, its function clearly results in the diversification of expressed genomic sequences.

3. APOBEC-2

Human APOBEC-2 (Genbank Accession #XM004087) is encoded on chromosome 6 and is expressed uniquely in cardiac and skeletal muscle (Liao et al. Biochem Biophys. Res. Commun. 260:398-404). It shares homology with APOBEC-1's catalytic domain, has a leucine/isoleucine-rich C-terminus and a tandem structural homology of the ZBD in its C-terminus. APOBEC-2 deaminated free nucleotides in vitro but did not have editing activity on apoB mRNA.

4. CEM15/APOBEC-3

Human phorbolin 1, phorbolin 1-related protein, phorbolin-2 and -3 share characteristics with C to U editing enzymes. Several proteins with homology to APOBEC-1 named Phorbolins 1, 2, 3, and Phorbolin-1 related protein were identified in skin from patients suffering from psoriasis and were shown to be induced (in the case of Phorbolins 1 and 2) in skin treated with phorbol 12-myristate-1-acetate (Muramatsu, M. et al. (1999) J Biol Chem. 274:18470-6). The genes for these proteins were subsequently renamed as members of the APOBEC-3 or ARCD family locus (Table 1) (Madsen, P. et al. (1999) J Invest Dermatol. 113:162-9). Bioinformatic studies revealed the presence of two additional APOBEC-1 related proteins in the human genome. One is an expressed gene (XM_{—}092919) located just 2 kb away from APOBEC-3G, and is thus likely to be an eighth member of the family. The other is at position 12q23, and has similarity to APOBEC-3G.

APOBEC-3 variants show homology to cytidine deaminases (FIG. 6d). As anticipated from the SBSA, some of these proteins bind zinc and have RNA binding capacities similar to APOBEC-1 Jarmuz, A., et al., (2002) Genomics, 79:285-96). However, analysis of APOBEC-3A, -3B and -3G revealed them unable to edit apoB mRNA Jarmuz, A., et al., (2002) Genomics, 79:285-96); Muramatsu, M. et al. (1999) J Biol Chem. 274:18470-6). It has been shown that the frequency of deleterious mutations in HIV and impaired infectivity correlated with the expression of CEM15 (APOBEC-3G) (Sheehy et al, 2002; Mariani et al, 2003; Mangeat et al, 2003; Harris et al, 2003; Lecossier et al, 2003. HIV expressing functional Vif (viral infectivity factor) protein was able to overcome the effects of CEM15 due to the ability of Vif to bind and target fit or ubiquitinate and distruct in the proteasome (Mariani et al., Cell 114:21-31, 2003; Stopal et al. Mol. Cell 12:591-601, 2003; Yu et al. Nat Struct Mol. Biol 11:435-42, 2004). In contrast, it is unlikely that APOBEC-3D and 3E functions as an APOBEC-1 like editase because it is missing fundamental sequence elements that are required for mRNA editing by both APOBEC-1 and CDD1 (Anant, S. et al. (2001) Am J Physiol Cell Physiol. 281:C1904-16; Dance et al 2001) and experimental evidence shows it has impaired ability to coordinate Zn⁺ and deaminate cytidine Jarmuz, A., et al., (2002) Genomics, 79:285-96). APOBEC-3E appears to be a pseudogene (Jarmuz, A., et al., (2002) Genomics, 79:285-96), yet the EST database shows that APOBEC-3D and APOBEC-3E are alternatively spliced to form a single CD-PCD-CD-PCD encoding transcript. Additionally, it has been shown that rat APOBEC-1, mouse APOBEC-3, and human APOBEC-3B, are able to inhibit HIV infectivity even in the presence of Vif. Like APOBEC-3G, human APOBEC-3F preferentially restrict vif-deficient virus. The mutation spectra and expression profile of APOBEC-3F indicate that this enzyme, together with APOBEC-3G, accounts for the G to A hypermutation of proviruses described in HIV-infected individuals (Bishop et al., Curr. Bio. 14:1392-1396, 2004). In accordance with this, it has also been shown that APOBEC-3F blocks HIV-1 and is suppressed by both the HIV-1 and HIV-2 Vif proteins (Zheng et al, J Virol 78(11): 6073-6076, 2004; Wiegand et al, EMBO 23:2451-58, 2004). The limited tissue expression, and association with pre-cancerous and cancerous cells (Table 1), and in the case of APOBEC-3G, antagonism of the HIV viral protein Vif shows specific roles for the APOBEC-3 family in growth/cell cycle regulation and antiviral control.

APOBEC-3G (CEM15) has also been shown to interfere with other retroelements, including but not limited to hepatitis B virus (HBV) and murine leukemia virus (MLV). The methods and compositions described herein are useful with any of these viruses (Bishop et al., Curr. Bio. 14:1392-1396, 2004; Machida et al., PNAS 101(12):4262-67, 2004; Turelli et al., Science, 303:1829, 2004).

Table 1 shows APOBEC-1 and related proteins have been described previously (Anant, S., et al., Am J Physiol Cell Physiol. 281:C1904-16; Dance, G. S., et al., (2001) Nucleic Acids Res. 29:1772-80; Jarmuz, A., et al., (2002) Genomics. 79:285-96) and extended through amino acid similarity searches with the (1) hidden Markov modeling software SAM trained with CDD1, APOBEC-1, APOBEC-2, AID and Phorbolin 1, (2) PHI-BLAST, using the target patterns H(V/A)-E-X-X-F-X₁₉-(I/V)-(TNV)-(W/C)-X-X-S-W-(S/T)-P-C-X-X-C (SEQ ID NO: 60) and (H/C)-X-E-X-X-F-X_{(19,30)}-P-C-X_{(2,4)}-C (SEQ ID NO: 61) (FIG. 6a, where X=any amino acid) The gene name and its chromosomal location are indicated and the Accession number of the encoded protein listed. Equivalent/former names are derived from GenBank (Anant, S., et al., (1998) Biol Chem. 379:1075-81; Sheehy, A. M., et al., (2002) Nature 418:646-650). The major tissues of expression are listed. More extensive listings, especially for neoplastic tissues, can be found in the LocusLink pages of Genbank for the individual ARPs which can be accessed from the Unigene Cluster entries. Other human APOBEC-1 Related Proteins (hsARP) (HsARP-6, HsARP-7, HsARP-8, HsARP-10 and HsARP-1 1) only EST data exists as evidence of a final protein product.

TABLE 1

| Gene/ | Protein | Equivalent/Former | | Proposed | |
|---|---|---|---|---|---|
| Chromosomal Location | Accession # | Names/Variants (Accn #) | Expression | CDAR/ARP | Unigene Cluster |
| Yeast | NP_013346 | — | yeast | SeCDAR-1 | |
| CDD1/Chr XII | | | | | |
| [symbol] | | | | | |
| APOBEC-1/12p13.1 | AAD00185 | — | small intestine, liver | HsCDAR-1 | Hs.560 |
| APOBEC-2/6p21 | NP_006780 | CAB44740 | cardiac & skeletal muscle | HsARP-1 | Hs.227457 |
| | | ARCD-1 | | | |
| AID/12p13 | NP_065712 | — | B lymphocytes | HsARP-2 | Hs.149342 |
| APOBEC-3A/22q13.1 | NP_663745 | Phorbolin-1 (P31941) | keratinocytes | HsARP-3 | Hs.348983 |
| APOBEC-3B/22q13.1 | Q9U1117 | Phorbolin-3 | keratinocytes/ | HsARP-4 | Hs.226307 |
| | | Phorbolin-1-related (U61084) | colon | | (specific to U61084 |
| | | Phorbolin-2 (Q9UE74) | | | not APOBEC-3B) |
| | | APOBECIL | | | |
| | | ARCD-3 | | | |
| APOBEC-3C/22q13.1 | CAB45271 | Phorbolin-1 (AF165520) | spleen/testes/heart/thymus | HsARP-5 | Hs.8583 |
| | | ARCD-2/ARCD-4 | prostale/overy/uterus/PBLs | | |
| APOBEC-3D/22q13.1 | BF841711 | — | head & neck cancers | HsARP-6 | |
| | (EST only) | | | | |
| APOBEC-3E/22q13.1 | PSEUDOGENE | ARCD-6 | — | — | |
| APOBEC-3DF3E/22q13.1 | NM_145298 | — | uterus | HsARP-7 | |
| APOBEC-3F/22q13.1 | BG 758984 | ARCD-5 | B lymphocytes | HsARP-8 | |
| | (EST only) | | | | |
| APOBEC-3G/22q13.1 | NP_068594 | Phorbolin-like-protein | spleen/testes/heart/thymus | HsARP-9 | Hs.250619 |
| | | MDS019(AA1124268) | PBLs/colon/stomach/kidney | | |
| | | HsCEM15 | uterus/pancrease/placenta/prostale | | |
| 22q13.1 | XP_092919 | — | — | HsARP-10 | |
| XP_092919 | | | | | |
| 12q23 | XP_115170 | — | — | HsARP-11 | |
| [symbol] | | | | | |
| MmAPOBEC-1/6F2 | NP_112436 | — | small intestine/liver/spleen | MmCDAR-1 | Mm.3333 |
| | | | B lymphocytes/kidney | | |
| MmAPOBEC-2/17 | NP_033824 | — | cardiac & skeletal muscle | MmARP-1 | Mm.27822 |
| | | | brain/skin | | |
| MmAID/6F2 | NP_033775 | | B lymphocytes | MmARP-2 | Mm.32398 |
| CEM15/15 | NP_084531 | XP_122858 | mammory tumour | MmARP-3 | Mm89702 |

Human HIV-1 virus contains a 10-kb single-stranded, positive-sense RNA genome that encodes three major classes of gene products that include: (i) structural proteins such as Gag, Pol and Env; (ii) essential trans-acting proteins (Tat, Rev); and (iii) “auxiliary” proteins that are not required for efficient virus replication in at least some cell culture systems (Vpr, Vif, Vpu, Nef). Among these proteins, Vif is required for efficient virus replication in vivo, as well as in certain host cell types in vitro (Fisher et al. Science 237(4817):888-93, 1987; Strebel et al. Nature 328(6132):728-30, 1987) because of its ability to overcome the action of a cellular antiviral system (Madani et al. J Virol 72(12):10251-5, 1998; Simon et al. Nat Med 4(12):1397-400, 1998).

The in vitro replicative phenotype of vif-deleted molecular clones of HIV-1 is strikingly different in vif-permissive cells (e.g. 293T, SUPTI and CEM-SS T cell lines), as compared to vif-non-permissive cells (e.g. primary T cells, macrophages, or CEM, H9 and HUT78 T cell lines). In the former cells, vif-deleted HIV-1 clones replicate with an efficiency that is essentially identical to that of wild-type virus, whereas in the latter cells, replication of vif-negative HIV-1 mutants is arrested due to a failure to accumulate reverse transcripts and inability to generate infectious proviral integrants in the host cell (Sova et al. J Virol 67(10):6322-6, 1993; von Schwedler et al. J Virol 67(8):4945-55, 1993; Simon et al. J Virol 70(8):5297-305, 1996; Courcoul et al. J Virol 69(4):2068-74, 1995). These defects are due to the expression of the host protein CEM15 (Sheehy, A. M., et al., (2002) Nature. 418:646-650) in non-permissive cells for vif minus viruses. CEM15 antiviral activity is derived from effects on viral RNA or reverse transcripts (Sheehy, A. M., et al., (2002) Nature. 418:646-650). CEM15 deaminates dC to dU as the first strand of DNA is being made by reverse transcriptase or soon after its completion, and this results in dG to dA changes at the corresponding positions during second strand DNA synthesis (Harris et al. Cell 113:803-809, 2003).

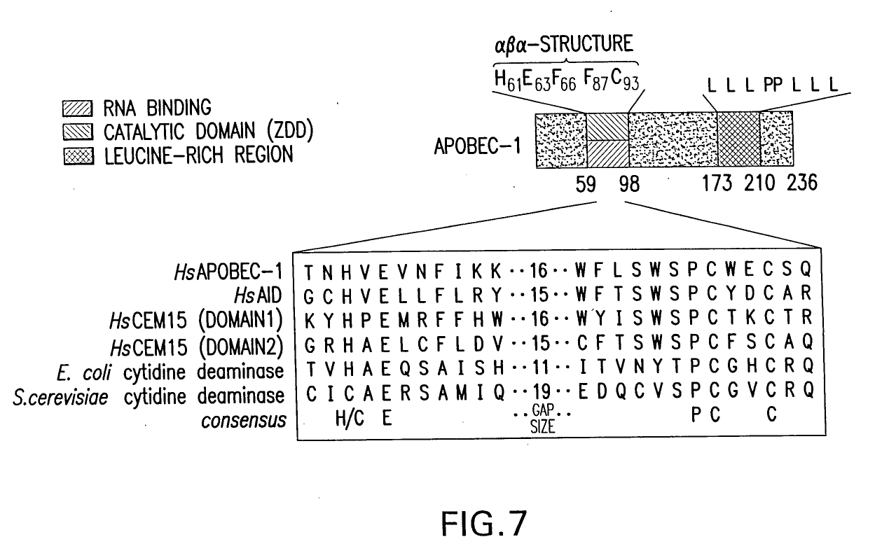

Primary sequence alignments (FIG. 7) and the structural constraints relating CDAs to APOBEC-1 indicate that CEM15 evolved from an APOBEC-1-like precursor by gene duplication (Wedekind et al. Trends Genet 19(4): p. 207-16, 2003). The resulting CEM15 structure exhibits two active sites per polypeptide chain with the topology CD 1-PCD1-connector-CD2-PCD2 (FIG. 8). Knowledge of the structural homology among CDAs and ARPs is sufficient to understand how features of CEM15 contribute to its anti-viral activity. TABLE I

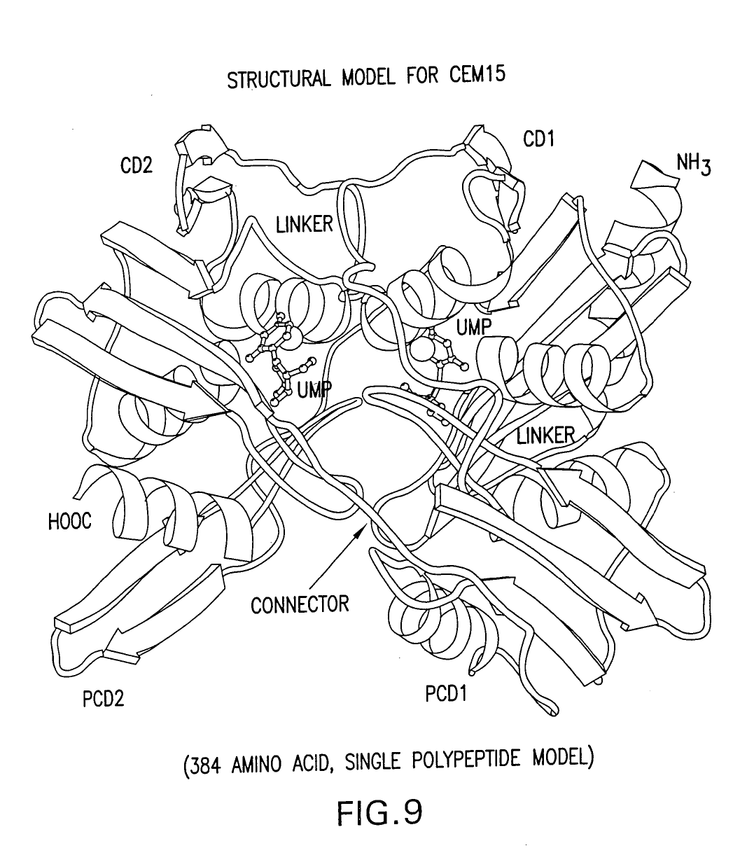

The premise of molecular modeling is that primary sequence analysis alone is insufficient to evaluate effectively the anti-viral activity of CEM15. The use of comparative modeling of CEM15 is based on three known CDA crystal structures (Betts et al. J Mol Biol 235(2):635-56, 1994; Johansson et al. Biochemistry 41(8): p. 2563-70, 2000) and knowledge gained from similar work with APOBEC-1. CEM15 modeling has been accomplished by aligning its amino acid sequence onto a composite three-dimensional template derived by superposition (Winn et al. J Synchrotron Radiat, (2003) 10(Pt 1):23-5; Kabsch et al. Acta. Crystallogr., (1976) A32:922-923; Potterton et al. Acta Crystallogr D Biol Crystallogr, (2002) 58(Pt 11): p. 1955-7) of known crystal structures, representing dimeric and tetrameric quaternary folds of known CDAs. The CEM15 sequence was modeled manually onto three dimensional template using the computer graphics package O (Jones et al. Acta Crystallogr A, (1991) 47 (Pt 2):110-9), thereby preserving the core ZDD fold; gaps and insertions were localized to loops and modeled according to one of the three known structures, or by use of main-chain conformational libraries available in O; similarly amino acid side-chains rotamers were modeled using rotamer libraries (Jones et al. Acta Crystallogr A, (1991) 47 (Pt 2): 110-9). Subsequently, a comparative model was created by use of the program ‘Modeller’ (Sali et al. Proteins, (1995) 23(3):318-26) and subsequently checked by Verify3D (Bowie et al. Science, (1991) 253(5016):164-70; Eisenberg et al. Methods Enzymol, (1997) 277:396-404). The model was energy minimized using simulated annealing and molecular dynamics methods including the CHARM2 energy parameters. No restraints were placed on secondary elements, except those derived from the triple CDA model alignment. The resulting model (FIG. 9) demonstrates that the 384 amino acid sequence of CEM15 can be accommodated by a 222 CDA quaternary fold (analogous to the E. coli CDA or APOBEC-1 with 2×236 amino acids). Albeit CEM15 adopts a CD1-PCD1-CD2-PCD2 tertiary structure with pseudo-222 symmetry (FIG. 9) on a single polypeptide chain. The resulting CEM15 model provides a rational basis for the design of four classes of mutants: (ia) active site zinc (FIG. 6b) ligand changes His65Ala (257), Cys97Ala (288), and Cys100Ala (291), (CD2 residues are noted parenthetically) and (ib) active site proton shuttle Glu57Gln (259). Notably, comparable type (i) mutations in other CDAs abolish activity (Carlow et al. Biochemistry, (1995) 34(13):4220-4; Navaratnam et al. J Mol Biol, (1998) 275(4):695-714; Kuyper et al. J. Crystal Growth, (1996) 168:135-169); (ii) substitution of the active site tinker with a comparably sized linker sequence from E. coli. This change abolishes ACF-dependent mRNA editing activity by APOBEC-1 in HepG2 cells. The linkers in the first and second active sites of CEM15 are conserved amongst ARPs. However, a 3 amino acid insert exists prior to the first linker in CEM15. The CEM15 model predicts mutation of the sequence of either linker would ablate activity whereas point modification of non conserved residues within insert should not; (iii) mutation of surface residues, e.g. F164 (F350) in the PCD(s) is predicted to disrupt auxiliary factor binding (but not mononucleoside deaminase activity), equivalent to the inactivating F156L mutation in APOBEC-1 (Navaratnam et al. J Mol Biol, (1998) 275(4):695-714). None of these mutations is expected to significantly disrupt the CEM15 polypeptide fold, but rather, will help localize regions of the structure necessary for anti-viral activity.

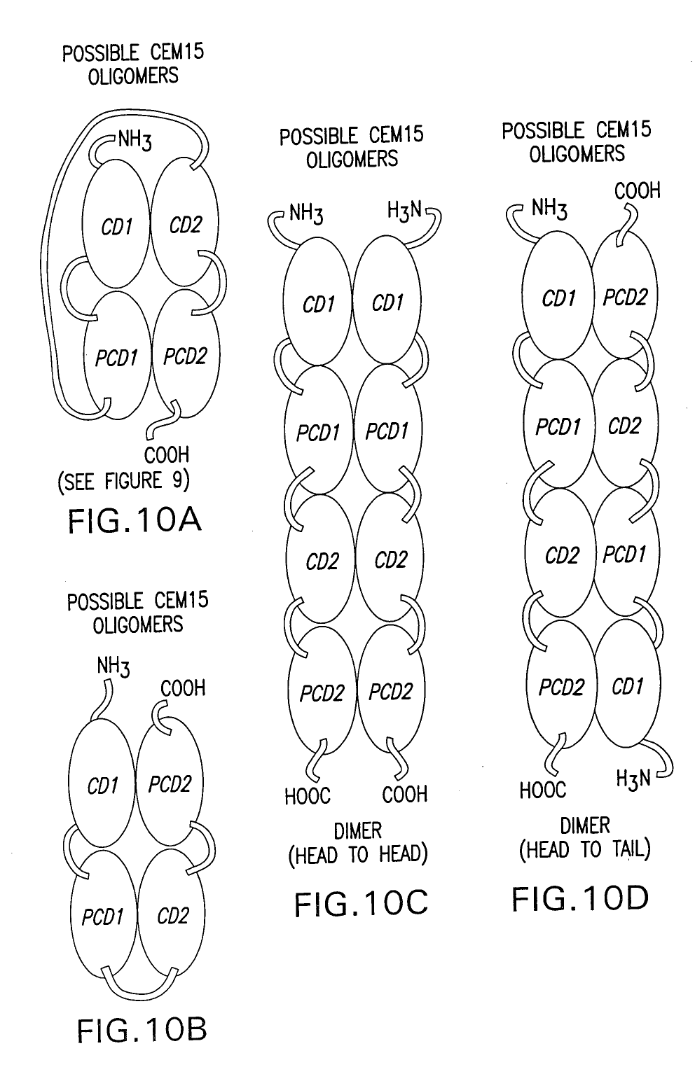

The number of possible CEM15 quaternary structures is limited (FIG. 10); in fact evidence for a dimeric structure has been cited as ‘unpublished’ (Jarmuz et al. Genomics, (2002) 79(3):285-96). Therefore, a fourth class of mutants (truncations) are recognized that can be used to evaluate the requirement of single or dual CD domains for CEM15 activity. Possible dimeric CEM15 structures (FIG. 10) predict mutually exclusive intermolecular contacts. The salient feature of the interaction depicted in FIG. 10c, is that each CD pairs with itself, and similarly for each PCD. In contrast, every domain in FIG. 10d falls in a unique environment (i.e. no CD or PCD pairs with itself). Therefore, to evaluate the need for either single or dual catalytic domain requirements for the anti-viral effect, truncations are expressed. For example, if the dual CD-PCD domain structure were required to ablate viral infectivity, truncation products of the form CD1-PCD1 or CD2-PCD2 would preclude folding of structures depicted in 10a, 10b and 10d, whereas model 10c could fold, leaving open the possibility that either CD1-PCD1 or CD2-PCD2 is sufficient to suppress viral infectivity. Therefore, anti-HIV-1 therapeutics can be designed that disrupt Vif suppression of catalytic activity at either a single CD or both CD1 and CD2 simultaneously. The results of such mutations provide feedback, allowing a more rigorous refinement of the model by use of Modeller (Sali et al. Proteins, (1995) 23(3):318-26). Vif is known to have binding affinity for both viral RNA genomes and a variety of viral and cellular proteins (Simon et al. (1996) J. Virol. 70 (8):5297-5305; Khan et al. (2001) J. Virol. 75(16):7252-7265; Henzler et al. (2001) J. Gen Virol. 82: p. 561-573). Vif also can forms homodimers and homotetramers through its proline rich domain (Yang et al. (2002) J. Biol Chem. 278(8):6596-6602). The infectivity assay in the context of Vif minus pseudotyped viruses and 293 T cells either lacking or expressing CEM15 is found in Example 1. An assay was developed using VSV G-protein pseudotyped lentiviral particles that confirmed the inhibitory effect of CEM15 on the infectivity of vif+ and vif− HIV-1 particles and is amenable to the rapid demarcation of the regions of HIV-1 DNA (or RNA) that is the target for CEM15 catalytic activity.

Also, Vif interacts with CEM15 and induces its poly-ubiquitination and degradation through the proteosome, thereby reducing the abundance of CEML 5 and promoting viral infectivity. It has been discovered that Vif homodimers were required for Vifs interaction with CEM15 (Yang et al. J Biol Chem. 278(8): 6596-602 (2003) and U.S. Pat. No. 6,653,443, herein incorporated by reference in their entirety).

It has been shown that a linker exists between catalytic domains of CEM15. Specifically, human CEM15 contains the amino acid residue “Asp” in the linker between the catalytic domains (Mariani et al. 2003; Bogerd et al. Proc. Natl Acad. Sci. 101:3770-4, 2004; Zhang 2004). A negative to positive charge can be created if Asp or other negatively charged residues are replaced with a positively charged amino acid like lysine, arginine, or histidine, thereby abolishing the binding site of Vif on CEM15. Peptide or small molecule inhibitors that block or compete with binding of Vif to CEM15 can inhibit Vif's ability to block CEM15 activity. The CEM15 binding site of Vif can be similarly targeted, thereby achieving the same goal. Peptides or small molecules that bind the CEM15 binding site of Vif can similarly suppress Vif's effect on CEM15. Thus the Vif antagonists and methods for screening the same can be agents that block the CEM15 linker or the CEM15 binding site.

Agents that prevent Vif mediated polyubiquitination of CEM15 are also desired. Thus, Vif antagonists include agents that block the Vif-mediated polyubiquitination of CEM15. Vif interaction with CEM15 mediates CEM15's interaction with the polyubiquitination machinery, thereby leading to CEM15 conjugation with polyubiquitin (Yu et al Science 2003). This causes CEM15 to be shipped to the proteasome and degraded. By blocking polyubiquitination, CEM15 remains intact and can degrade the retrovirus. Peptides corresponding to the CEM15 sequence that contain the site of ubiquitination can act as mimetics to block ubiquitination of CEM15. Such peptides can be delivered into cells via protein transduction using the aforementioned TAT sequence. Alternatively, Vif must interact with the ubiquitination machinery (Cul5-SCF complex; Yu et al. Science 2003, hereby incorporated by reference in its entirety) and peptide sequences of proteins in this complex can bind to Vif and thereby mimic and block the ability of Vif to target the ubiquitination complex's binding to CEM15. These ubiquitin machinery mimetic peptides can be delivered into cells using the aforementioned protein transduction sequence of TAT.

Disclosed herein are polypeptides comprising 3, 4, 5, 6, 7, 8, 9, 10, 11, 12, 13, 14, 15, 16, 17, 18, 19, 20, or more contiguous amino acid residues of a ubiquitination protein, wherein the polypeptide binds Vif and blocks ubiquitination of CEM15. Also disclosed is a polypeptide comprising 3, 4, 5, 6, 7, 8, 9, 10, 11, 12, 13, 14, 15, 16, 17, 18, 19, 20, or more contiguous amino acid residues ofCEM15 wherien the polypeptide binds a ubiquitination protein and blocks Vif-mediated ubiquitination of CEM15. Also disclosed is a method of blocking the Vif-mediated ubiquitination of CEM15 comprising contacting the CEM15 with the polypeptides disclosed herein.

Also disclosed is a polypeptide comprising 3, 4, 5, 6, 7, 8, 9, 10, 11, 12, 13, 14, 15, 16, 17, 18, 19, 20, or more or more contiguous amino acid residues of a CEM15 binding domain on Vif, wherein the polypeptide blocks CEM15-Vif interaction, as well as a method of blocking CEM15-Vif interaction comprising contacting Vif or CEM15 with the polypeptide disclosed above.

CEM15 contains a Gag binding domain. This binding domain allows for the CEM15 to be packaged into the virus. Vif, however, can block packaging from occurring. Thus, peptide mimetics resemblying the protein sequence of CEM15 that binds to Gag and the the CEM15 protein sequence that binds to Vif can interact with Gag and Vif respectively and thereby block Gag and Vif from binding to CEM15. These peptide memetics enable CEM15 to enter the viral particle during its assembly and prevent the distruction of CEM15, thereby ensuring ample CEM15 to be assembled with virions, respectively.

Disclosed is a polypeptide comprising 3, 4, 5, 6, 7, 8, 9, 10, 11, 12, 13, 14, 15, 16, 17, 18, 19, 20, or more contiguous amino acid residues of a Gag protein, wherein the polypeptide binds CEM15 and promotes CEM15 binding to viral RNA. Also disclosed is a method of promoting CEM15 binding to viral RNA comprising contacting CEM15 with the polypeptide disclosed herein.

Reverse transcription-dependent mutational activity of CEM15 on HIV-1 ssDNA is not the only means by which CEM15 can reduce viral infectivity. Mutations in one or both of the zinc-dependent cytidine deaminase domains did not ablate CEM15's antiviral activity. Moreover, blockage of reverse transcriptase (RT) processivity by CEM15 binding to the viral RNA templates has been indicated as being an additional antiviral mechanism. In support of multiple mechanisms, transient expression of CEM15 reduced the level of pseudotyped HIV-1 particles generated from producer cells that were co-transfected with replication-defective proviral DNA constructs and helper plasmids.

Stably expressed CEM15 significantly reduced the level of pseudotyped HIV-1 particles lacking Vif. The reduced viral particle production is the result of a selective suppression of viral RNA leading to reduction in essential HIV-1 proteins. These effects were not observed when Vif was expressed due to the marked reduction of CEM15. Although CEM15 was required to deplete viral particle production its deaminase function was not necessary. The data indicate an antiviral mechanism in producer cells which is potentially significant late during the viral life cycle that involves directly or indirectly the RNA binding ability of CEM15 and does not require virion incorporation of CEM15 deaminase activity during viral replication. Thus, agents that enhance CEM15 selective binding to viral RNA, leading to viral RNA distruction result in a reduction in viral particle production and a reduced viral burden for the subject. Peptides corresponding to the portion of Gag protein sequence that binds to CEM15 can provide specificity to CEM15 for viral RNA binding by CEM15. TAT transduction of these peptide mimetics activates CEM15 antiviral activity within cells.

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods, specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### B. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

The terms “higher,” “increases,” “elevates,” “enhances,” or “elevation” refer to increases above basal levels, e.g., as compared to a control. The terms “low,” “lower,” “reduces,” “suppresses” or “reduction” refer to decreases below basal levels, e.g., as compared to a control. For example, basal levels are normal in vivo levels prior to, or in the absence of, addition of an agent such as a Vif antagonist or another molecule or ligand.

The term “test compound” is defined as any compound to be tested for its ability to bind to a Vif molecule, a deoxycytidine deaminase molecule, or a cytidine deaminase molecule. Examples of test compounds include, but are not limited to, small molecules such as K+, Ca²⁺, Mg²⁺Fe²⁺ or Fe³+, as well as the anions SO₄²⁻, H₂PO₄⁻ H₃PO₄) and NO³⁻. Also, “test compounds” include drugs, molecules, and compounds that come from combinatorial libraries where thousands of such ligands are screened by drug class.

By “subject” is meant an individual. Preferably, the subject is a mammal such as a primate, and, more preferably, a human. The term “subject” can include domesticated animals, such as cats, dogs, etc., livestock (e.g., cattle, horses, pigs, sheep, goats, etc.), and laboratory animals (e.g., mouse, rabbit, rat, guinea pig, etc.).

The terms “control levels” or “control cells” are defined as the standard by which a change is measured, for example, the controls are not subjected to the experiment, but are instead subjected to a defined set of parameters, or the controls are based on pre-or post-treatment levels.

By “contacting” is meant an instance of exposure of at least one substance to another substance. For example, contacting can include contacting a substance, such as a cell, or cell to a test compound described herein. A cell can be contacted with the test compound, for example, by adding the protein or small molecule to the culture medium (by continuous infusion, by bolus delivery, or by changing the medium to a medium that contains the agent) or by adding the agent to the extracellular fluid in vivo (by local delivery, systemic delivery, intravenous injection, bolus delivery, or continuous infusion). The duration of contact with a cell or group of cells is determined by the time the test compound is present at physiologically effective levels or at presumed physiologically effective levels in the medium or extracellular fluid bathing the cell. In the present invention, for example, a virally infected cell (e.g., a, HIV infected cell) or a cell at risk for viral infection (e.g., before, at about the same time, or shortly after HIV infection of the cell) is contacted with a test compound.

“Treatment” or “treating” means to administer a composition to a subject or a system with an undesired condition or at risk for the condition. The condition can be any pathogenic disease, autoimmune disease, cancer or inflammatory condition. The effect of the administration of the composition to the subject can have the effect of but is not limited to reducing the symptoms of the condition, a reduction in the severity of the condition, or the complete ablation of the condition.

By “effective amount” is meant a therapeutic amount needed to achieve the desired result or results, e.g., editing nucleic acids, interrupting CEM15-vif binding, reducing viral infectivity, inducing class switch recombination, somatic hypermutation, enhancing or blunting physiological functions, altering the qualitative or quantitative nature of the proteins expressed by cell or tissues, and eliminating or reducing disease causing molecules and/or the mRNA or DNA that encodes them, etc.

Herein, “inhibition” or “suppression” means to reduce activity as compared to a control (e.g., activity in the absence of such inhibition). It is understood that inhibition or suppression can mean a slight reduction in activity to the complete ablation of all activity. An “inhibitor” or “suppressor” can be anything that reduces the targeted activity. For example, suppression of CEM15-Vif binding by a disclosed composition can be determined by assaying the amount of CEM15-Vif binding in the presence of the composition to the amount of CEM15-Vif binding in the absence of the composition and by decrease and increase (respectively) in viral infectivity. In this example, if the amount of CEM15-Vif binding is reduced in the presence of the composition as compared to the amount of CEM15-Vif binding in the absence of the composition, the composition can be said to suppress the CEM15-Vif binding.

Many methods disclosed herein refer to “systems.” It is understood that systems can be, for example, cells, columns, or batch processing containers (e.g., culture plates). A system is a set of components, any set of components that allows for the steps of the method to performed. Typically a system will comprise one or more components, such as a protein(s) or reagent(s). One type of system disclosed would be a cell that comprises both Vif and a test compound, for example. Another type of system would be one that comprises a cell and an infective unit (e.g., an HIV unit). A third type of system might be a chromatography column that has CEM15, AID, or other deaminase or putative deaminase, bound to the column.

By “virally infected mammalian cell system” or “virally infected” is meant an in vitro or in vivo system infected by a virus. Such a system can include mammalian cellular components; mammalian cells, tissues, or organs; and whole animal systems. By “HIV infectivity” or “viral infectivity” is meant the capacity of an in vitro ,or in vivo system to become infected by an virus (e.g., an HIV virus).

By “Vif antagonist” is meant any molecule or composition that counteracts, reduces, suppresses, inhibits, blocks, or hinders the activity of a Vif molecule or a fragment thereof. This includes Vif dimerization antagonists, which reduce, suppress, inhibit, block, or hinder the dimerization of Vif. Any time a “Vif antagonist” is mentioned, this includes Vif dimerization antagonists. Also included are agents that block Vif binding to the CEM15, agents that block Vif-mediated polyubiquitination of CEM15, and the like.

By “cytidine deaminase activator” is meant any molecule or composition that enhances or increases the activity of a cytidine deaminase molecule or a fragment thereof. By cytidine deaminase activator is also meant deoxycytidine deaminase activator, ARP activator, or any related molecule.

By “deoxycytidine deaminase activator” is meant any molecule or composition that enhances or increases the activity of a deoxycytidine deaminase molecule or a fragment thereof.

By “ARP activator” is meant any molecule or composition that enhances or increases the activity of an APOBEC-1 Related Protein molecule or a fragment thereof.

A “cytidine deaminase-positive cell” means any cell that expresses one ore more cytidine deaminases or deoxycytidine deaminases. Such express can be naturally occurring or the cell can include an exogenous nucleic acid that encodes one ore more selected deaminases.

### C. SCREENING METHODS

Disclosed herein are methods of screening for Vif antagonists, deoxycytidine deaminase activators, or cytidine deaminase activators. The method of screening for Vif antagonists comprises contacting a Vif molecule with a test compound; detecting binding between the Vif molecule and the test compound or detecting other desired interactions (such as CEM15-Vif binding or binding of Vif with proteins of the polyubiquitin machinery or block Gag interaction with CEM15); and screening the test compound that binds the Vif molecule or display another interaction for suppression of viral infectivity. Suppression of viral infectivity by the test compound indicates the test compound is a Vif antagonist. For the identification of Vif antagonists, it is not necessary to know whether Vif interacts with CEM15 or other viral or cellular proteins nor is it necessary to know the region(s) of Vif that is required to inhibit CEM15 activity.

Also provided is a method of screening for a Vif antagonist, comprising contacting a CEM15 molecule with a test compound; detecting binding between the CEM15 molecule and the test compound or detecting other desired interactions (such as CEM15-Vif binding or binding of Vif with proteins of the polyubiquitin machinery or block Gag interaction with CEM15); and screening the test compound that binds the CEM15 molecule for its ability to block binding of Vif with the CEM15 or to suppress viral activity. An agent that blocks binding of Vif to CEM15 or displays other desired interactions is a Vif antagonist, which can be further tested for its ability to suppress viral infectivity.

As discussed above, “suppression” means to reduce activity as compared to a control (e.g., activity in the absence of such inhibition or suppression). It is understood that inhibition or suppression can mean a slight reduction in activity to the complete ablation of all activity. An “inhibitor” or “suppressor” can be anything that reduces activity. For example, suppression of CEM15-Vif binding by a disclosed composition can be determined by assaying the amount of CEM15-Vif binding in the presence of the composition to the amount of CEM15-Vif binding in the absence of the composition. In this example, if the amount of CEM15-Vif binding is reduced in the presence of the composition as compared to the amount of CEM15-Vif binding in the absence of the composition, the composition can be said to suppress the CEM15-Vif binding.

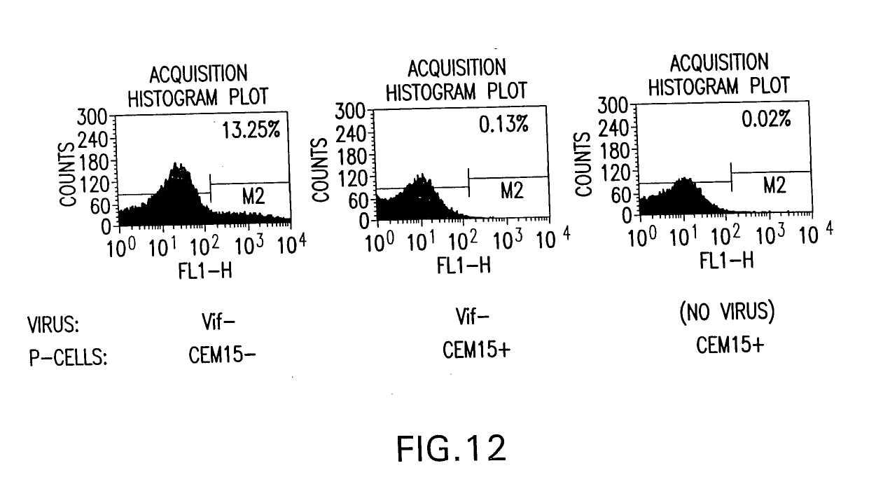

As disclosed in Example 4, an infectivity assay was carried out in the context of Vif minus pseudotyped viruses and 293 T cells either lacking or expressing CEM15. The assay confirmed the inhibitory effect of CEM15 on the infectivity of vif+ and vif− HIV-1 particles. The results (FIG. 12) indicate that the expression of CEM15 in 293T cells resulted in at least a 100-fold decrease in Vif-viral infectivity compared to particles generated in parental 293T cells. The low level of GFP expression from vif−, CEM15+particles is indistinguishable from background fluorescence in control cells.

This assay can be extended to include Vif+ proviral DNA controls and the use of deaminase inactivated CEM15 mutants in stable 293T cell lines. The assay is also amenable to the use of several existing HIV-1 proviral isotyped vectors that are deleted for different regions and different amounts of the HIV-1 genome, as well as to other retroviruses. Deleted genes can be provided in trans by co-transfection of suitable expression plasmids. A comprehensive examination of viral proteins and host tRNA^{LYS3} derived from Vif− virions revealed no significant biochemical or priming defects (Gaddis et al. J. Virol 77(10):5810-5820, 2003.) Dissection of such modifications can be performed in pseudotype viral assays in which key infectivity factors can be rapidly identified and assayed.

The screening assay described herein is useful for detecting Vif antagonists, deoxycytidine deaminase activators, or cytidine deaminase activators. These can block, prevent, or inhibit dimerization of Vif, block the Vif binding site for CEM15 or change the charge of CEM15 or compete with the CEM15/Vif binding sites to block or inhibit binding, block polyubiquitination, enhance CEM15 binding to viral RNA, or block Gag interaction with CEM15.

In one example, each cytidine deaminase activator, deoxycytidine deaminase activator, ARP activator, and Vif antagonist test compound can be tested by treating one or more of the cell types expressing a cytidine deaminase or deoxycytidine deaminase, or ARP, with each test compound and by infecting them with HIV-1 pseudotyped virus (or another retrovirus, or HCV or HBV, for example) containing GFP as described above. Within 48 hours post infection, cell culture supernatants containing viral particles can be added to HeLa cells to test their infectivity, as evidenced by the appearance of green fluorescent cells in FACS analysis as described above. Reduction or elimination of green fluorescent cells relative to that observed in infections from producer cells that were not treated with cytidine deaminase activators or Vif antagonists are scored as a positive identification of cytidine deaminase activators, deoxycytidine deaminase activators, or Vif antagonist test compounds.

Vif antagonists, deoxycytidine deaminase activators, or cytidine deaminase activators enable the normal cellular amounts of CEM15 to mutate HIV-1, HCV, HBV, MLV, or any other retrovirus, to the extent that the virus cannot reproduce itself and therefore cannot elicit a productive infection. Vif antagonists enable CEM15 to mutate viral sequence at the level of first strand DNA synthesis and the resultant dC to dU change is templated during second strand DNA synthesis as dG to dA changes. The frequency of these changes is significantly greater than the mutation rate of reverse transcriptase and consequently the mutations in the retroviral genome affect numerous coding sequences at numerous positions, thereby rendering the virus nonfunctional (incapable of producing infectious virions).

The screening methods disclosed herein can be used with a high throughput screening assay, for example. The high throughput assay system can comprise an immobilized array of test compounds. Alternatively, the Vif molecule or the cytidine deaminase molecule can be immobilized. There are multiple high throughput screening assay techniques that are well known in the art (for example, but not limited to, those described in Abriola et al., J. Biomol. Screen 4:121-127, 1999; Blevitt et al., J. Biomol. Screen 4:87-91, 2000; Hariharan et al., J. Biomol. Screen 4:187-192, 1999; Fox et al., J. Biomol. Screen 4:183-186, 1999; Burbaum and Sigal, Curr. Opin. Chem. Biol. 1:72-78, 1997; Jayasena, Clin. Chem. 45:1628-1650, 1999; and Famulok and Mayer, Curr. Top. Microbiol. Immunol. 243:123-136, 1999).

The Vif molecule, deoxycytidine deaminase activator or cytidine deaminase activator can be linked to a reporter, such as luciferase, GFP, RFP, or FITC, for example. Glow luminescence assays have been readily adopted into high throughput screening facilities because of their intrinsically high sensitivities and long-lived signals. The signals for chemiluminescence, bioluminescence, and colorimetric systems such as luciferase and beta-galactosidase reporter genes or for alkaline phosphatase conjugates are often stable for several hours.

Several commercial luminescence and fluorescence detectors are available that can simultaneously inject liquid into single or multiple wells such as the WALLAC VICTOR2 (single well), MICROBETA RTM JET (six wells), or AURORA VIPR (eight wells). Typically, these instruments require 12 to 96 minutes to read a 96-well plate in flash luminescence or fluorescence mode (1 min/well). An alternative method is to inject the test compoundnif molecule/cytidine deaminase/deoxycytidine deaminase molecule into all sample wells at the same time and measure the luminescence in the whole plate by imaging with a CCD camera, similar to the way that calcium responses are read by calcium-sensitive fluorescent dyes in the FLIPR or FLIPR-384 instruments. Other luminescence or fluorescence imaging systems include LEADSEEKER from AMERSHAM, the WALLAC VIEWLUX™ ultraHTS microplate imager, and the MOLECULAR DEVICES CLIPR imager.

PE BIOSYSTEMS TROPIX produces a CCD-based luminometer, the NORTHSTAR™ HTS Workstation. This instrument is able to rapidly dispense liquid into 96-well or 384-well microtiter plates by an external 8 or 16-head dispenser and then can quickly transfer the plate to a CCD camera that images the whole plate. The total time for dispensing liquid into a plate and transferring it into the reader is about 10 seconds.

The Vif molecule and the reporter can also form a chimera. Purified recombinant Vif (e.g., HA/6His or Vif-CMPK-HA/6His, where CMPK is chicken muscle pyruvate kinase) conjugated with fluorescein isothiocyanate (FITC) or a fusion protein of Vif and GFP (see diagram below) can be used in high throughput screening assays.

| | |
|---|---|
| Vif | (∀) |
| | HA/6-His |
| Vif | HA/6- |
| | His |
| Vif | HA/6- |
| | His |

The Vif molecule can be represented by SEQ ID NO: 7, and the HA domain of the molecule can be represented by SEQ ID NO: 46. The Vif-HA/6-His molecule can be represented by SEQ ID NO: 54 as follows:

MENRWQVMIVWQVDRMRIKTWKSLVKHHMYISKKAKEWVYRHHYESTHPR
ISSEVHIPLGDAKLVITTYWGLHTGEREWHLGQGVSIEWRKKRYNTQVDP
DLADKLIHLHYFDCFSDSAIRHAILGHRVRPKCEYQAGHNKVGSLQYLAL
TALITPKKIKPPLPSVRKLTEDRWNKPQKTKGHRGSHTMNGHGYPYDVPD
YAGHHHHHH

Designates a TEV protease cleavage site (or other appropriate protease cleavage site) where a proteolytic cleavage can be performed on recombinant Vif-CMPK so that Vif may be purified free of CMPK prior to its conjugation to FITC. Vif with or without CMPK may be produced depending on which protein produces the highest yield of soluble protein. A similar strategy can be used for Vif-GST, in which GST is glutathione-S-transferase fused to the Vif N-terminus. Vif can be freed from the GST affinity tag by cleavage with PreScission™ protease, and is then suitable for fluorescein labeling. Regions 6His and HA are not drawn to scale. GFP can also be used in conjunction with the Vif molecule. Vif-GFP would not require a protease cleavage site due to its fluorescence; hence GFP-Vif would not require FITC conjugation. For cytidine deaminase or deoxycytidine deaminase activator or ARP activator HTS screening, Vif has been substituted with CEM15 in all of the constructs listed above.

The Vif-TEV-CMPK-HA/6-His molecule can be represented by SEQ ID NO: 58 as follows:

MENRWQVMIVWQVDRMRIKTWKSLVKHHMYISKKAKEWVYRHHYESTHPR
ISSEVHIPLGDAKLVITTYWGLHTGEREWHLGQGVSIEWRKKRYNTQVDP
DLADKLIHLHYFDCFSDSAIRHAILGHRVRPKCEYQAGHNKVGSLQYLAL
TALITPKKIKPPLPSVRKLTEDRWNKPQKTKGHRGSHTMNGHGENLYFQG
MSKHHDAGTAFIQTQQLHAAMADTFLEHMCRLDIDSEPTIARNTGIICTI
GPASRSVDKLKEMIKSGMNVARLNFSHGTHEYHEGTIKNVREATESFASD
PITYRPVAIALDTKGPEIRTGLIKGSGTAEVELKKGAALKVTLDNAFMEN
CDENVLWVDYKNLIKVIDVGSKIYVDDGLISLLVKEKGKDFVMTEVENGG
MLGSKKGVNLPGAAVDLPAVSEKDIQDLKFGVEQNVDMVFASFIRKAADV
HAVRKVLGEKGKHIKIISKIENHEGVRRFDEIMEASDGIMVARGDLGIEI
PAEKVFLAQKMMIGRCNRAGKPIICATQMLESMIKKPRPTRAEGSDVANA
VLDGADCIMLSGETAKGDYPLEAVRMQHAIAREAEAAMFHRQQFEEILRH
SVHHREPADAMAAGAVEASFKCLAAALIVMTESGRSAHLVSRYRPRAPII
AVTRNDQTARQAHLYRGVFPVLCKQPAHDAWAEDVDLRVNLGMNVGKARG
FFKTGDLVIVLTGWRPGSGYTNTMRVVPVPGYPYDVPDYAIEHHHHHH

The Vif-TEV-EGFP-HA/6-His molecule can be represented by SEQ ID NO: 56 as follows:

MENRWQVMIVWQVDRMRIKTWKSLVKHHMYISKKAKEWVYRHHYESTHPR
ISSEVHIPLGDAKLVITTYWGLHTGEREWHLGQGVSIEWRKKRYNTQVDP
DLADKLIHLHYFDCFSDSAIRHAILGHRVRPKCEYQAGHNKVGSLQYLAL
TALITPKKIKPPLPSVRKLTEDRWNKPQKTKGHRGSHTMNGHGENLYFQG
MVSKGEELFTGVVPILVELDGDVNGHKFSVSGEGEGDATYGKLTLKFICT
TGKLPVPWPTLVTTLTYGVQCFSRYPDHMKQHDFFKSAMPEGYVQERTIF
FKDDGNYKTRAEVKFEGDTLVNRIELKGIDFKEDGNILGHKLEYNYNSHN
VYIMADKQKNGIKVNFKIRHNIEDGSVQLADHYQQNTPIGDGPVLLPDNH
YLSTQSALSKDPNEKRDHMVLLEFVTAAGITLGMDELYKYPYDVPDYAHH
HHHH

In general, compounds that modulate the activity of Vif, deoxycytidine deaminases, ARPs, or cytidine deaminases can be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic-or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds (e.g., but not limited to, antibodies, peptides, and aptamers). Synthetic compound libraries are commercially available, e.g., from Brandon Associates (Merrimack, N.H.) and Aldrich Chemical (Milwaukee, Wis.).

Disclosed is a method of screening for cytidine deaminase activators, comprising: contacting a cytidine deaminase molecule with a test compound; detecting binding between the cytidine deaminase molecule and the test compound; and screening the test compound that binds the cytidine deaminase molecule to identify a selected cytidine deaminase fimction, the presence of the selected function indicating a cytidine deaminase activator.

The cytidine deaminase molecule can be CEM15. Therefore, the cytidine deaminase activator can be a CEM15 activator. The selected CEM15 function can be an increase, decrease, or any modification in the activity of the CEM15 or modifications in CEM15 interaction with other proteins (such as Vif) that modulate CEM15 deaminase activity. For example, the activity of CEM15, such as deoxycytidine to deoxyuridine mutation in the first strand of cDNA, can be increased upon binding of a test compound, thereby decreasing or suppressing viral infectivity. Alternatively, the activity of CEM15 can be decreased, wherein the test compound binds CEM15 and the cytidine to uridine editing of mRNA or deoxycytidine to deoxyuridine mutation of DNA is inhibited or suppressed. A decrease in CEM15 activity can decrease its cancer promoting activity, or reduce cancer phenotype, in vitro or in vivo. An example of a decrease in cancer promoting activity in the presence of compounds that bind CEM15 is found in breast cancer.

The ability of a test compound to suppress viral infectivity can be measured by contacting the test compound with a cytidine deaminase molecule in the presence of Vif and a virus. As disclosed above, the assays disclosed herein are useful for detecting Vif antagonists, deoxycytidine deaminase activators, or cytidine deaminase activators. These can block, prevent, or inhibit dimerization of Vif, block the Vif binding site for CEM15 or change the charge of CEM15 or compete with the CEM15/Vif binding sites to block or inhibit binding, block polyubiquitination, enhance CEM15 binding to viral RNA, or block Gag interaction with CEM15.

The CEM15 function can be, but is not limited to, its cytidine to uridine editing of RNA, or its deoxycytidine to deoxyuridine mutation of DNA, or its suppression of viral activity, or its activity on cancerous or precancerous cells. An “increase in CEM15 activity” is defined as a 10%, 20%, 30%, 40%, 50%, 60%, 70%, 80%, 90%, 100%, 2-fold, 10-fold, 100-fold, or 1000-fold increase in the function of the CEM15. A “decrease in CEM15 activity” is defined as a 10%, 20%, 30%, 40%, 50%, 60%, 70%, 80%, 90%, 100%, 2-fold, 10-fold, 100-fold, or 1000-fold decrease in the function of the CEM15.

The cytidine deaminase molecule can also be APOBEC-1. Therefore, the cytidine deaminase activator is an APOBEC-1 activator. In one example, the activity of APOBEC-1 can be increased such that the levels of apoB48 are increased due to cytidine to uridine editing of apoB mRNA and the levels of apoB100 are consequently decreased as compared to a control level. Increasing APOBEC-1 activity can reduce atherogenic risk by promoting the activity of TAT-APOBEC-1 or the activity of APOBEC-1 expression from a transgene. Alternatively, the activity of APOBEC-1 can be decreased by binding of APOBEC-1 and the test compound, wherein the cytidine to uridine editing of mRNA or deoxycytidine to deoxyuridine mutation of DNA is nhibited or suppressed. An example of the decrease in cancer promoting activity in the presence of compounds that bind CEM15 is found in colon or rectal cancers.

The APOBEC-1 function can be, but is not limited to, its cytidine to uridine editing of RNA, or its deoxycytidine to deoxyuridine mutation of DNA, or the increased levels of apoB48 or decreased levels of apoB100 as compared to a control, or its activity on cancerous or precancerous cells. An “increased levels of apoB48” is defined as a 10%, 20%, 30%, 40%, 50%, 60%, 70%, 80%, 90%, 100%, 2-fold, 10-fold, 100-fold, or 1000-fold increase in the level of apoB48 as compared to a control. A “decreased level of apoB100” is defined as a 10%, 20%, 30%, 40%, 50%, 60%, 70%, 80%, 90%, 100%, 2-fold, 10-fold, 100-fold, or 1000-fold decrease in level of apoB100 as compared to a control.

The cytidine deaminase molecule can also be AID. Therefore, the cytidine deaminase activator is an AID activator. In one example, the activity of AID can be increased such that the levels of cytidine to uridine editing or the levels of deoxycytidine to deoxyuridine mutation are increased and the subsequent and cnsequent class switch recombination and/or somatic hypermutation within the immunoglobulin locus of genes within B lymphocytes is increased. Increasing AID activity can enhance the immune response in individuals that are immunocompromised or have become immunodepressed. Increasing AID activity (for example, the AID activity that promotes class switch recombination) can also enhance the growth and proliferation of B cell lymphomas that express or overexpress AID or mutant forms thereof but fail to undergo class switch recombination or somatic hypermutation. Alternatively, the activity of AID can be decreased such that the levels of cytidine to uridine RNA editing or deoxycytidine to deoxyuridine mutation are decreased (for example, the AID activity that promotes somatic hypermutation), thereby reducing cancer promoting activity or cancer phenotype. An example of the decrease in cancer promoting activity in the presence of compounds that bind AID is found in the treatment of B cell lymphomas that express or overexpress AID, thereby creating inappropriate AID edited mRNAs or AID mutated DNA sequences, or mutant forms thereof. These cells may or may not have undergone class switch recombination or somatic hypermutation.

The AID function can be, but is not limited to, its cytidine to uridine editing of RNA, or its deoxycytidine to deoxyuridine mutation of DNA, or the promotion of antibody diversity produced by lymphocytes as compared to antibody production by control lymphocytes, or its activity on cancerous or precancerous cells. “Promotion of antibody diversity” is defined as a 10%, 20%, 30%, 40%, 50%, 60%, 70%, 80%, 90%, 100%, 2-fold, 10-fold, 100-fold, or 1000-fold increase in diversity of antibodies as compared to control lymphocytes. A “decreased level of AID” is defined as a 10%, 20%, 30%, 40%, 50%, 60%, 70%, 80%, 90%, 100%, 2-fold, 10-fold, 100-fold, or 1000-fold decrease in level of AID as compared to a control.

The cytidine deaminase molecule can also be another ARP listed in Table 1. Therefore, the cytidine deaminase activator is an ARP activator. In one example, the activity of ARP can be increased such that the levels of cytidine to uridine editing or the levels of deoxycytidine to deoxyuridine mutation are increased and the subsequent encoded macromolecule affected by RNA editing or DNA mutation and the physiological process dependent on that native sequence of the affected macromolecule is modulated. RNA editing and DNA mutations induced by ARPs can have health promoting activities when appropriate regulated or disease causing activities when dysregulated. Disclosed herein are molecules that can enhance ARP activity through either direct binding to ARPs or by binding to the macromolecules that interact with ARP as natural regulators of ARP activity.

The ARP function can be, but is not limited to, the cytidine to uridine editing of RNA, or the deoxycytidine to deoxyuridine mutation of DNA, or the promotion of health-promoting or disease-causing pathways.

As disclosed above in reference to the Vif antagonist, the cytidine deaminase can also be linked to a reporter, such as luciferase, GFP, RFP, or FITC, for example. The cytidine deaminase or Vif and the reporter can also form a chimera, as disclosed above. As disclosed above, the cytidine deaminase molecule can be CEM15, AID, APOBEC-1, or any other ARP molecule. The sequences corresponding to CEM15, AID, and APOBEC-1 are SEQ ID NOS: 1, 3, and 5, respectively. The corresponding nucleic acid sequences are SEQ ID NOS: 2, 4, and 6, respectively.

The disclosed compositions (e.g., Vif, cytidine deaminase, or their variants or fragments thereof) can be used as discussed herein as either reagents in micro arrays or as reagents to probe or analyze existing microarrays. The compositions can also be used in any known method of screening assays, related to chip/micro arrays. The compositions can also be used in any known way of using the computer readable embodiments of the disclosed compositions, for example, to study relatedness or to perform molecular modeling analysis related to the disclosed compositions.

The effectiveness of the Vif antagonists or the cytidine deaminase activator can be assessed by detecting deaminase activity. Thus, levels of edited viral RNA and/or mutated (edited) viral DNA, wherein elevated levels of edited viral RNA or mutated (edited) viral DNA indicate enhanced deaminase activity. Additionally, levels of cellular RNA and DNA deaminases comprising by detecting levels of edited cellular RNA and/or mutated (edited) cellular DNA.

Disclosed are methods of identifying an inhibitor of an interaction between the deaminase and the viral infectivity factor, Vif, comprising incubating a library of molecules with the deaminase to form a mixture, and identifying the molecules that disrupt the interaction between the deaminase and the viral infectivity factor. There are many ways of disrupting the interaction between the deaminase and Vif, or the CEM15 interaction with Gag, such as blocking, preventing, or inhibiting dimerization of Vif; blocking the Vif binding site for CEM15 such as changing the charge of CEM15 or competing with the CEM15/Vif binding sites to block or inhibit binding; blocking polyubiquitination; enhancing CEM15 binding to viral RNA, or blocking Gag interaction with CEM15.

An isolating step can comprise incubating the mixture with molecule comprising Vif or a fragment or derivative thereof.

Disclosed are methods of identifying an inhibitor or suppressor of an interaction between a deaminase and a viral infectivity factor (e.g., CEM15 and Vif, respectively) comprising incubating a library of molecules with the viral infectivity factor to form a mixture, and identifying the molecules that disrupt the interaction between the deaminase and the viral infectivity factor. The interaction disrupted can comprise an interaction between the viral infectivity factor and an amino acid of deaminase. An isolation step can comprise incubating the mixture with a molecule comprising a cytidine deaminase or fragment or derivative thereof.

### D. COMPOSITIONS

Disclosed are Vif antagonists identified by the screening methods. Also disclosed are cytidine deaminase activators identified by the screening methods. Also disclosed are deoxycytidine deaminase activators identified by the screening methods. Also disclosed are ARP activators identified by the screening methods. The agents can function by interacting with Vif (e.g., Vif antagonist) or interacting with deoxycytidine deaminase or cytidine deaminase (e.g., cytidine deaminase activator). The Vif antagonist can bind or otherwise interact indirectly with Vif, thereby inhibiting its interaction with CEM15. This can include, but is not limited to, blocking, preventing, or inhibiting dimerization of Vif; blocking the Vif binding site for CEM15; changing the charge of CEM15 or competing with the CEM5/Vif binding sites to block or inhibit binding; blocking polyubiquitination; enhancing CEM15 binding to viral RNA, or blocking Gag interaction with CEM15.

The cytidine deaminase activator or deoxycytidine deaminase activator can bind, or otherwise interact, with a cytidine deaminase or deoxycytidine deaminase, thereby enhancing the normal activity of the cytidine deaminase or deoxycytidine deaminase. For example, a cytidine deaminase activator can interact with CEM15 and enhance the binding of CEM15 to a virus. Conversely, a cytidine deaminase activator can interact with the binding of Vif to a CEM15 molecule, thereby suppressing the activity of Vif, and indirectly enhancing CEM15 binding to HIV.

The Vif antagonists, deoxycytidine deaminase activators, ARP activators, and cytidine deaminase activators of the invention can be modified to enhance suppression of viral activity or to lower biotoxicity. Such modification can further enhance desirable properties, such as: more economical production, greater chemical stability, enhanced pharmacological properties (half-life, absorption, potency, efficacy, etc.), altered specificity (e.g., a broad-spectrum of biological activities), reduced antigenicity, and others.

For example, the Vif antagonist or cytidine deaminase molecule can be modified following Lipinski's Rule of Five. Lipinski's Rule of Five is particularly useful when the goals of compound design are (i) to have less than 5 hydrogen donors, (ii) less than 10 hydrogen bond acceptors, (iii) molecular weight of less than 500 Daltons and (iv) the log of the partition coefficient, P (where P=the concentration of the compound in water divided by the concentration of the compound in 1 octanol) is less than 5. The Lipinski Rule of Five is an example of compound modification, however, the invention is not limited to these parameters.

Disclosed are the components to be used to prepare the disclosed compositions as well as the compositions themselves to be used within the methods disclosed herein. Also disclosed are the compositions identified by the methods disclosed therein.

In some cases the compositions of the invention are chimeric proteins. By “chimeric protein” is meant any single polypeptide unit that comprises two distinct polypeptide domains joined by a peptide bond, optionally by means of an amino acid linker, or a non-peptide bond, wherein the two domains are not naturally occurring within the same polypeptide unit. Typically, such chimeric proteins are made by expression of a cDNA construct but could be made by protein synthesis methods known in the art. These chimeric proteins are useful in screening compounds, as well as with the compounds identified by the methods disclosed herein.

The compositions disclosed herein can also be fragments or derivatives of a naturally occurring deaminase or viral infectivity factor. A “fragrnent” is a polypeptide that is less than the full length of a particular protein or functional domain. By “derivative” or “variant” is meant a polypeptide having a particular sequence that differs at one or more positions from a reference sequence. The fragments or derivatives of a full length protein preferably retain at least one function of the full length protein. For example, a fragment or derivative of a deaminase includes a fragment of a deaminase or a derivative deaminase (e.g., APOBEC-1, AID, CEM15, or an activator of a deaminase) that retains at least one binding or deaminating function of the full length protein. By way of example, the fragment or derivative can include a Zinc-Dependent Cytidine Deaminase domain or can include 20, 30, 40, 50, 60, 70 80, 90% similarity with the full length deaminase. The fragment or derivative can include conservative or non-conservative amino acid substitutions. The fragment or derivative can include a linker sequence joining a catalytic domain (CD) to a pseudo-catalytic domain (PCD) and can have the domain structure CD-PCD-CD-PCD or any repeats thereof. The fragment or derivative can comprise a CD. Other fragments or derivatives are identified by structure-based sequence alignment (SBSA) as shown herein. See FIG. 6b that reveals the consensus structural domain attributes of APOBEC-1 and ARPs (FIG. 6c). The fragment or derivative optionally can form a homodimer or a homotetramer. Also disclosed are chimeric proteins, wherein the deaminase domain is a fragment or derivative of CEM15 having deaminase function.

“Deaminases” include deoxycytidine deaminase, cytidine deaminase, adenosine deaminase, RNA deaminase, DNA deaminase, and other deaminases. Optionally, the deaminase is APOBEC-1 (see international patent application designated PCT/US02/05824, which is incorporated herein by reference in its entirety for APOBEC-1, chimeric proteins related thereto, and uses thereof) (Gen Bank Accession #NP₁₃ 001635), REE (see U.S. Pat. No. 5,747,319, which is incorporated herein by reference in its entirety for REE and uses thereof), or REE-2 (see U.S. Pat. No. 5,804,185, which is incorporated herein by reference in its entirety for REE-2 and uses thereof). Deaminases as described herein can include the following structural features: three or more CDD-1 repeats, two or more functional CDD-1 repeats, one or more zinc binding domains (ZBDs), binding site(s) for mooring sequences, or binding sites for auxiliary RNA binding proteins. Deaminases optionally edit viral RNA, host cell mRNA, viral DNA, host cell DNA or any combination thereof. One deaminase described herein is CEM15. CEM15 is homologous to Phorbolin or APOBEC-3G (see, for example, Accession #NP₁₃ 068594). The names CEM15 and APOBEC-3G can be used interchangeably. CEM15 reduces retroviral infectivity as an RNA or DNA editing enzyme.

By “deaminating function” is meant a deamination of a nucleotide (e.g., cytidine, deoxycytidine, adenosine, or deoxyadenosine). Deaminating function is detected by measuring the amount of deaminated nucleotide, according to the methods taught herein, wherein such levels are above background levels (preferably at least 1.5-2.5 times the background levels of the assay.)

Optionally, the Vif fragment or derivative thereof has at least 20, 30, 40, 50, 60, 70, 80, or 90% amino acid similarity with the Vif molecule of SEQ ID NO: 7. Optionally, the APOBEC-1 fragment or derivative thereof has at least 20, 30, 40, 50, 60, 70, 80, or 90% amino acid similarity with the APOBEC-1 molecule of SEQ ID NO: 5. Optionally, the AID fragment or derivative thereof has at least 20, 30, 40, 50, 60, 70, 80, or 90% amino acid similarity with the AID molecule of SEQ ID NO: 3. Optionally, the CEM15 fragment or derivative has at least 20, 30, 40, 50, 60, 70, 80, or 90% amino acid similarity with the CEM15 molecule of SEQ ID NO: 1.

It is understood that, as discussed herein, the use of the terms “homology” and “identity” are used interchangeably with “similarity” with regard to amino acid or nucleic acid sequences. Homology is further used to refer to similarities in secondary and tertiary structures. In general, it is understood that one way to define any known variants and derivatives or those that might arise, of the disclosed genes and proteins herein, is through defining the variants and derivatives in terms of similarity to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent similarity to the stated sequence or the native sequence. For example, SEQ ID NO: 2 sets forth a particular nucleic acid sequence that encodes a CEM15, and SEQ ID NO: 1 sets forth particular sequences of the proteins encoded by those nucleic acids. Also, SEQ ID NOS: 4, 6, and 8 sets forth particular nucleic acid sequences that encode an AID, an APOBEC-1, and a Vif protein, respectively, and SEQ ID NOS: 3, 5, and 7 sets forth particular sequence of the proteins encoded by those nucleic acids. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent similarity to the stated sequence. Those of skill in the art readily understand how to determine the similarity of two proteins or nucleic acids, such as genes. For example, the similarity can be calculated after aligning the two sequences so that the similarity is at its highest level.

Another way of calculating similarity can be performed by published algorithms. Optimal alignment of sequences for comparison may be conducted by the algorithm of Smith and Waterman Adv. Appl. Math. 2: 482 (1981), by the alignment algorithm of Needleman and Wunsch, J. Mol Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of similarity can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent similarity to another sequence refers to sequences that have the recited homology as calculated by any one or more of the calculation methods described above. For example, a first sequence has 80 percent similarity, as defined herein, to a second sequence if the first sequence is calculated to have 80 percent similarity to the second sequence using the Zuker calculation method even if the first sequence does not have 80 percent similarity to the second sequence as calculated by any of the other calculation methods. As another example, a first sequence has 80 percent similarity, as defined herein, to a second sequence if the first sequence is calculated to have 80 percent similarity to the second sequence using both the Zuker calculation method and the Pearson and Lipman calculation method even if the first sequence does not have 80 percent similarity to the second sequence as calculated by the Smith and Waterman calculation method, the Needleman and Wunsch calculation method, the Jaeger calculation methods, or any of the other calculation methods. As yet another example, a first sequence has 80 percent similarity, as defined herein, to a second sequence if the first sequence is calculated to have 80 percent similarity to the second sequence using each of calculation methods (although, in practice, the different calculation methods will often result in different calculated similarity percentages).

Other structural similarities aside from sequence similarity are also disclosed. For example, homology, as noted by similar secondary and tertiary structure, can be analyzed as taught herein. Homologous proteins may have minimal sequence similarity but have a homologous catalytic domain. Thus, deaminases as used herein may be structurally similar based on the structure of the catalytic domain or other domain but have lower than 70% sequence similarity.

Vif antagonists as well as cytidine deaminase activators, deoxycytidine deaminase activators, and ARP activators can be identified using variants and derivatives of cytidine deaminases, deoxycytidine deaminases, or Vif. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 2 and 3 and are referred to as conservative substitutions.

TABLE 2
Amino Acid Abbreviations

| Amino Acid | Abbreviations |
|---|---|
| Alanine | Ala A |
| Allosoleucine | AIle |
| Arginine | Arg R |
| Asparagines | Asn N |
| Aspartic acid | Asp D |
| Cysteine | Cys C |
| Glutamic acid | Glu E |
| Glutamine | Gln Q |
| Glycine | Gly G |
| Histidine | His H |
| Isolelucine | Ile I |
| Leucine | Leu L |
| Lysine | Lys K |
| Phenylalanine | Phe F |
| Proline | Pro P |
| Pyroglutamic acid | Pglu |
| Serine | Ser S |
| Threonine | Thr T |
| Tyrosine | Tyr Y |
| Tryptophan | Trp W |
| Valine | Val V |

TABLE 3
Amino Acid Substitutions
Original Residue Exemplary Conservative Substitutions

Ala; Ser
Arg; Lys; Gln
Asn; Gln; His
Asp; Glu
Cys; Ser
Gln; Asn, Lys
Glu; Asp
Gly; Pro
His; Asn; Gln
Ile; Leu; Val
Leu; Ile; Val
Lys; Arg; Gln;
Met; Leu; Ile
Phe; Met; Leu; Tyr
Ser; Thr
Thr; Ser
Trp; Tyr
Tyr; Trp; Phe
Val; Ile; Leu

Substantial changes in function or immunological identity are made by selecting substitutions that are less conservative than those in Table 3, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

The compositions disclosed herein can be used as targets in combinatorial chemistry protocols or other screening protocols to isolate molecules that possess desired functional properties related to inhibition of the CEM15-Vif, activation of cytidine deaminase or deoxycytidine deaminase, or antagonism of Vif activity.

Given the information herein, molecules that function like the disclosed molecules can be identified and used as discussed herein. For example, the knowledge that CEM15 interacts with Vif indicates targets for identifying molecules that will affect retroviral infectivity. Disclosed are compositions and methods of making these compositions that bind Vif, such that CEM15 binding to Vif is competitively inhibited or suppressed. Also disclosed are compositions and methods of making these compositions that bind (or interact with) cytidine deaminase molecules, such as CEM15. Preferably, the molecules enhance or suppress a cytidine deaminase or deoxycytidine deaminase function. As discussed herein, this knowledge can be used along with, for example, combinatorial chemistry techniques, identify molecules that function as desired, by for example, inhibiting or suppressing CEM15 and Vif binding, or mimic other cytidine deaminases or deoxycytidine deaminases.

The disclosed compositions, such as cytidine deaminases or deoxycytidine deaminases (e.g., CEM15, APOBEC-1, AID, and other ARPs) or Vif can be used as targets for any combinatorial technique to identify molecules or macromolecular molecules that interact with the disclosed compositions in a desired way or mimic their function. The nucleic acids, peptides, and related molecules disclosed herein can be used as targets for the combinatorial approaches.

It is understood that when using the disclosed compositions in combinatorial techniques or screening methods, molecules, such as macromolecular molecules, will be identified that have particular desired properties such as inhibition, suppression, or stimulation or the target molecule's function. The molecules identified and isolated when using the disclosed compositions, such as, CEM15, AID, APOBEC-1, ARPs, or Vif, are also disclosed. Thus, the products produced using the combinatorial or screening approaches that involve the disclosed compositions, such as, CEM15, AID, APOBEC-1, ARPs, or Vif are also disclosed. Such molecules include Vif antagonists and cytidine deaminase activators.

Combinatorial chemistry includes but is not limited to all methods for isolating small molecules or macromolecules that are capable of binding either a small molecule or another macromolecule like Vif or cytidine deaminase (e.g., CEM15), typically in an iterative process. Proteins, oligonucleotides, and sugars are examples of macromolecules. For example, oligonucleotide molecules with a given ftnction, catalytic or ligand-binding, can be isolated from a complex mixture of random oligonucleotides in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind a small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins that either have de novo activity or a modified activity. For example, phage display libraries have been used to isolate numerous peptides that interact with a specific target (U.S. Pat. No. 6,031,071; 5,824,520; 5,596,079; and 5,565,332 which are herein incorporated by reference in their entirety for their material related to phage display and methods relate to combinatorial chemistry).

A preferred method for isolating proteins that have a given function is described by Roberts and Szostak (Roberts R. W. and Szostak J. W. Proc. Natl. Acad. Sci. USA, 94(23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptdyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997)).

Another preferred method for combinatorial methods designed to isolate peptides is described in Cohen et al. (Cohen B. A., et al., Proc. Natl. Acad. Sci. USA 95(24):14272-7 (1998)). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al. modified this technology so that novel interactions between synthetic or engineered peptide sequences could be identified which bind a molecule of choice. The benefit of this type of technology is that the selection is done in an intracellular environment. The method utilizes a library of peptide molecules that attached to an acidic activation domain. A peptide of choice, for example a portion of Vif is attached to a DNA binding domain of a transcriptional activation protein, such as Gal 4. By performing the Two-hybrid technique on this type of system, molecules that bind the extracellular portion of Vif can be identified.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514).

As used herein combinatorial methods and libraries include traditional screening methods and libraries as well as methods and libraries used in interative processes.

The disclosed compositions (including the Vif antagonists, deoxycytidine deaminase activators, ARP activators, and the cytidine deaminase activators) can be used as targets for any molecular modeling technique to identify either the structure of the disclosed compositions or to identify potential or actual molecules, such as small molecules, which interact in a desired way with the disclosed compositions. The compounds disclosed herein can be used as targets in any molecular modeling program or approach.

It is understood that when using the disclosed compositions in modeling techniques, molecules, such as macromolecular molecules, will be identified that have particular desired properties such as inhibition, suppression, or stimulation or the target molecule's function.

One way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, QSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, CA., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which can alter binding, one can also screen libraries of known compounds, including natural products or synthetic chemicals, and biologically active materials, including proteins, for compounds which alter substrate binding or enzymatic activity.

Also described is a compound that is identified or designed as.a result of any of the disclosed methods can be obtained (or synthesized) and tested for its biological activity, e.g., competitive inhibition or suppression of CEM15-Vif binding or inhibition or suppression of retroviral infectivity.

These and other materials are disclosed herein, and it is understood that when combinations, subsets, interactions, groups, etc. of these materials are disclosed that, while specific reference of each various individual and collective combinations and permutation of these compounds may not be explicitly disclosed, each is specifically contemplated and described herein. For example, if a particular CEM15, Vif, AID, APOBEC, Vif antagonist, deoxycytidine deaminase activator, ARP activator or cytidine deaminase activator is disclosed and discussed and a number of modifications that can be made to a number of molecules are discussed, specifically contemplated is each and every combination and permutation of thereof. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Also contemplated are various molecules with different binding sites on the deoxycytidine deaminase or cytidine deaminase and/or the regulatory proteins thereof that interact with the deoxycytidine deaminase or cytidine deaminase activators, inhibitors, or antagonists, and enhance or inhibit activity thereof.

### E. METHODS OF USING THE COMPOSITIONS

Disclosed are methods of interrupting viral infectivity (e.g., retroviral 10 infectivity like HIV infectivity) comprising contacting an infected cell or a cell prior to infection with a Vif antagonist, under conditions that allow delivery of the antagonist into the cell, wherein the antagonist binds with a viral infectivity factor (Vif) or CEM15 to interrupt viral infectivity. Interruption of viral infectivity may occur at different levels, including, for example, at the level of RNA on the incoming virus, on first or second strand cDNA, after dsDNA integration and/or on transcripts from the viral integrin.

By “interrupting viral infectivity” is meant stopping or reducing the production of infective viral genomes. HIV infectivity, for example, is known to depend on a variety of proteins leading to the synthesis of double stranded DNA from single stranded HIV RNA genome and the integration of HIV DNA into the host cell's chromosomal DNA from where it is expressed to form viral genomes and viral proteins necessary for virion production. A Vif antagonist reduces the ability of virion Vif to inactivate cellular processes, thus allowing CEM15 to effectively mutate HIV or alters its replication and chromosomal integration by affecting the editing of a cellular mRNA encoding a protein that blocks the production of infectious HIV.

The Vif antagonists, deoxycytidine deaminase activators, and cytidine deaminase activators described herein can work in a multitude of ways to interrupt viral infectivity. For example, they can block, prevent, or inhibit dimerization of Vif; block the Vif binding site for CEM15 or change the charge of CEM15 or compete with the CEM15/Vif binding sites to block or inhibit binding; block polyubiquitination; enhance CEM15 binding to viral RNA; or or block Gag interaction with CEM15.

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

Disclosed are methods of treating a subject with a viral infection (e.g., HIV infection) or at risk for an infection comprising administering to the subject an effective amount of the Vif antagonist. As used throughout, administration of an agent described herein can be combined with various others therapies. For example, a subject with HIV may be treated concomitantly with protease inhibitors and other agents.

Also disclosed are methods of treating a subject with a viral infection or at risk of an infection with the compounds as described above. The compound can be in water soluble form, and can be administered by the various routes described throughout. One example of administration is oral administration.

A cytidine deaminase activator is an agent that enhances the efficiency of editing. Additional genetic, pharmacologic, or metabolic agents or conditions also modulate the RNA or DNA editing or mutating function of the deaminase. Some of the conditions that modulate editing activity include: (i) changes in the diet, (ii) hormonal changes (e.g., levels of insulin or thyroid hormone), (iii) osmolarity (e.g., hyper or hypo osmolarity), (iv) ethanol, (v) inhibitors of RNA or protein synthesis and (vi) conditions that promote liver proliferation. Thus, the methods of the invention can comprise administering a cytidine activator to the subject and using other conditions that enhance the efficiency of mRNA editing function.

Also disclosed are methods of treating a condition, wherein the condition is a cancer. The cancer can be selected from the group consisting of lymphomas (Hodgkins and non-Hodgkins), B cell lymphoma, T cell lymphoma, myeloid leukemia, leukemias, mycosis fungoides, carcinomas, carcinomas of solid tissues, squamous cell carcinomas, adenocarcinomas, sarcomas, gliomas, blastomas, neuroblastomas, plasmacytomas, histiocytomas, melanomas, adenomas, hypoxic tumours, myelomas, AIDS-related lymphomas or sarcomas, metastic cancers, bladder cancer, brain cancer, nervous system cancer, squamous cell carcinoma of head and neck, neuroblastoma/glioblastoma, ovarian cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, hematopoietic cancers, testicular cancer, colo-rectal cancers, prostatic cancer, or pancreatic cancer.

Also disclosed are methods wherein the condition to be treated is an infectious disease (e.g., a viral disease). Also disclosed are methods, wherein the viral infection can be selected from the list of viruses consisting of Herpes simplex virus type-1, Herpes simplex virus type-2, Cytomegalovirus, Epstein-Barr virus, Varicella-zoster virus, Human herpesvirus 6, Human herpesvirus 7, Human herpesvirus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency cirus, Human T-cell Leukemia virus type-1, Hantavirus, Rubella virus, Simian Immunodeficiency virus, Human Immunodeficiency virus type-1, Vaccinia virus, SARS virus, and Human Immunodeficiency virus type-2.

Also disclosed are methods of treating a bacterial infection. The bacterial infection can include M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

Also disclosed are methods of treating a parasitic infection. The parasitic infection can include Toxoplasma gondii, Plasmodium falciparum, Plasmodium vivax, Plasmodium malariae, other Plasmodium species., Trypanosoma brucei, Trypanosoma cruzi, Leishmania major, other Leishmania species., Schistosoma mansoni, other Schistosoma species., and Entamoeba histolytica.

Also disclosed are methods of treating a fungal infection. The fuingal infection can include Candida albicans, Cryptococcus neoformans, Histoplama capsulatum, Aspergillus fumigatus, Coccidiodes immitis, Paracoccidiodes brasiliensis, Blastomyces dermitidis, Pneomocystis carnii, Penicillium marneffi, and Alternaria alternata.

Vif antagonists, deoxycytidine deaminase activators, ARP activators, and cytidine deaminase activators are of benefit to individuals who are infected as well as to those who have recently been infected or anticipate an exposure to the virus. As new viruses are produced in individuals who are HIV positive, or positive for another retrovirus, Vif antagonist, deoxycytidine deaminase activator, ARP activator, or cytidine deaminase activator treatment will induce mutations as virus infects new cells. Many of the mutated viruses are destroyed by host cell DNA repair mechanism. Those mutated virus that integrate into chromosomal DNA are not able to produce infectious viral particles. The overall effect is reduced viral shedding into body fluids and consequently a reduction in the probability that new contacts with infected individuals will be infectious. Therefore Vif antagonists, deoxycytidine deaminase activators, ARP activators, and cytidine deaminase activators reduce the production of infectious viruses in affected individuals thereby controlling the disease at an early stage and reducing the probability of transmission. For individuals who have been recently exposed or anticipate an exposure (rape victims, a child born to an HIV positive mother, healthcare workers, emergency personnel, disaster management teams, terrorist response teams and paramedics,) Vif antagonists, deoxycytidine deaminase activators, ARP activators, or cytidine deaminase activators can prevent a productive infection from taking place by allowing CEM15 to destroy retroviral genomes before they can be integrated, or rendering those that do integrate nonproductive during their replication.

With all of the methods described herein, the virus can be a retrovirus (e.g., HIV). The virus can be an RNA virus. The RNA virus can be selected from the list of viruses consisting of Vesicular stomatitis virus, Hepatitis A virus, Hepatitis C virus, Rhinovirus, Coronavirus, Influenza virus A, Influenza virus B, Measles virus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Hantavirus, and Rubella virus.

The ability to suppress viral infectivity can be measured by contacting the test compound with one or more cytidine deaminase-positive cells, in the presence of Vif and a virus. Cytidine deaminase positive cells are cells that express a cytidine deaminase molecule or fragment thereof, such as CEM15, APOBEC-1, AID, or ARPs.

Thus, the disclosed compositions can also be used diagnostic tools related to diseases that are susceptible to RNA or DNA editing, such as HIV, HCV, HBV, or MLV.

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, although topical intranasal administration or administration by inhalant is typically preferred. As used herein, “topical intranasal administration” means delivery of the compositions into the nose and nasal passages through one or both of the nares and can comprise delivery by a spraying mechanism or droplet mechanism, or through aerosolization of the nucleic acid or vector. The latter may be effective when a large number of animals is to be treated simultaneously. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. In general, receptors are involved in pathways of endocytosis, either constitutive or ligand induced. These receptors cluster in clathrin-coated pits, enter the cell via clathrin-coated vesicles, pass through an acidified endosome in which-the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

Delivery of the Vif antagonist, deoxycytidine deaminase activator, ARP activator, or cytidine deaminase activator compositions can be used therapeutically in combination with a pharmaceutically acceptable carrier. Pharmaceutical carriers are known to those skilled in the art. These most typically would be standard carriers for administration of drugs to humans, including solutions such as sterile water, saline, and buffered solutions at physiological pH. The-compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including opthamalically, vaginally, rectally, intranasally), orally, by inhalation, or parenterally, for example by intravenous drip, subcutaneous, intraperitoneal or intramuscular injection. The disclosed compounds can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid-or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

The dosage ranges for the administration of the compositions are those large enough to produce the desired effect in which the symptoms disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any contraindications. Dosage can vary, and can be administered in one or more dose administrations daily, for one or several days.

Vif antagonists, deoxycytidine deaminase activators, ARP activators, or cytidine deaminase activators that do not have a specific pharmaceutical function, but which may be used for tracking changes within cellular chromosomes or for the delivery of diagnostic tools for example can be delivered in ways similar to those described for the pharmaceutical products.

As described previously, molecules such as Vif antagonists, deoxycytidine deaminase activators, ARP activators, and cytidine deaminase activators can be administered together with other forms of therapy. For example, the molecules can be administered with antibodies, antibiotics, or TAT peptides. TAT-fusion peptides are especially useful with the methods described herein, as they are rapidly internalized by lipid raft-dependent macropinocytosis and then able to escape. dTAT-HA2 is also useful with the methods disclosed herein, and is transducible, pH-sensitive, and ftisogenic (Wadia et al., Nature Medicine, 10(3):310-315, 2004).

### F. METHODS OF MAKING THE COMPOSITIONS

The compositions disclosed herein and the compositions necessary to perform the disclosed methods can be made using any method known to those of skill in the art for that particular reagent or compound unless otherwise specifically noted.

Also disclosed are methods of making a Vif antagonist, comprising identifying a Vif antagonist by the screening methods disclosed herein; and modifying the Vif antagonist to enhance suppression of viral infectivity. Methods of modifying the Vif antagonist are disclosed herein. The Vif antagonist can be modified by a number of means, as disclosed above, such as using Lipinski's Rule of Five. Such modifications can include amino acid modifications, thereby producing variants and derivatives that enhance suppression of viral activity. Also disclosed are Vif antagonists and cytidine deaminase activators made by the methods described herein.

Disclosed are methods of making a cytidine deaminase activator comprising identifying the cytidine deaminase activator; and modifying the cytidine deaminase activator to enhance the selected deaminase function of the modified cytidine deaminase activator as compared to the function of the unmodified cytidine deaminase activator. Methods of modifying the cytidine deaminase activator are disclosed herein, such as using Lipinski's Rule of Five. The cytidine deaminase activator can be modified by a number of means, as disclosed above. Such modifications can include amino acid modifications, thereby producing variants and derivatives that enhance suppression of viral activity. The same method can be used to make deoxycytidine deaminase activators and ARP activators.

“Suppression of viral activity” is defined as a 10%, 20%, 30%, 40%, 50%, 60%, 70%, 80%, 90%, 100%, 2-fold, 10-fold, 100-fold, or 1000-fold suppression of viral activity. Viral activity includes, but is not limited to, viral reproduction, viral shedding, or viral infectivity.

Also disclosed are methods of making a Vif antagonist, comprising identifying the Vif antagonist by the screening methods disclosed herein; and modifying the Vif antagonist to lower biotoxicity of the test compound.

Also disclosed is a method of making a cytidine deaminase activator comprising identifying the cytidine deaminase activator; and modifying the cytidine deaminase activator to lower biotoxicity of the modified cytidine deaminase activator as compared to the biotoxicity of the unmodified cytidine deaminase activator. The same method can be used to make deoxycytidine deaminase activators and ARP activators.

“Lower biotoxicity” is defined as a 10%, 20%, 30%, 40%, 50%, 60%, 70%, 80%, 90%, 100%, 2-fold, 10-fold, 100-fold, or 1000-fold lowering of the biotoxicity of the test compound. Biotoxicity is defined as the toxicity of the compound to a cell or to a system, in vitro or in vivo.

Disclosed are methods of treating a subject comprising administering to the subject an inhibitor of viral infectivity (e.g., HIV infectivity), wherein the inhibitor reduces the interaction between a deaminase (e.g., CEM15) and a viral infectivity factor (Vif), and wherein the subject is in need of such treatment.

Disclosed are methods of manufacturing a composition for inhibiting the interaction between a deaminase (e.g., CEM15) and a viral infectivity factor (Vif) comprising synthesizing the Vif antagonists as disclosed herein. Also disclosed are methods of manufacturing a composition for enhancing the activity of a deaminase such as CEM15, APOBEC-1, AID, or other ARPs. Also disclosed are methods that include mixing a pharmaceutical carrier with the Vif antagonists, deoxycytidine deaminase activator, ARP activator, or the cytidine deaminase.

Disclosed are methods of making a composition capable of inhibiting infectivity (e.g., HIV infectivity) comprising admixing a compound with a pharmaceutically acceptable carrier, wherein the compound is identified by the methods described herein.

G. CHIPS AND MICROARRAYS

Disclosed are chips comprising nucleic acids that encode Vif, cytidine deaminases, deoxycytidine deaminases, ARPs, or fragments or variants thereof or where at least one address is such a nucleic acid. Also disclosed are chips where at least one address is an amino acid sequence for Vif, deoxycytidine deaminases, ARPs, cytidine deaminases, or fragments or variants thereof.

### H. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. U.S. Provisional Application No. 60/401,293 and PCT/US02/05824, are incorporated herein by reference in their entireties for the examples, methods, and compounds therein.

### 1. Example 1

### a) Methods for Obtaining the CEM15 cDNA and for Cloning it Into Two Different Systems

Human CEM15 (NP-068594; also known as MDS019, AAH24268) was amplified from total cellular RNA of the NALM-6 cell line (human B cell precursor leukemia) by RT-PCR.

Oligo-dT primed first-strand cDNA was amplified using Expand HiFi Taq DNA polymerase (Roche) with the following primers; ‘5′A’ CACTTTAGGGAGGGCTGTCC (SEQ ID NO: 10) and ‘3′A’ CTGTGATCAGCTGGAGATGG (SEQ ID NO: 11). The 1366 bp product was reamplified with CEM15 specific PCR primers that included NcoI and XhoI restriction sites on the 5′ and 3′ primer respectively; ‘5′B’ CTCCCATGGCAAAGCCTCACTTCAGAAACACAG (SEQ ID NO: 12) and ‘3′B’ CTCCTCGAGGTTTTCCTGATTCTGGAGAATGGCCC (SEQ ID NO: 13). The 1154 bp PCR product was digested with EcoRI to remove potentially co-amplified highly homologous APOBEC3B/Phorbolin 3 (Q9UH17) sequences and the NcoI/XhoI digested product subcloned into a modified pET28a (Novagen) plasmid such that a CEM15-thrombin-HA-6His fusion protein could be expressed. The full-length human CEM15 cDNA was subcloned by PCR into a mammalian expression vector (pcDNA3) such that it is expressed with an amino terminal haemagglutinin (HA) epitope. It was also subcloned into pET28a (Novagen) to express a 6His-thrombin-CEM15 fusion protein.

The expression of the former clone in mammalian HepG2 cells (Human liver hepatoma line) demonstrate expression of full length protein (PAGE gel cell extracts were transferred to nitrocellulose and the presence of CEM15 was determined by reaction with anti-HA tag antibodies). This latter fusion was expressed to high levels in E. coli as a soluble protein and purified by nickel affinity chromatography (the expression and yield of CEM15 was determined by Coomassie blue stained PAGE gel and was approximately 700 μg per 50 mls of original E. coli culture).

### 2. Example 2

### a) APOBEC-1 Model

The construction of the APOBEC-1 model is based upon the hypothesis that enzymes with a common catalytic function (i.e. hydrolytic deamination of a nucleoside base) exhibit a common three-dimensional fold despite a low overall amino acid sequence identity (even at levels <30%). This level of homology is often cited as the lower limit upon which one can reliably model the fold of a given polypeptide sequence (Burley, S. K. (2000) Nature Struct. Biol. 7:932-934.). However, the structures of molecules with similar biological functions are known to be highly conserved even at low levels of primary structure homology (Chothia et al. Embo J. 5(4)823-6, 1986; Lesk et al. J Mol Biol, 136(3):225-70.) At present experimentally derived three-dimensional structures are available for three cytidine deaminases (CDAs) whose role in pyrimidine metabolism has been firmly established. These enzymes encompass the dimeric CDA from E. coli (Betts L. et al., C W (1994) J Mol Biol. 235:635-56), the tetrameric CDA from B. subtilis (Johansson E. et al. (2002). Biochem. 41:2563-70) and the tetrameric CDA Cdd1 from S. cerevisiae [Xie et al., & Wedekind, manuscript in preparation]. The Cartesian coordinates for the former two models are available in the public Protein Data Bank (www.rcsb.org/pdb) as entries 1AF2 and 1JTK. Among the known CDA structures however, only Cdd1 exhibits RNA editing activity (Dance, G. S. C. et al. (2001) Nuc. Acids Res. 29:1772-1780.) and therefore its coordinates have been critical in the assembly of a composite 3-D model for APOBEC-1 because it provides direct evidence that the fundamental CDA polypeptide fold is necessary and sufficient for RNA editing and can function as a dC to dU DNA mutator as evidenced by the activity of APOBEC-1 and CEM15. Furthermore, the Cdd1 crystal structure is a critical component in the development of a working model for RNA editing by APOBEC-1 and provides a tool to understand and manipulate its related proteins (ARPs) including AID, and CEM15.

### b) Methods for the Construction of a Structure-Based Sequence Alignment (SBSA) Leading to a New APOBEC-1 Three-Dimensional Model

(1) Expression and Purification Cdd1 was amplified by PCR from Baker's yeast. The product was cloned into a pET-28a vector (Novagen) containing N-terninal 6×His using NdeI and EcoRI restriction sites; constructs were verified by DNA sequencing. BL21 CodonPlus (Stratagene) cells transformed with vector were grown at 37° C. to an OD₆₀₀ of 0.7 and induced with 1 mM. IPTG at 30° C. for 3 hours. Bacterial pellets were resuspended in lysis buffer (50 mM. Tris-Cl pH 8.0, 10 mM. β-mercaptoethanol, 1 mg/ml lysozyme, 1 mM. PMSF, 2 mM. benzamidine and 5 μg/ml each of aprotinin, leupeptin and pepstatin A), lysed, and nuclease digested (0.5% Triton X-100, 2 mM. ATP, 10 mM. MgSO₄, 33 μg/ml each of DNaseI and RNaseI) at 4° C. The 6×His tagged protein was purified in batch with NiNTA agarose (Qiagen) utilizing the following wash, elution, and dialysis scheme: wash 1, 10 mM Tris-Cl pH 8.0, 100 mM. KCl, 20 mM. imidazole, 10% glycerol; wash 2, same as wash 1 including 1M. KCl; wash 3, repeat wash 1; elution, 10 mM. Tris-Cl pH 8.0, 0.5M. KCl, 0.4 M. imidazole, 10% glycerol; dialysis against 2×2 liters 10 mM. Tris-Cl pH 8.0, 120 mM. NaCl, 1 mM. DTT). Removal of the 6×His tag was achieved by digestion for 16 hours at 20° C. with 10 U biotinylated thrombin (Pierce). Protein was dialyzed against 20 mM. HEPPS pH 8.0, 0.25 M. KCl, 5% glycerol, and 4 mM. DTT and concentrated to 6 mg/ml as estimated by Bradford assays (BioRad) using an Ultrafree-4 spin cartridge (Millipore). Protein was utilized immediately for crystallization.

### (2) Crystallization

Crystals were grown at 20° C. from well solutions of 16.5% (w/v) PEG monomethylether (MME) 5K, 450 mM. NH₄Cl, 100 mM. Na-succinate pH 5.5, 10 mM DTT and 1 mM. NaN₃ by use of the hanging drop vapor diffusion method. Four μl of well solution was added to an equal volume of protein. Crystals appeared in six days and reached a maximum size of 50×90×450 mm³ after 3-4 weeks. Single crystals were harvested with a nylon loop (Hampton Research), and cryo-protected through four serial transfers in 100 μl volumes of solutions containing 19% (w/v) PEG monomethylether 5000, 500 mM. NH₄Cl, 100 mM. Na-succinate pH 5.5, 1 mM. DTT and either 5, 10, 15 or 17.5% (v/v) PEG 550MME. Crystals were flash cooled by plunging into liquid nitrogen, and stored for X-ray data collection. In order to bind UMP, crystals were serially transferred in the presence of 10 mM. UMP from pH 5.5 to 7.5 in 0.5 pH unit increments. Buffers of the appropriate pKa were chosen for each step. Crystals were subsequently cryo-adapted at elevated pH and flash frozen as described.

### (3) Structure Determination

Crystals of scCdd1 belong to space group C222₁ with unit cell dimensions a=78.51 Å, b=86.32 Å and c=156.14 Å. There is one 16.5 kDa tetramer (4×145 amino acids) per asu. The structure was solved by use of MAD phasing at the Zn(II) K-absorption edge with the peak energy at 1.2828 Å, inflection=1.28310 Å and remote energy 1.25740 Å. The positions of four zinc atoms were located by use of the program SOLVE v2.0, and phases were density modified by use of the program RESOLVE with 4-fold NCS averaging. The NCS averaged phases improved electron density maps significantly and allowed manual skeletonization by use of O. Additional NCS averaging with DM. improved maps quality and allowed modeling of amino acids 4 to 136 in all four subunits. Upon addition of UMP, the C-terminal 6 amino acids are observed. The present structure has been refined by use of CNS using all data from 30 to 2.0 Å resolution with a crystallographic R_{factor} of 23.2% (R_{free}=26.2%). The model exhibits reasonable bond and angle deviations from ideal values (0.009 Å and 1.52o, respectively) as evaluated by PROCHECK. More than 89% of residues are in the allowed region of the Ramachandran Plot.

### (4) Homology Modeling

The design of homology models for the ARP enzymes was based upon the observation that the enzyme Cdd1 from Saccharomyces cerevisiae is capable of acting on monomeric nucleoside substrates of pyrimidine metabolism, as well as larger RNA substrates such as reporter apoB mRNA expressed ectopically in yeast (Dance et al, 2001 Nucleic Acid Res. 29, 1772-1780). These results along with our X-ray crystallographic structure determination of yeast Cdd1 demonstrated that the fundamental CDA fold, typical of pyrimidine metabolism enzymes, are sufficient for catalyzing C to U editing of RNA or dC to dU mutations on DNA. As such, the three known crystal structure of cytidine deaminases were utilized to prepare a template for homology modeling of APOBEC-1, CEM15 and AID. The initial amino acid sequence alignment among enzymes of known structure with those of the unknown ARPs was prepared by use of the program ClustalX v1.8 (Thompson et al., 1997 Nucleic Acid Res. 24, 4876-4882). Sequences aligned included: #P19079 (B. subtilis), #NP₁₃ 013346 (S. cerevisiae), #1065122 (E. coli), #4097988 (APOBEC-1 from H. sapiens), NP₁₃ 065712(AID from H. sapiens) and #NP₁₃ 068594 (APOBEC-3G from H. sapiens), which were retrieved from the NCBI (www.ncbi.nlm.nih.gov/Pubmed). Subsequently, manual adjustments were made to the alignments of the ARP primary sequences according to sequence constraints derived from the triple three-dimensional structural superposition of the known cytidine deaminase coordinates of yeast (i.e. scCDD1), E. coli (PDB accession number 1AF2) and B. subtilis PDB (PDB accession number 1JTK) described by Betts et al. (1994,J. Mol. Biol 235, 635-56) and Johansson et al. (2002 Biochemistry 41, 2563-70) as implemented in the program LSQKAB (Kabsch 1976 Acta Crystallogr. A 32, 922-923). When optimized to account for the conserved three-dimensional fold, the alignments between the enzymes of pyrimidine metabolism and the ARPs revealed sequence identity ranging from ˜7% to 26% in the respective catalytic and non-catalytic domains. Despite the modest sequence identity at the amino acid level, the actual three-dimensional structural homology of proteins with a common function often far exceeds the relatedness values predicted by simple amino acid sequence alignments (Chothia & Lesk, 1986 EMBO J. 5, 823-826). In order to rigorously model the respective ARP structures with the highest degree of empirically derived structural restraints, the method of comparative modeling was employed using “satisfaction of spatial restraints” as implemented in the program Modeller (Sali & Blundell 1993, J. Mol. Biol. 234, 779-815). Following model calculation, realistic model geometry is achieved through real-space optimization using enforced stereochemical refinement derived from application of the CHARM22 force field parameters (MacKerell et al., 1998 J. Phys. Chem. B. 102 3586-16). In all models, the Zn²⁺ ion was constrained in Modeller to be within 2.25 Å distance of each the respective putative metal ligands: 2× cyteine-Sγ and 1× histidine-Nδ1. This constraint resulted in a satisfactory and realistic tetrahedral geom etry consistent with the known CDA structures, as well as the chemical requirements for base hydrolytic deamination. In order to model the location of DNA or RNA substrate binding, the edited nucleotide was modeled according to constraints derived from the known locations of CDA inhibitors in the template X-ray crystal structures: 1JKT (tetrahydrouridine ) and 1AF2 (3,4 dihydrouridine). Due to the known substrates of AID and APOBEC-1, DNA and RNA sequences were modeled as single-stranded. Additionally, the restraint that nucleotide bases flanking the edited/mutated sites maintain modest base stacking was imposed by adding additional distance restraints in the model calculation. Each monomer of a respective ARP model was also restrained to be symmetric. This method of modeling far exceeds previous standards employed to model APOBEC-1 (Navaratnam, N. et al. (1998) JMB 275:695-714.). The surprising result of modeling is the existence of an extensive flexible linker that extends from residues 136 to 143 of human APOBEC-1 and residues 131-138 of human AID.

### (5) Mutagenesis and Construction of Chimeric Cdd1 Enzymes

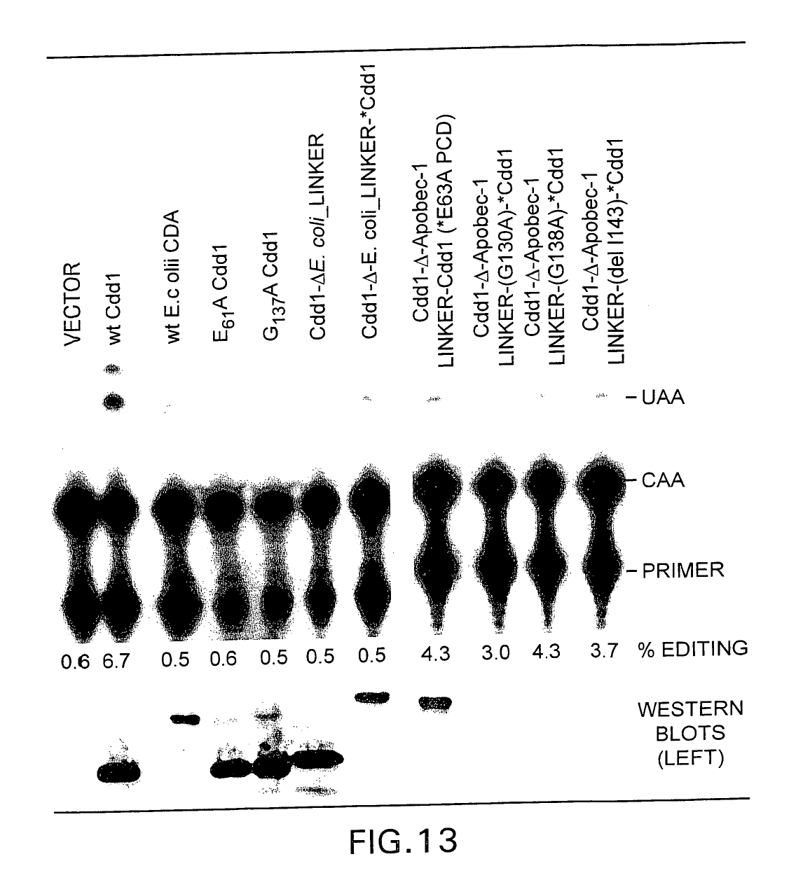

In order to corroborate the comparative model of APOBEC-1, the Cdd1 was employed as a model compound to examine: (i) the feasibility of the predicted APOBEC-1 fold, and (ii) the role of key functional elements predicted to be in the active site linker or other active site locations necessary for catalysis. (Mutations can be divided into two classes: those that stabilize/destabilize the structure through insertions or changes of large stretches of amino acids; and those that effect function by modest changes to amino acids). A series of mutants were constructed in a manner analogous to the following method. In order to assess the importance of the predicted C-terminal “tail” of Cdd1 upon the ability to edit RNA, a 19 amino acid linker from E. coli was added after residue 142. Specifically, Cdd1 was PCR amplified using a 5′ Cdd1 -specific primer and a 3′ primer encoding the 19 amino acid E. coli “linker” extension and subcloned into the NdeI and EcoRI sites of pET28a (Novagen). In order to assess the importance of linker flexibility Gly137 was converted to Ala using the QuikChange mutagenesis system (Stratagene) according to the manufacturer's protocols; other point mutations were constructed similarly. To assesses whether or not the CDA from E. coli (PDB #1AF2) was competent to edit under conditions similar to APOBEC-1 and Cdd1 in yeast (Dance et al., 2001 Nucleic Acid Res. 29, 1772-1780; Dance et al., 2000 Nucleic Acids Res. 28, 424-9), the E. coli CDA was PCR amplified from genomic DNA and subcloned for yeast expression as described below. In order to address the question of whether or not the proposed homology model for APOBEC-1 (above) was feasible in terms of the overall three-dimensional fold and catalytic activity, a series of Cdd1 chimeras were assembled by fusing together two Cdd1 polypeptide chains joined by a linker. The 5′ monomers containing the appropriate C-terminal APOBEC-1 or E. coli 19 amino acid linker were amplified and subcloned as described above. The amino terminally foreshortened C-terminal monomer (missing helix α1 based upon homology modeling) was PCR amplified using the wild type or Glu63 to Ala Cdd1 template and ligated as an EcoRI/XhoI fragment to the appropriate 5′ monomer in pET28a. The linking EcoRI site was mutagenized to restore the reading frame of the Cdd1 chimeras. All Cdd1 monomer and chimeric cDNAs were amplified using Cdd1 specific primers and subcloned via EcoRI and XbaI sites into a modified pYES2.0 vector to allow galactose regulated expression of an HA-epitope tagged protein in yeast for Western analysis. Cdd1 mutants and chimeric proteins were expressed and purified essentially as described above. The results of editing in the context of the yeast system established for APOBEC-1 and Cdd1 (Dance et al 2001 Nucleic Acid Res. 29, 1772-1780; Dance et al., 2000 Nucleic Acids Res. 28, 424-9) are summarized in FIG. 13.

In the context of late log phase growth in yeast with galactose feeding, overexpressed Cdd1 is capable of C to U specific editing of reporter apoB mRNA at site C₆₆₆₆ at a level of 6.7%, which is ˜10× times greater than the negative control (FIG. 13, empty vector—compare lanes 1 and 2, above). In contrast, the CDA from E. coli (equivalent to PDB entry 1AF2) is incapable of editing on the reporter substrate (FIG. 13, lane 3). Similarly, the active site mutants E61A and G137A abolish detectable Cdd1 activity (FIG. 13, lanes 4 and 5). Likewise, the addition of the E. coli linker sequence (FIG. 13, lane 6) impairs editing function as well. In a series of chimeric constructs in which the Cdd1 tetramer was converted into a molecular dimer, the chimeric molecule appears functional, as long as an amino acid linker of 7-8 amino acids is used to join the respective Cdd1 subunits (FIG. 13, Right Panel lanes 1-4). However, when the longer E. coli linker is used to join Cdd1 monomers, there is no detectable activity on the reporter substrate, although the chimeric protein is expressed (FIG. 13, Western blot). Paradoxically, when conserved Gly residues of the APOBEC-1 linker (130 and 138) are mutated to Ala, the chimeric enzyme is still active (FIG. 13, lanes 3 and 4 of right panel). This shows that these components are not an important part of the linker flexibility, or that the new chimera adopts a different fold in this region compared to that of the pyrimidine metabolism enzymes. Indeed, the ARP models suggest a re-structuring of the active site linker that makes the entire region spanning from 130 to 142 (human APOBEC-1 numbering) flexible in a manner that moves to accommodate large polymeric substrates such as RNA or DNA (See AID active site model bound to DNA 9-mer BELOW). Additional evidence of the importance of the linker sequence comes from mutagenesis on rat APOBEC-1 (highly homologous to human). When the 8 amino acid linker sequence of rat APOBEC-1 is replaced with the first 8 amino acids of the E. coli linker, the APOBEC-1 construct is unable to edit reporter apoB mRNA in the human hepatoma cell line HepG2 (Navaratnam, N. et al. (1998) JMB 275:695-714; Chester et al., 2003 EMBO J. 22, 3971-3982).

### (6) Editing Activity

Editing activity for wild type and mutant constructs of scCdd1 were measured as described previously and in the following examples. cl (7) Results

The hidden Markov modeling software SAM. was trained with CDD1, APOBEC1, APOBEC2, AID and phorbolin 1. This identified APOBEC3A, 3B, 3C, 3E, 3F, 3G, XP₁₃ 092919, PHB1, XP₁₃ 115170/XP₁₃ 062365.

PHI-BLAST, using the target pattern H[VA]-E-x-x-F-(x)19-[I/V]-[T/V]-[W/C]-x-x-S-W-[ST]-P-C-x-x-C (SEQ ID NO: 60) limited the search more and misses only the 3B (Phorbolin 2) variant AAD00089 in which a single codon change GAC/T (SEQ ID NO: 63) to GAA/G (SEQ ID NO: 64) changes the ZDD center HxE to HxA. This is either a sequencing error or a significant SNP for psoriasis.

[HC]-x-E-x-x-F-x(19,30)-P-C-x(2,4)-C (SEQ ID NO: 61) yields the usual suspects for human. There are a couple of novel deaminases with motif HPE . . . SPC . . . C. Also identifies a mouse gene homologous to hu APOBEC3G (CEM15). On Chrom. 15, position 15E2. This is highly homologous to APOBEC3B, D+E, G. There are 9 exons. Both ZDDs fall in their own exons. On the mouse gene, the start of the linker is an exon junction.

The multiple sequence alignment results are shown below in Table 4.

[image]

[image]

[image]

The TBLASTN results are shown in Table 5:

TABLE 5

>gi|20902839|ref|XP 122858.1|
(XM_122858) similar to hypothetical protein,
MGC:7002; hypothetical protein
MGC7002 [Mus musculus]
Length = 429
Score = 180 bits (457), Expect = 1e − 44
Identities = 47/171 (27%), Positives = 75/171 (43%), Gaps = 9/171 (5%)

| | | | |
|---|---|---|---|
| Query: 14 | LRRRIEPWEFDVFYDP---RELRKEACLLYEIKW---GMSRKIWRSSGKNTTN-HVEVNF | 66 | |
| | +R I F + + RK+ L YE+ + KN N H E+ F | | |
| Sbjct: 17 | IRNLISQETFKFHFKNLGYAKGRKDTFLCYEVTRKDCDSPVSLHHGVFKNKDNIHAEICF | 76 | |
| Query: 67 | IKKFTS--ERDFHPSISCSITWFLSWSPCWECSQAIREFLSRHPGVTLVIYVARLFWHMD | 124 | |
| | + F + P ITW++SWSPC+EC++ I FL+ H ++L I+ +RL+ D | | |
| Sbjct: 77 | LYWFHDKVLKVLSPREEFKITWYMSWSPCFECAEQIVRFLATHHNLSLDIFSSRLYNVQD | 136 | |
| Query: 125 | QQNRQGLRDLVNSGVTIQIMRASEYYHCWRNFVNYPPGDEAHWPQYPPLWM | 175 | (SEQ ID NO: 14) |
| | + +Q L LV G + M E+ CW+ FV+ W + + | | |
| Sbjct: 137 | PETQQNLCRLVQEGAQVAAMDLYEFKKCWKKFVDNGGRRFRPWKRLLTNFR | 187 | (SEQ ID NO: 15) |

Score = 121 bits (303), Expect = 8e − 27
Identities = 41/168 (24%), Positives = 71/168 (41%), Gaps = 17/168 (10%)

| | | | |
|---|---|---|---|
| Query: 16 | RRIEP---WEFDVFYDPR-------ELRKEACLLYEIKWGMSRKIWRS--SGKNTTNHVE | 63 | |
| | RR++P EF + + R + L Y+++ + + + H E | | |
| Sbjct: 231 | RRMDPLSEEEFYSQFYNQRVKHLCYYHRMKPYLCYQLEQFNGQAPLKGCLLSEKGKQHAE | 290 | |
| Query: 64 | VNFIKKFTSERDFHPSISCSITWFLSWSPCWECSQAIREFLSRHPGVTLVIYVARLFWHM | 123 | |
| | + F+ K +IT +L+WSPC C+ + F P + L IY +RL++H | | |
| Sbjct: 291 | ILFLDKI----RSMELSQVTITCYLTWSPCPNCAWQLAAFKRDRPDLILHIYTSRLYFHW | 346 | |
| Query: 124 | DQQNRQGLRDLVNSGVTIQIMRASEYYHCWRNFVNYPPGDEAHWPQYP | 171 | (SEQ ID NO: 16) |
| | + ++GL L SG+ + +M ++ CW NFV P W | | |
| Sbjct: 347 | KRPFQKGLCSLWQSGILVDVMDLPQFTDCWTNFV-NPKRPFWPWKGLE | 393 | (SEQ ID NO: 17) |

>gi|13384970|ref|NP 084531.1|
(NM_030255) hypothetical protein,
MGC:7002; hypothetical protein
MGC7002 [Mus musculus]
gi|13097063|gb|AAH03314.1|AAH03314
(BC003314) Unknown (protein for MGC:7002) [Mus musculus]
Length = 429
Score = 176 bits (446), Expect = 3e − 43
Identities = 47/171 (27%), Positives = 75/171 (43%), Gaps = 9/171 (5%)

| | | | |
|---|---|---|---|
| Query: 14 | LRRRIEPWEFDVFYDPREL---RKEACLLYEIKW---GMSRKIWRSSGKNTTN-HVEVNF | 66 | |
| | +R I F + RK+ L YE+ + KN N H E+ F | | |
| Sbjct: 17 | IRNLISQETFKFHFKNLRYAIDRKDTFLCYEVTRKDCDSPVSLHHGVFKNKDNIHAEICF | 76 | |
| Query: 67 | IKKFTS--ERDFHPSISCSITWFLSWSPCWECSQAIREFLSRHPGVTLVIYVARLFWMMD | 124 | |
| | + F + P ITW++SWSPC+EC++ + FL+ H ++L I+ +RL+ D | | |
| Sbjct: 77 | LYWFHDKVLKVLSPREEFKITWYMSWSPCFECAEQVLRFLATHHNLSLDIFSSRLYNIRD | 136 | |
| Query: 125 | QQNRQGLRDLVNSGVTIQIMRASEYYHCWRNFVNYPPGDEAHWPQYPPLWM | 175 | (SEQ ID NO: 18) |
| | +N+Q L LV G + M E+ CW+ FV+ W + + | | |
| Sbjct: 137 | PENQQNLCRLVQEGAQVAAMDLYEFKKCWKKFVDNGGRRFRPWKKLLTNFR | 187 | (SEQ ID NO: 19) |

Score = 118 bits (297), Expect = 5e − 26
Identities = 37/165 (22%), Positives = 67/165 (40%), Gaps = 14/165 (8%)

| | | | |
|---|---|---|---|
| Query: 16 | RRIEPWEFDVFYDPRELRK-------EACLLYEIKWGMSRKIWRS--SGKNTTNHVEVNF | 66 | |
| | + EF + + ++ + L Y+++ + + + H E+ F | | |
| Sbjct: 234 | HLLSEEEFYSQFYNQRVKHLCYYHGMKPYLCYQLEQFNGQAPLKGCLLSEKGKQHAEILF | 293 | |
| Query: 67 | IKKFTSERDFHPSISCSITWFLSWSPCWECSQAIREFLSRHPGVTLVIYVARLFWHMDQQ | 126 | |
| | + K IT +L+WSPC C+ + F P + L IY +RL++H + | | |
| Sbjct: 294 | LDKI----RSMELSQVIITCYLTWSPCPNCAWQLAAFKRDRPDLILHIYTSRLYFHWKRP | 349 | |
| Query: 127 | NRQGLRDLVNSGVTIQIMRASEYYHCWRNFVNYPPGDEAHWPQYP | 171 | (SEQ ID NO: 20) |
| | ++GL L SG+ + +M ++ CW NFV P W | | |
| Sbjct: 350 | FQKGLCSLWQSGILVDVMDLPQFTDCWTNFV-NPKRPFWPWKGLE | 393 | (SEQ ID NO: 21) |

The BLAST alignment is shown in Table 6:

TABLE 6

| | | |
|---|---|---|
| Sequences producing significant alignments: | Score (bits) | E Value |
| ref\|NW_000106.1\|Mm15_WIFeb01_286 Mus musculus WGS supercont . . . | 1156 | 0.0 |
| Alignments | | |
| >ref\|NW_000106.1\|Mm15_WIFeb01_286 Mus musculus WGS supercontig | | |
| Mm15_WIFeb01_286 | | |
| Length = 65562851 | | |
| Score = 1156 bits (601), Expect = 0.0 | | |
| Identities = 615/621 (99%), Gaps = 4/621 (0%) | | |
| Strand = Plus / Plus | | |

| | | | |
|---|---|---|---|
| Query: 1223 | agtcctggggtctgcaagatttggtgaatgactttggaaacctacagcttggacccccga | 1282 | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: 41563126 | agtcctggggtctgcaagatttggtgaatgactttggaaacctacagcttggacccccga | 41563185 | |
| Query: 1283 | tgtcttgagaggcaagaagagattcaagaaggtcttttggtgacccccccacccaacccc | 1342 | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: 41563186 | tgtcttgagaggcaagaagagattcaagaaggtcttttggtgacccccccacccaacccc | 41563245 | |
| Query: 1343 | aagtctaggagaccttttgttctcccgtttgtttccccttttgttttatcttttgttgtt | 1402 | |
| | \|\|\|\|\|\|\|\|\|\|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\| | | |
| Sbjct: 41563246 | aagtctaggagaccttttgttctcctgtttgtttccccttttgttttatcttttgttgtt | 41563305 | |
| Query: 1403 | ttgctttgttttgaagacagagtctcactgggtagcttgctactctggaactcactacta | 1462 | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: 41563306 | ttgctttgttttgaagacagagtctcactgggtagcttgctactctggaactcactacta | 41563365 | |
| Query: 1463 | gactaagctggccttaaactctaaaatccacctgccaatgccttctgagagccaggctta | 1522 | |
| | \|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\|\|\|\|\|\|\|\| | | |
| Sbjct: 41563366 | gactaagctggccttaaactctaaaatccacctgccagtgccttctgagagccaggctta | 41563425 | |
| Query: 1523 | aggtgtgcgctgcccactcccagccttaacccactgtggcttttccttcctctttctttt | 1582 | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: 41563426 | aggtgtgcgctgcccactcccagccttaacccactgtggcttttccttcctctttctttt | 41563485 | |
| Query: 1583 | attatctttttatctcccctcaccctcccgccatcaataggtacttaattttgtacttga | 1642 | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: 41563486 | attatctttttatctcccctcaccctcccgccatcaataggtacttaattttgtacttga | 41563545 | |
| Query: 1643 | aatttttaagttgggccaggcatggtggagcagcgtgcctctaatcgcaggcaggaggat | 1702 | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: 41563546 | aatttttaagttgggccaggcatggtggagcagcgtgcctctaatcgcaggcaggaggat | 41563605 | |
| Query: 1703 | ttccacgagcttgaggctagcctgatctacatagtgggctccaggacagccagaactaca | 1762 | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: 41563606 | ttccacgagcttgaggctagcctgatctacatagtgggctccaggacagccagaactaca | 41563665 | |
| Query: 1763 | cagagaccctgtctcaaaaataaatttagatagataaatacataaataaataaatggaag | 1822 | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\| | | |
| Sbjct: 41563666 | cagagaccctgtctcaaaaataaatttagatagataaatacataaataaat----ggaag | 41563721 | |
| Query: 1823 | aagtcaaagaaagaaagacaa | 1843 | (SEQ ID NO: 22) |
| | \|\|\|\|\|\|\|\|\|\|\|\|\|\|\|\|\|\|\|\|\| | | |
| Sbjct: 41563722 | aagtcaaagaaagaaagacaa | 41563742 | (SEQ ID NO: 23) |

Score = 508 bits (264), Expect = e − 141
Identities = 274/279 (98%)
Strand = Plus / Plus

| | | | |
|---|---|---|---|
| Query: 200 | aggacaacatccacgctgaaatctgctttttatactggttccatgacaaagtactgaaag | 259 | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: 41553517 | aggacaacatccacgctgaaatctgctttttatactggttccatgacaaagtactgaaag | 41553576 | |
| Query: 260 | tgctgtctccgagagaagagttcaagatcacctggtatatgtcctggagcccctgtttcg | 319 | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: 41553577 | tgctgtctccgagagaagagttcaagatcacctggtatatgtcctggagcccctgtttcg | 41553636 | |
| Query: 320 | aatgtgcagagcaggtactaaggttcctggctacacaccacaacctgagcctggacatct | 379 | |
| | \|\|\|\|\|\|\|\|\|\|\|\|\|\| \|\| \|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: 41553637 | aatgtgcagagcagatagtaaggttcctggctacacaccacaacctgagcctggacatct | 41553696 | |
| Query: 380 | tcagctcccgcctctacaacatacgggacccagaaaaccagcagaatctttgcaggctgg | 439 | |
| | \|\|\|\|\|\|\|\|\|\|\|\|\|\|\|\|\|\|\|\| \|\|\| \|\|\|\|\|\|\|\|\|\|\| \|\|\|\|\|\|\|\|\|\|\|\|\|\|\|\|\|\|\|\|\|\|\| | | |
| Sbjct: 41553697 | tcagctcccgcctctacaacgtacaggacccagaaacccagcagaatctttgcaggctgg | 41553756 | |
| Query: 440 | ttcaggaaggagcccaggtggctgccatggacctatacg | 478 | (SEQ ID NO: 24) |
| | \|\|\|\|\|\|\|\|\|\|\|\|\|\|\|\|\|\|\|\|\|\|\|\|\|\|\|\|\|\|\|\|\|\|\|\|\|\|\| | | |
| Sbjct: 41553757 | ttcaggaaggagcccaggtggctgccatggacctatacg | 41553795 | (SEQ ID NO: 25) |

Score = 502 bits (261), Expect = e − 139
Identities = 263/264 (99%)
Strand = Plus / Plus

| | | | |
|---|---|---|---|
| Query: 848 | agaaaggcaaacagcatgcagaaatcctcttccttgataagattcggtccatggagctga | 907 | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: 41562163 | agaaaggcaaacagcatgcagaaatcctcttccttgataagattcggtccatggagctga | 41562222 | |
| Query: 908 | gccaagtgataatcacctgctacctcacctggagcccctgcccaaactgtgcctggcaac | 967 | |
| | \|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: 41562223 | gccaagtgacaatcacctgctacctcacctggagcccctgcccaaactgtgcctggcaac | 41562282 | |
| Query: 968 | tggcggcattcaaaagggatcgtccagatctaattctgcatatctacacctcccgcctgt | 1027 | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: 41562283 | tggcggcattcaaaagggatcgtccagatctaattctgcatatctacacctcccgcctgt | 41562342 | |
| Query: 1028 | atttccactggaagaggcccttccagaaggggctgtgttctctgtggcaatcagggatcc | 1087 | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: 41562343 | atttccactggaagaggcccttccagaaggggctgtgttctctgtggcaatcagggatcc | 41562402 | |
| Query: 1088 | tggtggacgtcatggacctcccac | 1111 | (SEQ ID NO: 26) |
| | \|\|\|\|\|\|\|\|\|\|\|\|\|\|\|\|\|\|\|\|\|\|\|\|\| | | |
| Sbjct: 41562403 | tggtggacgtcatggacctcccac | 41562426 | (SEQ ID NO: 27) |

Score = 283 bits (147), Expect = 2e − 73
Identities = 155/159 (97%)
Strand = Plus / Plus

| | | | |
|---|---|---|---|
| Query: 691 | aggcgagtgcacctgctaagtgaagaggaattttactcgcagttttacaaccaacgagtc | 750 | |
| | \|\|\|\|\|\| \|\| \|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: 41561266 | aggcgaatggacccgctaagtgaagaggaattttactcgcagttttacaaccaacgagtc | 41561325 | |
| Query: 751 | aagcatctctgctactaccacggcatgaagccctatctatgctaccagctggagcagttc | 810 | |
| | \|\|\|\|\|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\| | | |
| Sbjct: 41561326 | aagcatctctgctactaccaccgcatgaagccctatctatgctaccagctggagcagttc | 41561385 | |
| Query: 811 | aatggccaagcgccactcaaaggctgcctgctaagcgag | 849 | (SEQ ID NO: 28) |
| | \|\|\|\|\|\|\|\|\|\|\|\|\|\|\|\|\|\|\|\|\|\|\|\|\|\|\|\|\|\|\|\|\|\|\|\|\|\|\| | | |
| Sbjct: 41561386 | aatggccaagcgccactcaaaggctgcctgctaagcgag | 41561424 | (SEQ ID NO: 29) |

Score = 269 bits (140), Expect = 3e − 69
Identities = 148/152 (97%)
Strand = Plus / Plus

| | | |
|---|---|---|
| Query: 51 | cagaaacctgatatctcaagaaacattcaaattccactttaagaacctacgctatgccat | 110 |
| | \|\|\|\|\|\|\|\|\|\|\|\|\|\|\|\|\|\|\|\|\|\|\|\|\|\|\|\|\|\| \|\|\|\|\|\|\|\|\|\|\|\|\|\|\|\|\|\| \|\|\|\|\|\|\|\|\| | |
| Sbjct: 41551231 | cagaaacctgatatctcaagaaacattcaagttccactttaagaacctaggctatgccaa | 41551290 |
| Query: 111 | agaccggaaagataccttcttgtgctatgaagtgactagaaaggactgcgattcacccgt | 170 |
| | \|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 41551291 | aggccggaaagataccttcttgtgctatgaagtgactagaaaggactgcgattcacccgt | 41551350 |
| Query: 171 | ctcccttcaccatggggtctttaagaacaagg | 202 |
| | \|\|\|\|\|\|\|\|\|\|\|\|\|\|\|\|\|\|\|\|\|\|\|\|\|\|\|\|\|\|\|\| | |
| Sbjct: 41551351 | ctcccttcaccatggggtctttaagaacaagg | 41551382 |

Score = 212 bits (110), Expect = 6e − 52
Identities = 114/116 (98%)
Strand = Plus / Plus

| | | | |
|---|---|---|---|
| Query: 478 | gaatttaaaaagtgttggaagaagtttgtggacaatggcggcaggcgattcaggccttgg | 537 | |
| | \|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\|\|\|\|\|\|\| | | |
| Sbjct: 41553934 | gaatttaaaaagtgttggaagaagtttgtggacaatggtggcaggcgattcaggccttgg | 41553993 | |
| Query: 538 | aaaaaactgcttacaaattttagataccaggattctaagcttcaggagattctgag | 593 | (SEQ ID NO: 30) |
| | \|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: 41553994 | aaaagactgcttacaaattttagataccaggattctaagcttcaggagattctgag | 41554049 | (SEQ ID NO: 31) |

Score = 212 bits (110), Expect = 6e − 52
Identities = 112/113 (99%)
Strand = Plus / Plus

| | | |
|---|---|---|
| Query: 1112 | agtttactgactgctggacaaactttgtgaacccgaaaaggccgttttggccatggaaag | 1171 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 41562675 | agtttactgactgctggacaaactttgtgaacccgaaaaggccgttttggccatggaaag | 41562734 |
| Query: 1172 | gattggagataatcagcaggcgcacacaaaggcggctccacaggatcaaggag | 1224 |
| | \|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\| | |
| Sbjct: 41562735 | gattggagataatcagcaggcgcacacaaaggcggctccgcaggatcaaggag | 41562787 |

Score = 187 bits (97), Expect = 2e − 44
Identities =+00 103/106 (97%)
Strand = Plus / Plus

| | | | |
|---|---|---|---|
| Query: 592 | agaccttgctacatcccggtcccttccagctcttcatccactctgtcaaatatctgtcta | 651 | |
| | \|\|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: 41554842 | agaccttgctacatctcggtcccttccagctcttcatccactctgtcaaatatctgtcta | 41554901 | |
| Query: 652 | acaaaaggtctcccagagacgaggttctgcgtggagggcaggcgag | 697 | (SEQ ID NO: 32) |
| | \|\|\|\|\|\|\|\|\|\|\|\|\|\|\|\|\|\|\|\|\|\|\|\|\|\|\|\|\| \|\|\|\|\|\|\|\|\|\|\|\| \|\|\| | | |
| Sbjct: 41554902 | acaaaaggtctcccagagacgaggttctgggtggagggcaggtgag | 41554947 | (SEQ ID NO: 33) |

Score = 102 bits (53), Expect = 6e − 19
Identities = 53/53 (100%)
Strand = Plus / Plus

| | | | |
|---|---|---|---|
| Query: 1 | atgggaccattctgtctgggatgcagccatcgcaaatgctattcaccgatcag | 53 | SEQ ID NO: 34) |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: 41548340 | atgggaccattctgtctgggatgcagccatcgcaaatgctattcaccgatcag | 41548392 | (SEQ ID NO: 35) |

### 3. Example 3

### a) Experimental

All plasmids were constructed by standard recombinant DNA methods and verified by DNA sequencing. The intervening sequence (IVS)-apoB construct has been described previously (Sowden, M., et al. (1996) RNA 2, 274-288) Mutation of 6 bp at the 5′ splice donor sequence, including the intronic GU dinucleotide (IVS-Δ5′apoB) and deletion of 20 bp encompassing the 3′ splice acceptor and polypyrimidine tract sequences (IVS-Δ3′apoB), was accomplished by ‘runaround’ PCR using primers that included an XhoI site to facilitate subsequent re-ligation of the PCR product (Fisher, C. L. et al. (1997) BioTechniques 23, 570-574.). IVS-Δ3′5′ apoB was created by ligation of the appropriate halves of the above molecules.

McArdle RH7777 cells were maintained as previously described (Sowden, M. P. et al., (1996) J. Biol. Chem. 271:3011-3017.) and transfected in six-well clusters with 2 μg of DNA using lipofectAMINE® (Gibco BRL) according to the manufacturer's recommendations. RNAs were harvested 48 h post-transfection in TriReagent (Molecular Research Center, Cincinnati, Ohio, U.S.A.) and subjected to reverse-transcriptase (RT)-PCR for amplification of intron-containing or exonic apoB specific transcripts using appropriate PCR primers as previously described (Sowden, M., et al. (1996) RNA 2, 274-288) and outlined in the Figure legends. Editing efficiencies were determined by poisoned-primer-extension assay on purified PCR products (Sowden, M., et al. (1996) RNA 2, 274-288) and quantified by analysis on a Phosphorlmager (model 425E; Molecular Dynamics).

The poisoned-primer-extension assay relies on the annealing of a ³²P-end-labelled primer 3′ of the editing site to the heat-denatured single-stranded PCR product. Extension of this primer using RT in the presence of dATP, dCTP, dTTP and dideoxy (dd)-GTP produces an extension product eight nucleotides longer if the cytidine has not been edited (CAA in the Figures); that is, incorporation of ddGTP causes chain termination. If editing has created a uridine, then primer extension continues a further 11 nucleotides to the next 5′ cytidine, where chain termination then occurs (UAA in the Figures). Quantification of the level of editing is accurately determined using laser scanning densitometry. The linear exposure range of the PhosphorImager screen is sufficiently great to permit precise determination of low counts in the UAA bands whilst the high levels of counts in the CAA band remain in the linear range. Editing percentages were calculated as the counts in the UAA band divided by the total counts in the CAA plus UAA bands times 100. This assay has a lower level of detection of 0.1% editing and remains linear up to 99.5% and is independent, between 1 ng and 500 ng, of the total amount of template PCR product used (M. P. Sowden, unpublished work).

Rev complementation/editing assays (Taagepera, S., et al. (1998) Proc. Natl. Acad. Sci. U.S.A. 95:7457-7462.) were performed in duplicate in McArdle cells seeded in six-well clusters. Briefly, a total of 2 μg of DNA, comprising 1 μg of reporter DNA, 0.75 μg of transactivator DNA (pRc/CMV vector or a nucleocytoplasmic shuttling competent Rev-Rex fusion; a gift of Dr Thomas J. Hope, Infectious Disease Laboratory, Salk Institute for Biological Studies, La Jolla, Calif., U.S.A.) and 0.25 μg of pRSV-β-galactosidase [internal control for chloramphenicol acetyl-transferase (CAT) assays] were introduced into McArdle cells using lipofectAMINE® as described above. Cells were harvested at 48 h post-transfection, protein extracts prepared by freeze-thawing, and β-gal (Sowden, M. P., et al. (1989) Nucleic Acids Res. 17:2959-2972) and CAT (Neumann, J. R., et al., (1987) Bio Techniques. 5:444-448.) assays performed as previously described. All extracts were normalized for b-gal activity. Parallel transfections were harvested for RNA preparation and RT-PCR amplification of the apoB RNA. Editing efficiencies were quantified as described above.

### b) Results

### (1) Introns Interfere with Editing

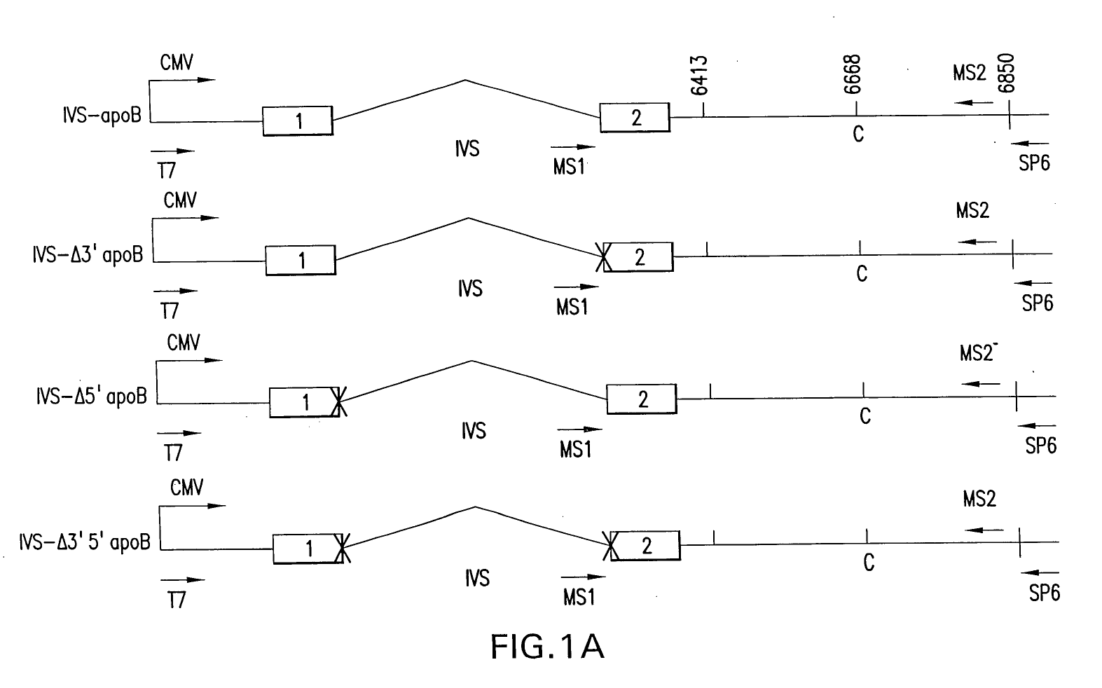

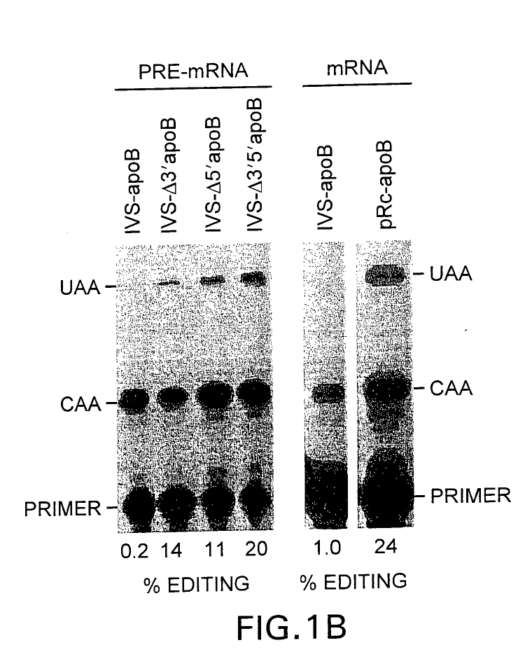

Previous studies demonstrated that the editing efficiency of apoB RNA was dramatically reduced when an intron was placed ≦350 nt 5′ or 3′ of the target cytidine (Sowden, M., et al. (1996) RNA 2, 274-288). To provide proof that it was specifically RNA splicing and/or spliceosome assembly that had affected editing efficiency, splicing-competent and splicing-defective RNA transcripts were evaluated for their ability to support RNA editing in transfected McArdle rat hepatoma cells. The apoB pre-mRNA reporter construct contained an abbreviated splicing cassette from the adenovirus late leader sequence fused to 450 nt of wild-type apoB mRNA (FIG. 1A). Unspliced pre-mRNA and spliced mRNA were amplified from total cellular McArdle cell mRNA using the MS1/MS2 and SP6/T7 amplimer pairs respectively (FIG. 1A). Consistent with previous results, the splicing cassette impaired the ability of the IVS-apoB RNA transcript to be edited, either before (pre-mRNA) or after (mRNA) it was spliced relative to a control transcript (pRc-apoB) that contained only apoB sequence (FIG. 1B). These results corroborate previous findings suggesting that there is a window of opportunity for editing apoB mRNA in the nucleus and that no further editing occurs in the cytoplasm of wild-type hepatic cells. Specifically, recently published subcellular-fractionation studies have shown that the low level of editing measured on this transcript as mRNA (1%) occurred while the RNA was still in the nucleus (Yang, Y., et al. (2000) J. Biol. Chem. 275: 22663-22669.).

Deletion of the polypyrimidine tract/branch point sequences and the 3′ splice acceptor site in the IVS-Δ3′apoB transcript (FIG. 1A) ablated the ability of this pre-mRNA to be spliced, as the SP6/T7 amplimer pair yielded only PCR products indicative of unspliced transcripts (results not shown). The editing efficiency of this splicing-defective construct was higher than that of IVS-apoB (14%, S.E.M=1.0%; FIG. 1B). The IVS-Δ5′apoB transcript was also defective in splicing owing to deletion of the 5′ splice donor sequence (the SP6/T7 amplimer pair failed to yield PCR products corresponding to spliced RNA; results not shown), and this RNA also demonstrated markedly elevated editing compared with IVS-apoB (11%, S.E.M.=0.1%; FIG. 1B). The double-splice-site mutant IVSD3′5′apoB (FIG. 1A) had an editing efficiency higher than either of the single-site mutants (20%, S.E.M.=0.2%) and equivalent to the intron lacking RNA transcript, pRc-apoB (24%, S.E.M.=0.2%; FIG. 1B). These results indicated that it is the assembly of a fully functional spliceosome and/or RNA splicing that impedes editosome assembly and/or function, and that both 5′ and 3′ splicing signals contribute to the inhibitory effect.

Each of the constructs in FIG. 1 generated pre-mRNA transcripts of equivalent length, but the presence of active or inactive introns might influence expression levels of the resultant mRNAs. However, it was previously reported that the expression level of a given apoB transcript did not affect its editing efficiency (Sowden, M., et al. (1996) RNA 2, 274-288). Moreover, there was no competition between the editing efficiencies of exogenous and endogenous apoB transcripts, indicating that editing factors were not made to be rate-limiting by the increased concentration of apoB editing sites. These facts underscore the significance of the intron and RNA splicing on the regulation of editing efficiency.

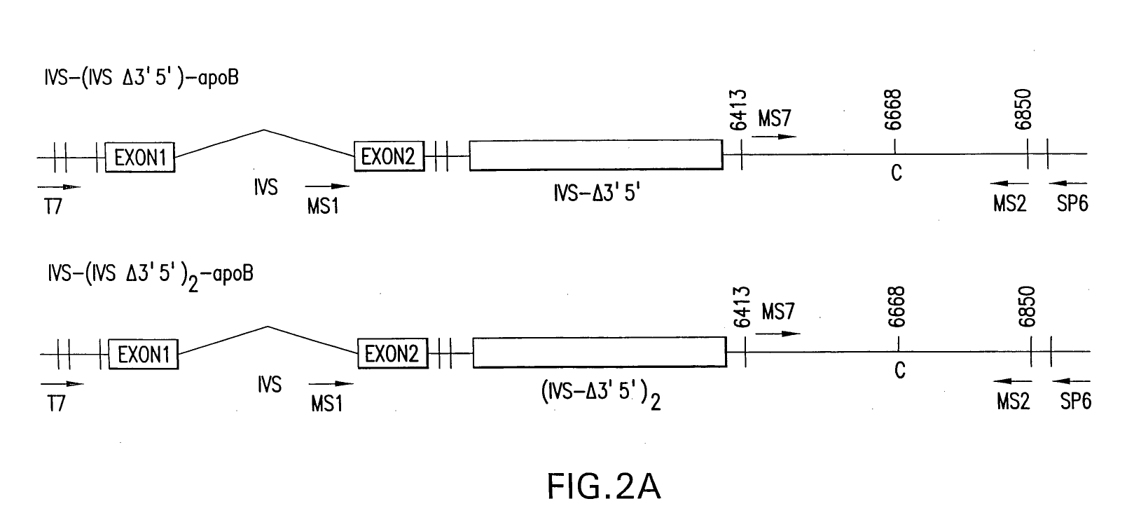

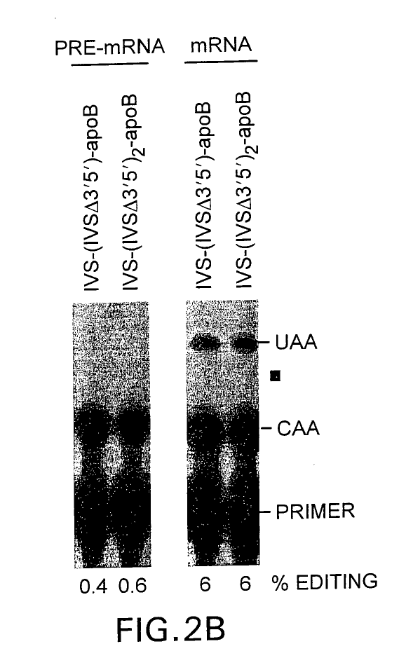

In human apoB mRNA, C⁶⁶⁶⁶ is located in the middle of the 7.5 kb exon 26, significantly further from a 5′ or 3′ intron than in the chimeric constructs described above. Therefore it was evaluated whether the proximity of the splice donor and acceptor sites to the tripartite motif affected editing efficiency. Insertion of a monomer or a dimer of the splicing-defective intron cassette (IVS Δ3′5′) increased the distance between the active intron and the editing site by 425 and 850 nt respectively (FIG. 2A). This increased the effective size of the chimeric exon to nearly 1 kb or 1.4 kb respectively; the average size of an internal exon being only 200-300 nt in mammals (Robberson, B. L., et al (1990) Mol. Cell. Biol. 10:1084-1094.).

ApoB pre-mRNA was amplified from each transcript expressed in McArdle cells using the MS7/MS2 amplimers and nesting with the MS2/MS3 amplimer pair. The sequence of primer MS7 is unique to the fuictional intron sequence and thus ensured amplification of unspliced pre-mRNA. Barely detectable levels of editing were measured on both pre-mRNA transcripts. However, a 10-fold higher level of editing was observed upon the spliced mRNA of both transcripts (6.0%) (FIG. 2B), which is 6-fold higher than the spliced mRNA derived from IVS-apoB (FIG. 1B). This indicated that increasing the distance between the intron and the editing site alleviated, but was not completely capable of overcoming, the inhibitory effect of spliceosome assembly/RNA splicing on editing (i.e. compare 6 with 20% editing of IVS Δ3′5′apoB in FIG. 1).

### (2) The apoB Editing Site is not Efficiently used within an intron

A search of GenBank2 for apoB mooring-sequence similarities reveals numerous potential editing sites. However, many are located short distances from splice sites or within 5′ or 3′ untranslated regions or introns where the fuinctional consequence(s) of a cytidine-to-uridine editing event is unclear. The release of the entire human, mouse and rat genome sequences will likely reveal more mooring-sequence similarities, although their location in introns or exons may be uncertain until these genomes are annotated. In this regard, the results-indicated that mooring-sequence-dependent editing sites may not be biologically active if they are positioned too close to splice junctions.

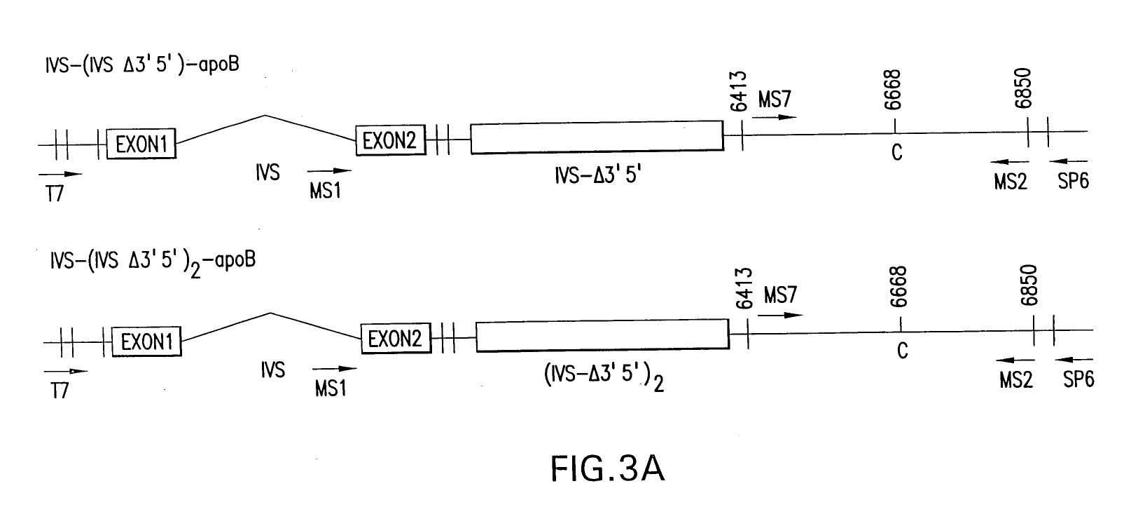

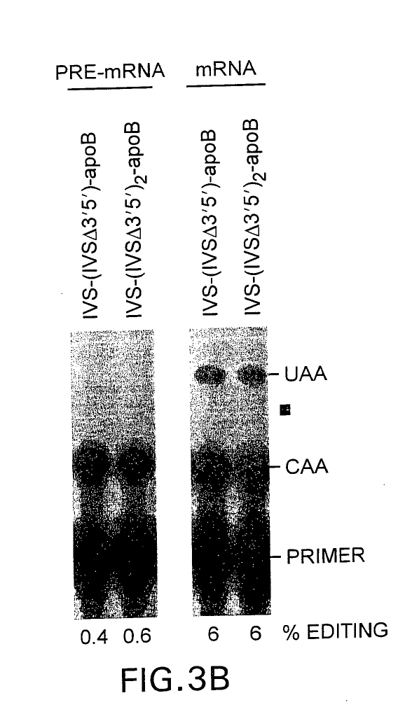

In an attempt to be able to predict functional cytidine-to-uridine editing sites from these transcriptomes, it was investigated whether the apoB editing site is recognized when positioned within an intron. A 450 nt section of the apoB RNA transcript containing the editing site was placed within the intron of the adenovirus late leader sequence (IVS-apoB INT) and this construct was expressed in transfected McArdle cells. Pre-mRNA transcripts were amplified using the Ex1/Ex2 amplimers followed by nested PCR with the MS Δ5/MSΔ6 amplimer pair and were edited at an efficiency of 0.4% (FIG. 3B). Intron-containing transcripts were amplified using the MS Δ5/MS Δ6 amplimers followed by nested PCR with the MS2/MS3 amplimer pair and were edited at an efficiency of 0.5% (FIG. 3B). The use of the MS Δ5/MS Δ6 amplimer pair in the initial PCR would not distinguish between unspliced pre-mRNA or spliced-out lariat RNA, but given the rapid degradation of lariat RNA, it is unlikely that the amplified PCR products represent lariat RNA species. If, however, there were amplified lariat species present, the difference of 0.1% between intron-containing and unspliced pre-mRNA suggests that lariat RNAs containing apoB editing sites are not efficient editing substrates.

Mutation of the 5′ and 3′ splicing signals of the above construct to generate IVS-Δ3′5′apoB INT restored editing efficiency (20%; FIG. 3B) to a level equal to that of IVS-Δ3′5′apoB construct (20%; FIG. 1C). A minor additional primer extension product indicative of promiscuous editing was also apparent. These results support the hypothesis that pre-mRNA is not an effective substrate for cytidine-to-uridine editing and that this likely results from interference by spliceosome assembly/RNA splicing or potentially the rapid nuclear export of spliced mRNAs into the cytoplasm.

### (3) Blocking the Commitment of Transcripts to the Splicing Pathway Alleviates Splice-Site Inhibition of Editing

Most apoB mRNA editing substrate studies have employed cDNA transcripts which lack introns [(Sowden M. P., et al. (1998) Nucleic Acids Res. 26:1644-1652; Driscoll, D. M., et al. (1993) Mol. Cell. Biol. 13:7288-7294; Bostrom, K., et al. (1990) J. Biol. Chem. 265:22446-22452.)]. Wild-type apoB cDNA transcripts expressed in wild-type McArdle cells edit 2-3-fold more efficiently than the endogenous transcript (Sowden, M., et al. (1996) RNA 2, 274-288; Sowden M. P., et al. (1998) Nucleic Acids Res. 26:1644-1652.). It has been demonstrated that chimeric splicing-editing reporter RNAs (IVS-apoB) had low editing efficiency as nuclear transcripts, which did not change once spliced mRNAs had entered the cytoplasm (FIG. 1; (Yang, Y., et al. (2000) J. Biol. Chem. 275: 22663-22669.)). Hence the window of opportunity for a transcript to be edited in wild-type cells was confined to the nucleus, and when introns are proximal to the editing site, its utilization was impaired.

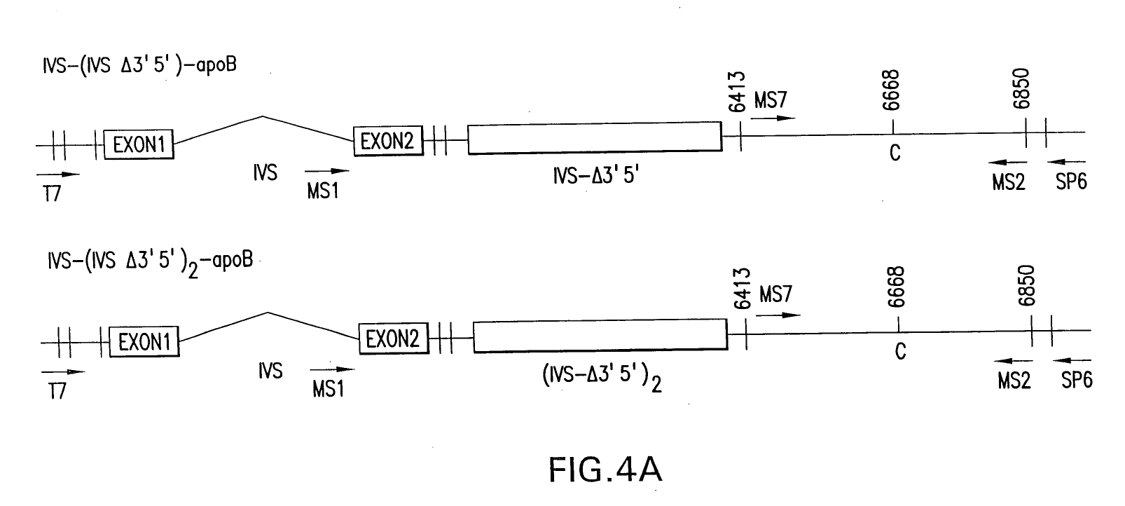

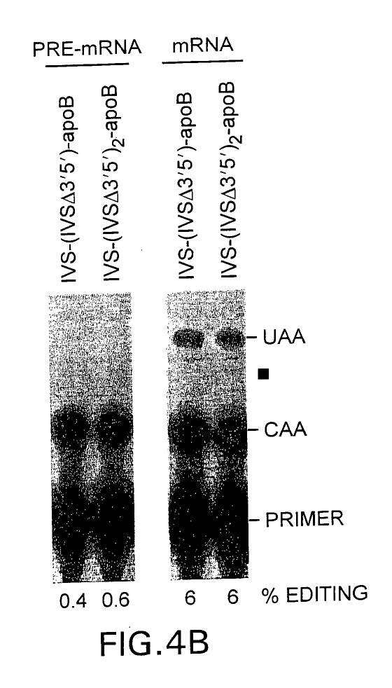

To investigate if spliceosome assembly was involved in the inhibition of editing, and by-passing the spliceosome assembly commitment step inhibition may be alleviated (in a manner similar to intron-less cDNA transcripts), the processes of RNA splicing and RNA nuclear export were separated by utilizing a modification of the Rev complementation assay that has been employed to identify HIV-1 Rev-like nuclear export sequences (Taagepera, S., et al. (1998) Proc. Natl. Acad. Sci. U.S.A. 95:7457-7462.). Rev functions, by interaction with an RRE, to export unspliced RNA out of the nucleus. A reporter plasmid was constructed which contained an intron interrupted by the CAT gene and a functional apoB RNA editing cassette (FIG. 4A). CAT activity could only be expressed if unspliced RNA was exported to the cytoplasm, a process wholly dependent upon an active Rev protein expressed from a co-transfected plasmid. In the presence of Rev, spliceosome assembly on the transcript does not occur and therefore should not interfere with the utilization of the apoB editing site contained with the intron.

McArdle cells were co-transfected with the modified reporter construct, together with either a control vector or a Rev expression vector. CAT activity was determined 48 h later (FIG. 4B). In the presence of the control vector, very low levels of CAT activity were expressed, presumed to be due to splicing and degradation of the CAT transcript as a lariat RNA. Expression of the Rev protein resulted in nuclear export of unspliced intronic RNA and translation of the CAT protein, as evident in the 7-fold higher level of CAT activity in these cell extracts. These findings demonstrated that, in McArdle cells, HIV-1 Rev protein successfully diverted RNAs from the spliceosome assembly pathway and transported them into the cytoplasm.

Total cellular RNA was harvested from parallel transfections, the apoB sequence amplified, and the editing efficiencies were determined (FIG. 4C). Consistent with the findings described above, editing of apoB RNA within an intron of the RRE construct in the absence of Rev expression was very low (‘intron+exon’ amplified with EF/MS2). However, the editing efficiency was enhanced 5-fold when the Rev protein was co-expressed. Given that editing in the cytoplasm has never been demonstrated in wild-type McArdle cells (Yang, Y., et al. (2000) J. Biol. Chem. 275: 22663-22669.), nor would it be driven by an increase in apoB RNA abundance in the cytoplasm (Sowden, M., et al. (1996) RNA 2, 274-288), it appears enhanced editing occurred in the nucleus as a consequence of pre-mRNAs by-passing commitment to the spliceosome assembly and/or RNA export pathways. Editing unspliced CAT-apoB chimeric RNAs in the cytoplasm necessitates the activation of cytoplasmically localized editing factors by Rev.

In addition to an enhanced editing efficiency, the unspliced CAT-apoB RNA was also promiscuously edited (additional primer extension stop labeled ‘1’, FIG. 4C). Promiscuous editing does not occur under physiological expression levels of APOBEC-1 in McArdle cells (Sowden, M., et al. (1996) RNA 2, 274-288; Sowden, M. P. et al., (1996) J. Biol. Chem. 271:3011-3017; Siddiqui, J. F., et al. (1999) Exp Cell Res. 252:154-164.), in rat tissues or under biological conditions where editing efficiencies are greater than 90%, e.g. rat intestine (Greeve, J., et al. (1993) J. Lipid Res. 34:1367-1383.). Nor does it occur when rat hepatic editing efficiencies are stimulated by metabolic or hormonal manipulations (Lau, P. P., et al. (1995) J. Lipid Res. 36:2069-2078; Baum, C. L. et al. (1990) J. Biol. Chem. 265: 19263-19270.). Promiscuous editing appears to be unique to cells in which APOBEC-1 has been artificially overexpressed (Sowden, M., et al. (1996) RNA 2, 274-288; Sowden, M. P. et al., (1996) J. Biol. Chem. 271:3011-3017; Siddiqui, J. F., et al. (1999) Exp Cell Res. 252:154-164.) and is observed under these conditions on both nuclear and cytoplasmic transcripts (Yang, Y., et al. (2000) J. Biol. Chem. 275: 22663-22669.). The results presented in FIGS. 3 and 4 are therefore the first demonstration of promiscuous editing in the nucleus without the exogenous over-expression of APOBEC-1.

### c) Discussion

ApoB mRNA editing, while conceptually a simple process of hydrolytic cytidine deamination to uridine (Johnson, D. F., et al. (1993) Biochem. Biophys. Res. Commun. 195:1204-1210.) has complexities in both the number of proteins involved and the cell biology involved in its regulation. It is well established that a sequence element consisting of three proximal components (enhancer, spacer and mooring sequence) comprise the cis-acting sequences required for efficient site-specific editing of C⁶⁶⁶⁶ in apoB mRNA Smith, H. C., et al (1991) Proc. Natl. Acad. Sci. U.S.A. 88:1489-1493; Backus, J. W., et al, (1992) Nucleic Acids Res. 20: 6007-6014; Smith, H. C. (1993) Semin. Cell. Biol. 4:267-278; Shah R. R., et al. (1991) J. Biol. Chem. 266:16301-16304; Backus, J. W., et al, (1991) Nucleic Acids Res. 19: 6781-6786; Driscoll, D. M., et al. (1993) Mol. Cell. Biol. 13: 7288-7294.). A multiple protein editosome catalyses and regulates editing of C⁶⁶⁶⁶ [Smith, H. C., et al. (1991) Proc. Natl. Acad. Sci. U.S.A. 88:1489-1493; Harris, S. G., et al. (1993) J. Biol. Chem. 268:7382-7392; Yang, Y., et al. (1997) J. Biol. Chem. 272: 27700-27706.). The components of the minimal editosome from defined in vitro system analyses are APOBEC-1 as a homodimeric cytidine deaminase (Lau, P. P., et al. (1994) Proc. Natl. Acad. Sci. U.S.A. 91:8522-8526.) bound to the auxiliary protein ACF/ASP that serves as the editing-site recognition factor through its mooring-sequence-selective RNA-binding activity (Mehta, A., et al. (2000) Mol. Cell. Biol. 20:1846-1854; Lellek, H., et al. (2000) J. Biol. Chem. 275:19848-19856.). Several other auxiliary protein candidates have also been described that had binding affinities for APOBEC-1 and/or apoB mRNA and that demonstrated the ability to modulate editing efficiency (Giannoni, F., et al. (1994) J. Biol. Chem. 269:5932-5936; Ymanaka, S., et al. (1994) J. Biol. Chem. 269:21725-21734; Yang, Y., et al. (1997) J. Biol. Chem. 272: 27700-27706; Lellek, H., et al. (2000) J. Biol. Chem. 275:19848-19856; Teng, B., et al. (1993) Science 260:1816-1819; Inui, Y., et al. (1994) J. Lipid Res. 35:1477-1489; Anant, S. G., et al. (1997) Nucleic Acids Symp. Ser. 36:115-118; Lau, P. P., et al. (1997) J. Biol. Chem. 272:1452-1455.). Although, under biological conditions, editing occurs only in the nucleus (Lau, P. P., et al. (1991) J. Biol. Chem. 266, 20550-20554; Yang, Y., et al. (2000) J. Biol. Chem. 275:22663-22669.), nuclear and cytoplasmic distributions have been described for both APOBEC-1 and ACF (Yang, Y., et al. (2000) J. Biol. Chem. 275:22663-22669; Yang, Y., et al. (1997) Proc. Natl. Acad. Sci. U.S.A. 94:13075-13080; Dance, G. S. C., et al. (2000) Nucleic Acids Res. 28:424-429.). Nuclear editing has been characterized as occurring coincident with, or immediately after, pre-mRNA splicing (Lau, P. P., et al. (1991) J. Biol. Chem. 266, 20550-20554; Yang, Y., et al. (2000) J. Biol. Chem. 275:22663-22669; Sowden, M., et al. (1996) RNA 2:274-288.). Prior to splicing, pre-mRNA was not efficiently edited (Lau, P. P., et al. (1991) J. Biol. Chem. 266, 20550-20554.). It was not apparent, given the size of exon 26 and the nature of the cis-acting RNA sequence requirements, why there was a lag in editing activity during pre-mRNA maturation. This question was addressed in studies indicating that spliceosome assembly and/or nuclear RNA export pathways regulate the utilization of cytidine-to-uridine editing sites.

In reporter RNA constructs, introns within 350-1000 nt of the apoB editing site suppressed editing efficiency. This inhibition was dependent on an active 5′ splice site and/or 3′ splice donor site and was partially alleviated after the reporter RNA had been spliced. This indicates that the process of spliceosome assembly functionally interfered with editosome assembly and/or function. This is supported by the distance dependence of this inhibition. When the splice sites were located more distal to the editing site, editing efficiencies were increased albeit not to levels seen on RNAs that do not contain introns. The gating hypothesis (Sowden, M., et al. (1996) RNA 2, 274-288) proposed that each apoB RNA had a temporal ‘window of opportunity’ to become edited during its splicing and export from the nucleus. In this model, factors involved in spliceosome and editosome assembly are thought to compete for access to the mRNA. Consequently it is predicted that there will be less steric hindrance between the spliceosome and the editosome, and editing efficiency will improve the more distal an intron is located relative to the editing site [e.g. IVS-(IVS Δ3′5′)-apoB or IVS-(IVS Δ3′5′)₂-apoB compared with IVS-apoB]. This phenomenon might explain the lower editing efficiency of native apoB editing prior to splicing, because the native editing site is only three times further away from the 5′ or 3′ splice junctions than that used in our reporter RNA constructs.

Importantly, these results have implications for the prediction of novel mooring-sequence-dependent RNA-editing sites. Not only is there a requirement for a target cytidine to be appropriately located upstream of a mooring sequence, but for efficient utilization, the editing site should not be in close proximity to an intron. Considering that the average size of an internal exon is only 200-300 nt in mammals (Robberson, B. L., et al. (1990) Mol. Cell. Biol. 10, 1084-1094.), it is highly unlikely that a significant amount of mooring-sequence-dependent editing will be observed in mRNAs with standard sized exons. In fact an analysis of the human, mouse and rat expressed-sequence-tag databases by Hidden Markov modeling has confirmed that the majority of mooring-sequence identities within coding sequences are located proximal to intron/exon junctions. An evaluation of select RNA transcripts revealed that they were in fact not edited. Related to these observations are results showing that editing sites located within introns were not inefficiently utilized. Taken together, the results support the hypothesis that spliceosome assembly and editosome assembly processes are communicating a temporal and spatial relationship that ultimately determines the efficiency of mooring-sequence-dependent editing. Consistent with this communication between the spliceosome and editosome is the finding that several proteins that have a role in RNA structure and/or splicing have also been implicated in RNA editing as auxiliary factors. These include hnRNP C, hnRNP D, APOBEC-1-binding protein (which has homology with hnRNP A and B) and KSRP, a protein involved in alternative splice site utilization (Lellek, H., et al. (2000) J. Biol. Chem. 275:19848-19856; Greeve, J., et al. (1998) J. Biol. Chem. 379:1063-1073; Anant, S. G., et al. (1997) Nucleic Acids Symp. Ser. 36:115-118; Lau, P. P., et al. (1997) J. Biol. Chem. 272:1452-1455.).

The promiscuous editing observed on IVS-Δ3′5′apoB INT was unexpected, given the nature of the transcript, i.e. a cDNA equivalent to IVS-Δ3′5′apoB in FIG. 1 on which no promiscuous editing was observed at equivalent editing at C⁶⁶⁶⁶. A possibility for this could be the fortuitous introduction of a pair of tandem UGAU sequences within the intronic sequence 3′ of the editing site, a motif that has been previously shown to promote promiscuous editing (Sowden, M. P., et al. (1998) Nucleic Acids Res. 26:1644-1652.).

The description of the relationship of RNA splicing and editing is unique for apoB cytidine-to-uridine mRNA editing. However, an emerging theme in RNA processing is an interdependence of multiple steps in RNA maturation. Perhaps the most relevant to apoB editing is the adenine-to-inosine editing of glutamate and 5-hydroxytryptamine receptors. In contrast with apoB mRNA editing, mRNA substrates that undergo adenine-to-inosine editing all require the presence of a complementary intron sequence to form a partially double-stranded RNA structure that is recognized by the appropriate ADAR1or ADAR2 enzyme Simpson, L., et al. (1996) Annu. Re. Neurosci. 19:27-52; Maas, S., et al. (1997) Currr. Opin. Cell. Biol. 9:343-349; Rueter, S. M. and Emeson, R. B. (1998) Modification and Editing of RNA (Grosjean, H. and Benne, R., eds.), pp. 343-361.) . The critical role of cis-acting intronic sequences indicates deamination is a nuclear event, and as the editing site is frequently located close to a 5′ splice acceptor site (Higuchi, M., et al. (1993) Cell. 75:13.61-1370; Egebjerg, J., et al. (1994) Proc. Natl. Acad. Aci. U.S.A. 91:10270-10274.) suggests that the level of editing maybe influenced by interference or interaction with RNA splicing. For example, endogenously expressed GluR2 mRNA from neuronal cell lines is always edited to 100% at the Gln/Arg site, whereas unspliced GluR2 transcripts are edited to only 70-90% (Higuchi, M., et al. (1993) Cell. 75:1361-1370.), indicating a partial inhibition of splicing until editing has occurred. Conversely, the transcript of the Glu-R6 gene contains three exonic editing sites (Ile/Val, Tyr/Cys and Gln/Arg) which are edited to different extents, indicating that there must be a tightly regulated and coordinated action of the appropriate ADAR and the spliceosome at each editing site (Kohler, M., et al. (1993) Neuron 10:491-500; Seeburg, P. H., et al. (1998) Brain Res. Rev. 26:217-229.). In crosses of ADAR2± with GluR-B (R) +/+ mice, an influence from the editing status of the Gln/Arg site on subsequent splicing of the downstream intron was observed (Higuchi, M., et al. (2000) Nature 405:78-81.), indicating that these RNA processing events do not occur independently. The major steps in pre-mRNA processing, capping, splicing, 3′-end cleavage and polyadenylation are coupled to transcription through recruitment of the necessary processing factors to the largest subunit of the RNA polymerase II. This represents an efficient process for increasing local concentrations of related processing and transcription factors on pre-mRNAs as and when they are needed (Lewis, J. D., et al. (2000) Science 288:1385-1389.). Many analyses of RNA processing have attempted to identify active versus inactive populations of processing factors and have postulated that the greatest concentration of factors may or may not correspond to sites of function, dependent upon metabolic activity (Spector, D. (1993) Annu. Rev. Cell. Biol. 9:265-315.). Specifically, recent photobleaching studies (Lewis, J. D., et al. (2000) Science 288:1385-1389. and references cited therein) suggested that 'speckles′ correspond to sites where free small nuclear RNPs transiently assemble before recruitment by the C-terminal domain of RNA polymerase II and transfer to nascent transcripts. It is easily conceivable, therefore, that the processes of RNA editing and RNA splicing should be tightly coordinated, and the observation of nuclear and cytoplasmically localized APOBEC-1 and ACF corresponds to active and inactive complexes respectively. These two components of the minimal editosome, together with other editosomal proteins if necessary, could be rapidly recruited to newly synthesized apoB mRNA transcripts by a coordinated action of RNA polymerase II and spliceosome assembly.

Most, if not all, known RNA processing reactions can occur in vitro, but they are not as efficient as in vivo. This is also true for in vitro apoB RNA editing reactions. However, IVS-apoB RNA transcripts were edited with the same efficiency as intron-less apoB transcripts in vitro. This indicates that the presence of an intron per se does not interfere with editing, but, as was shown, there is a clear interdependence of splicing and editing for editing site regulation and fidelity in vivo. Such interdependence is also exhibited in mammalian nonsense-mediated decay (‘NMD’) of RNA, wherein only RNAs that contain nonsense codons and that have passed through the spliceosome are ‘marked’ and targeted for decay (Le Hir, H., et al. (2000) EMBO J. 19:6860-6869.). This imprinting of nuclear pre-mnRNA by proteins that remain bound in the cytoplasm is a means of mRNAs ‘communicating their history’ (Kataoka, N., et al. (2000) Mol. Cell. 6:673-682.) and/or perhaps ensuring that no further RNA processing/editing occurs in the cytoplasm (Maquat, L., et al. (2001) Ceel. 104:173-176.).

In conclusion, it has been demonstrated a spatial and temporal relationship between RNA splicing and apoB RNA editing. The suppression of editing-site utilization by proximal introns can explain the uniquely large size of exon 26 and/or the scarcity of other mooring-sequence-dependent cytidine-to-uridine editing sites. Moreover, these studies highlight the need to consider apoB RNA editing as an integrated process with RNA transcription and splicing, potentially expanding the number of auxiliary factors that should be considered as involved in apoB RNA editing.

### 4. Example 4

### a) Infectivity Assay using CEM15/Vif

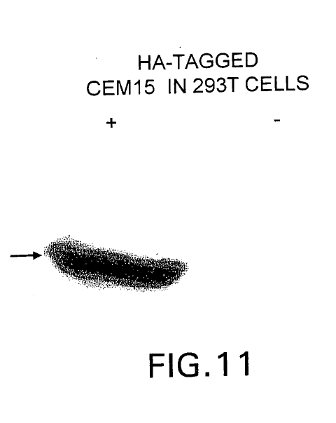

The infectivity assay was carried out in the context of Vif minus pseudotyped viruses and 293 T cells either lacking or expressing CEM15. An assay was developed using VSV G-protein pseudotyped lentiviral particles that confirmed the inhibitory effect of CEM15 on the infectivity of vif+ and vif− HIV-1 particles and is amenable to the rapid demarcation of the regions of HIV-1 DNA (or RNA) that is the target for CEM15 catalytic activity. An Env-deleted HIV-1 proviral DNA vector (derived from pNL43; AIDS Reagent Repository) was modified by replacement of Nef with a GFP reporter gene and two in-frame stop codons were inserted that abolished vif production (pHR-GFPΔVif) (confirmed by western blotting with anti-Vif antibodies (AIDs Reagent Repository). Stable, HA-tagged CEM15 expressing 293T cell lines were selected with puromycin and verified by western blotting with a HA specific monoclonal antibody (HA.11; BabCo) (FIG. 11). The expression of similar levels of full-length HA-tagged CEM15 (or mutant derivative thereof) can be assayed as well. Although structural modeling can predict focused mutations that impair deaminase activity without destabilizing the entire protein, expression of the mutants should be verified. The addition of the HA epitope tag has no effect on the ability of CEM15 to suppress infectivity (Sheehy et al. Nature 418:646-650, 2002). Isogenic HIV-1 pro-viral DNAs will be packaged into pseudotyped lentiviral particles by co-transfection with a plasmid encoding the VSV G-protein into 293T cells that lack endogenous CEM15 (−) or expressed wild type CEM15 (+) (FIG. 11). The resulting pseudotyped particles contain HIV-1 RNA of near full-length (with only a ˜2kb deletion) were quantified by reverse transcriptase (RT) assay. p24Gag protein content can also be assayed by ELISA to normalize viral particles. A defined number (1×10⁵ cpm of RT activity) of these particles were added to target, virus susceptible MT2 cells (5×10⁵). To assess their infectivity, the percentage of cells that expressed the GFP indicator gene encoded by the packaged recombinant HIV-1 genome was quantified 24 hours later by flow cytometry (University of Rochester Core Facility).

### b) Results

The results (FIG. 12) indicate that the expression of CEM15 in 293T cells resulted in at least a 100-fold decrease in Vif− viral infectivity compared to particles generated in parental 293T cells. The low level of GFP expression from vif−, CEM15+ particles is indistinguishable from background fluorescence in control cells [0.2%].

### 5. Example 5

### Vif Antagonist Peptides

The cellular deaminase CEM15 can introduce multiple and therefore catastrophic dC to dU mutations in negative strand viral DNA following reverse transcription. This anti-viral activity is due to the inherent catalytic activity of CEM15 on single stranded DNA and requires assembly of CEM15 within virions such that it is in position to interact with nascent cDNA during viral replication in the early stages of the HIV-1 life cycle. Antiviral activity of CEM15, however, can be blocked by the viral accessory protein known as viral infectivity factor or Vif (Sheehy et al. Nature 418: 646-650 (2002)).

Vif interacts with CEM15 and induces its poly-ubiquitination and degradation through the proteosome, thereby reducing the abundance of CEM15 and promoting viral infectivity. It has been discovered that Vif homodimers were required for Vif's interaction with CEM15 (Yang et al. J Biol Chem. 278(8): 6596-602 (2003), U.S. Pat. No. 6,653,443, herein incorporated by reference in their entirety).

All peptides described above that block Vif's interaction with CEM15 and/or act to prevent CEM15 polyubiquitination have the effect of maintaining CEM15 intracellular abundance in viral infected cells. The effectiveness in the peptide Vif antagonist to block Vif and thereby protect CEM15 from degradation is reflected as a sustained abundance of CEM15 and this can be monitored by western blotting whole cell extracts and probing these blots with anti-CEM15 antibodies that are biologically relevant and is a rapid assay for VDA activity, ultimately V peptide Vif antagonist activity. Changes in viral infectivity can be determined by ELISA quantification of HIV p24 antigen released from CEM15 positive cells that have been infected with wild type HIV-1 and treated with or without peptide Vif antagonists. Western blotting for CEM15 can be correlated with peptide Vif antagonist protection of CEM15 with VDA suppression of viral infectivity. These studies can be performed over a range of peptide Vif antagonist concentrations to establish a dose response relationship.

Commercially available services for high through put screening of chemical libraries can be used to identify small molecules that bind to the Vif dimerization domain peptide. These compounds can be tested for their ability to suppress CEM15 degradation and viral infected. CEM15, APOBEC-3F (h3F) and possibly APOBEC-3B (h3B), previously referred to as Phorbolins, (Jarmuz et al., Genomics, 79(3):285-96 (2002)) are co-expressed in human lymphoid and myeloid cells, and as is the case for APOBEC-1, can form homodimers and also heterodimers (Bogerd et al., Proc Natl Acad Sci USA 101(11):3770-4 (2004)). It has been shown that CEM15 and APOBEC-3F deaminate deoxycytidine on HIV-1 and HIV-2 minus strand cDNA. The dC to dU modifications template dG to dA mutations on the positive strand during replication, that inactivate multiple proteins essential for viral infectivity (Liu et al, J Virol, 78(4):2072-81 (2004). Zhang et al., Nature, 424(6944):94-8 (2003)). Unlike APOBEC-1 and other ARPs, CEM15, APOBEC-3F and APOBEC-3B establish a close proximity with viral genomes, by becoming integrated within virions during their assembly (Stopak et al., Mol Cell, 12:591-601 (2003); Gaddis et al., J. Virol, 77(10):5810-5820 (2003); Mariani et al., Cell. 114(1):21-31 (2003); Wiegand, et al., Embo J, 23(12):2451-8 (2004)). With regard to the deaminase activity, dimers of deaminases such as APOBEC-1 and AID are predicted to contain two catalytic centers (Xie et al., Proc Natl Acad Sci USA, 101(21): 8114-9 (2004)). From structural modeling, it appears that in the dimer, a flexible flap domain from one catalytic center interacts with the other catalytic center and thereby regulates nucleic acid substrate binding. CEM15, APOBEC-3F and APOBEC-3B monomers each have two catalytic centers (both of which have activity (Mangeat et al., Nature, 424(6944):99-103 (2003); Shindo et al., J Biol Chem, (2003)). Homo-and heterodimers of CEM15, APOBEC-3F and APOBEC-3B therefore are predicted to have four catalytic centers and are likely to have considerable combinatorial substrate targeting potential that provides the host cell with an adaptive advantage against a broad spectrum of viruses.

HIV-1 and HIV-2 use Vif to defeat the deaminase host defense. Vif has been shown to bind to both CEM15 and APOBEC-3F to target their ubiquitination and proteolytic degradation via the proteosome (Stopak et al. (2003); Mariani et al. (2003); Yu, X., et al. Science 302(5647):1056-60 (2003); Zheng et al. J Virol. 78(11):6073-6 (2004)). Vifs interaction with CEM15 occurs in a noncatalytic region that lies C-terminal to first catalytic domain. A single amino acid within this region (an aspartic acid in humans and a lysine in monkeys) provides the essential charge for the interaction of CEM15 with Vif (Bogerd et al. (2004); Mariani et al (2003), and Wiegand (2004)). Site-directed mutagenesis has shown that this single amino acid change in an ARP alters host range of a retroviruses (Bogerd et al. (2004), Mariani et al. (2003) and Xu et al., Proc Natl Acad Sci USA, 101(15):5652-7 (2004). Due to this single amino acid difference simian virus (SIV) derived Vif cannot bind to human CEM15 and vise versa and consequently there is species-specific exclusion of CEM15 from the virion. Consequently, this region of CEM15 and APOBEC-3F can constrain the extent to which Vif can mutate and still protect the virus from the ARP-based host defense.

Vif forms homodimers, and Vif dimerization is required for viral infectivity. It has also been shown that Vif dimerization is required for Vif-dependent destruction of CEM15. Therefore, the Vif dimerization domain is a drug target for suppressing viral infectivity. HIV is notorious for its hypermutability and the acquired resistance of this virus to therapy in AIDS patients. Vif has to interact with host cell CEM15 to protect the virus and therefore loss of Vif dimerization capacity through mutation may be less tolerated than are mutations in other viral proteins that have enabled the virus to acquire resistance to current therapeutic approaches.

### a) Experimental

CEM15 abundance can be quantified by western blotting as described above. Small molecules that bind to any of the aforementioned peptides can be evaluated for their ability to protect or restore CEM15 abundance using the aforementioned western blotting systems of whole cell extracts of cells that have been transfected with CEM15 and Vif wherein these proteins are co-expressed in 293T cells (conditions that result in CEM15 destruction) and evaluated (by western blotting of cell extracts) for the ability of VDA peptides of varying size and sequence to restore CEM15 abundance. Co-expression of CEM15 and Vif by transfection in 293T cells results ˜99% ablation of intracellular CEM15 within 36-48 h post-transfection. Transduction of VDA into cells 6-12 hours following transfection results in restoration of CEM15. All peptides are tested according to this schedule. Expression of CEM15 and Vif are driven from the CMV promoter of pcDNA3 plasmids.

Determination of changes in endogenous CEM15 abundance. H9 cells express sufficient CEM15 that it is readily detectable by western blotting cell extracts with monoclonal anti-CEM15 antibodies (4F11/H1A, AIDS Research and Reference Reagent Program). This affords the opportunity to correlate viral infectivity measurements with endogenous CEM15 levels as the efficacy of optimized peptide Vif antagonists are evaluated in protecting endogenous CEM15 from Vif-dependent degradation. All assays of viral infectivity and the quantification of CEM15 are performed in triplicate. Cells can be lysed and extracts blotted and reacted with antibodies as described above using the signal from GAPDH as a normalization value for comparing CEM15 levels between treatment groups.

Small molecules that bind to the Vif dimerization domain and evaluate their ability to block Vif dimerization, prevent CEM15 degradation and suppression HIV-1 infectivity. Peptides corresponding to the Vif dimerization can be used to screen chemical libraries for interacting compounds.

Analysis of the initial hits. The screen can yield numerous compounds. Although the number of ‘hits’ can be greater had using full length Vif in the screening assay, probing the libraries with peptides containing Vif's dimerization domain selects for interactions that are more relevant to that domain and therefore compounds that are selected in this way stand the greatest possibility of having antiviral activity through that mechanism.

Once interacting compounds have been identified, the initial evaluations can be done based on their ability to restore CEM15 abundance in Vif expressing cells using the western blotting assay described previously. This assay was chosen for the initial analysis of compounds over infectivity assays because given that CEM15 stability is widely accepted as a reliable predictor of viral infectivity, it is more rapid, cheaper and has a significantly lower biohazard risk. The screening narrows the pool of selected candidates from the initial screen to a half dozen or less compounds (SMVA candidates) for further validation. A dose range and time course in which maximum restoration of CEM15 abundance can occur can also be established.

These SMVA candidates then move on to secondary biological end point evaluations. This involves analysis of their ability to supress live virus infectivity as described above. Dose response curves can be established for all compounds that block viral infectivity.

Wash conditions varying in ionic strength, pH, detergent concentration, chaotropic agents or competitors are employed as a means to reduce nonspecific interaction and enrich for interactions with the highest specificity (lowest Kd).

### 6. Example 6

### Reverse Transcription and Packaging Independent Antiviral Activity of CEM15

### a) Summary

CEM15 (a.k.a. APOBEC-3G or h3G) functions as a natural defense against HIV-1 viral infectivity by mutating the viral genome during its reverse transcription. This activity is inhibited by HIV-1 viral infectivity factor (Vif) that is able to trigger degradation of CEM15 and prevent it from being packaged into the virion. However, this antiviral protein appears to have additional means by which it suppresses HIV-1.

Cells were transfected with provirus DNA that produce pseudotyped viral particles in the absence of reverse transcription. CEM15 expression induced a marked (100-fold) reduction in viral particle production in the absence of Vif compared to that obtained from control cells or in the presence of Vif. This effect was due to a selective and marked reduction in viral protein and RNA. Reduction in viral particle production was also observed with a catalytically inactive mutant of CEM15 showing that deaminase activity was not responsible for this antiviral mechanism. Vif expression blocked the effect of both CEM15 and the catalytic mutant CEM15 on viral production by inducing their degradation.

It was demonstrated that recombinant CEM15 can bind directly to RNA, which shows that it can play a role in the reduction of viral RNA. The phenotype described here differs from that in other reports in that it does not require CEM15 to become incorporated within virions or have mutagenic activity during reverse transcription. This mechanism can contribute important antiviral activity during late stages of the viral life cycle.

### b) Introduction

Reverse transcription-dependent mutational activity of CEM15 on HIV-1 ssDNA is not the only means by which CEM15 can reduce viral infectivity. In fact, mutations in one or both of the zinc-dependent cytidine deaminase domains did not ablate CEM15's antiviral activity (Shindo et al., J Biol Chem (2003)). Moreover, blockage of reverse transcriptase (RT) processivity by CEM15 binding to the viral RNA templates has been suggested as an additional antiviral mechanism (Li et al., J Cell Biochem 92, 560-572 (2004)). In support of multiple mechanisms, transient expression of CEM15 reduced the level of pseudotyped HIV-1 particles generated from producer cells that were co-transfected with replication-defective proviral DNA constructs and helper plasmids (Sheehy et al., Nature 418, 646-650 (2002)). This antiviral activity would have had to involve a mechanism that was independent of reverse transcription.

It is shown that stably expressed CEM15 significantly reduced the level of pseudotyped HIV-1 (particles lacking Vif. The reduced viral particle production is the result of a selective suppression of viral RNA leading to reduction in essential HIV-1 proteins. These effects were not observed when Vif was expressed due to the marked reduction of CEM15. Although CEM15 was required to deplete viral particle production its deaminase function was not necessary. The data indicate an antiviral mechanism in producer cells which is potentially significant late during the viral life cycle that involves directly or indirectly the RNA binding ability of CEM15 and does not require virion incorporation of CEM15 nor viral replication.

### c) Experimental Procedures

Plasmid Constructions. CEM15 cDNA was RT-PCR amplified from oligo(dT)-primed total cellular RNA from CEM. cells (Sheehy et al (2002). CEM15 deaminase domain mutations (DM) [E67A, E259A] were created by site-directed mutagenesis using the Quikchange system (Stratagene). Wild type CEM15 and DM were subcloned with an amino-terminal 6×His and HA (hemagglutinin) tag into pIRES-P to permit CMV promoter driven expression of the cDNA and puromycin selection from an ECMV IRES element. pDHIV-GFP (from Dr. V. Planelles) is a pNL4-3 derived HIV-1 vector that contains a deletion of the env gene. pDHIV-GFP/ΔVif was constructed by inserting a 12 bp fragment (5′-TAGTAACCCGGG-3′, SEQ ID NO: 62) containing two termination codons underlined) at the PflM1 site of pDHIV-GFP that lies near residue 89 of Vif, thereby leading to the production of a truncated and nonfunctional vif gene product. Cell culture and Transfection-293T cells obtained from ATCC (Manassas, Va.) were maintained in DMEM. containing 10% fetal bovine serum plus penicillin/streptomycin/fungizone (Cellgro), and Non-Essential Amino Acids (Invitrogen) and were transfected using FuGENE 6 (Roche Molecular Biochemicals). Clonal cell lines were obtained by limiting dilution under 1 μg/ml puromycin selection.

Virus production. A two plasmid system was used to generate pseudotyped HIV-1 particles. 293T cells stably expressing CEM15, DM, or empty pIRES-P vector were transfected with a mixture of pVSV-G and pDHIV-GFP (wt Vif) or pDHIV-GFP/ΔVif using Lipofectamine 2000 (Invitrogen). Viruses were harvested at 48 and 72 hour post-transfection from culture supernatants and concentrated by ultracentrifugation (22 K rpm, 2 hour at 4° C.).

p24 and viral infectivity assays. Serial dilutions of viral stocks were assayed for p24 according to the manufacturer's recommendations (Beckman-Coulter, F L) and only results within the linear range of the standard curve were considered. Serially diluted viral stocks, normalized base on p24, were used to infect HeLa cells. 48 hours post-infection, cells were fixed and GFP expression analyzed by microscopy and flow cytometric analysis.

Cell lysates and western blot analysis. Cells were harvested by scraping into PBS containing a cocktail of protease inhibitors (0.5 μg/mL each of aprotinin, pepstatin, and leupeptin, 1 mM. PMSF (USB Corp), 2 mM. Benzamidine and 2 mM EGTA) at 24, 48, and 72 hours following transient transfection with HIV-1 plasmids. Cell pellets were lysed in Reporter lysis buffer (Promega) containing protease inhibitor cocktail. Protein concentrations were determined using the Bradford Assay (BioRad), and equivalent amounts of protein were analyzed by SDS-PAGE and subsequent western blotting using antibodies specific for HA (tagged CEM15 and DM), □-actin, and HIV-1 RT (#6195), p24 (#287), Vif (#6459), Tat (#705) and Vpr (#3951) (Hauber et al., Proc Natl Acad Sci USA 84, 6364-6368 (1987); Simon et al., J Virol 71:5259-5267 (1997), Simon et al. J Virol 69:4166-4172 (1995), Fouchier et al. J Virol 70:8263-8269 (1996)). Protein-RNA crosslinking. The indicated amounts of recombinant CEM15 (#10068, ImmunoDiagnostics, Inc., AIDS Reagent Repository) were added to 50 μl binding reactions containing 10 mM. Hepes pH 7.9, 10% glycerol (v/v), 50 mM KCl, 50 mM. EDTA, 0.25 mM. DTT, 40 units of RNasin®b (Promega), and 20 fmols of gel purified GP-RNA (nt 1573-2261; accession #K02013) or apoB RNA (nt 6413-6860) (Smith, H. D., Methods 15:27-39 (1998)) that was ³²P[ATP and CTP] labeled during in vitro T7 polymerase transcription (Promega). RNA binding reactions were incubated at 30° C. for 3 h as previously described (Smith (1998)). Reactions were exposed to short wavelength ultraviolet (UV) light to induce protein-RNA crosslinking and subsequently digested with RNase A and T1 as previously described (Smith (1998)). Northern blot analysis. PolyA+RNA prepared with a MicroPoly(A) Purist Kit (Ambion) according to manufacturer's protocol was resolved on a formaldehyde agarose gel and transferred to nylon. The probe was GP-RNA cDNA radiolabeled with ³²P[dCTP] using Ready-To-Go DNA labeling beads (Amersham Biosciences) according to the manufacturer's protocol. Blots were hybridized to the probe (1×10⁶ cpm/ml) in ExpressHyb (Clontech) and washed according to the manufacturer's recommendations.

Blots were then stripped and reprobed with adenovirus EIA cDNA radiolabeled with ³²P[dCTP] as stated above.

### d) Results

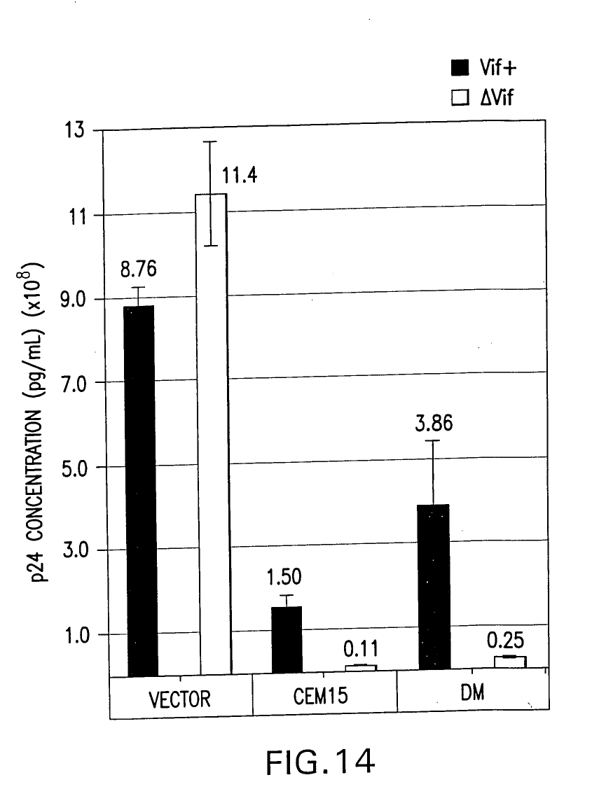

To investigate alternative mechanisms that may contribute to the antiviral activity of CEM15, 293T cell lines stably expressing CEM15 (293T-CEM15) were selected and transfected with plasmids containing replication-defective (Env-deleted) HIV-1 proviruses (Vif+ or ΔVif) plus a helper/packaging plasmid (encoding VSV-G). Culture supernatants from these cells were then assayed by p24 ELISA, and a marked reduction of viral particle production (100-fold) by the ΔVif construct was detected in 293T-CEM15 versus the control, a 293T stable cell line containing pIRES-P vector (FIG. 14). In contrast, Vif+ provirus culture supernatants contained abundant viral particles, only 5-fold below control cells (FIG. 14). The infectivity of the pseudotyped virus preparations was examined by transduction of HeLa cells with p24-normalized amounts of Vif+ and ΔVif virus particles. Consistent with prior reports (Shindo et al. (2003), Liu et al., J Virol 78:2072-2081 (2004), Mangeat et al. Nature 424:99-103 (2003)), the infectivity of ΔVif pseudotyped HIV-1 particles was markedly reduced compared to the Vif+ viruses. The data demonstrated ΔVif viral particle production could be significantly suppressed by CEM15 expression, and that this effect could be overcome by Vif expression. Moreover the data indicated that there were two general effects of CEM15 expression; one that manifests in reporter cells due to CEM15 incorporation with virions and mutagenic deaminase activity during reverse transcription and a previously uncharacterized effect on viral particle production in producer cells.

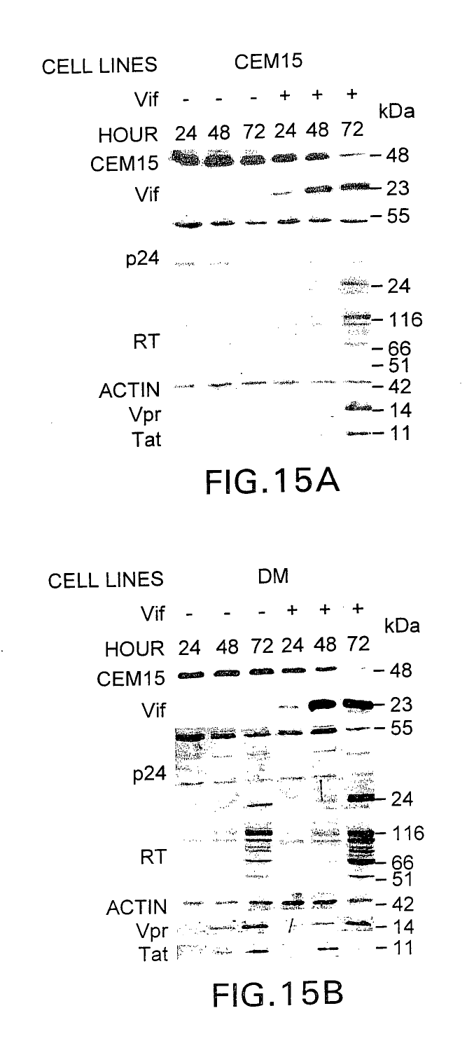

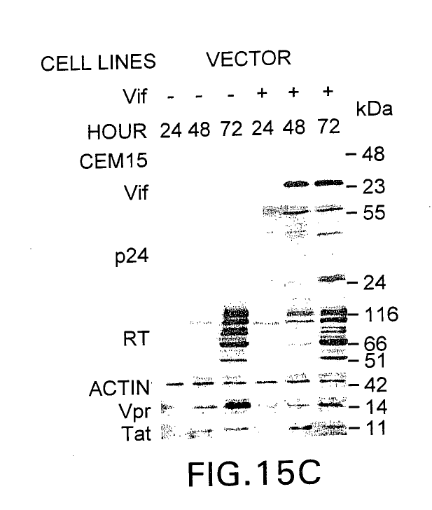

To evaluate whether the suppression of viral production was due to reduced viral protein abundance, following transfection with HIV-1 proviral plasmid DNAs, cell lysates were prepared from an equivalent number of cells, normalized for the amount of protein, and evaluated by western blotting. CEM15 was expressed at similar levels throughout the 72 hour period, however, the abundance of CEM15 was markedly decreased over the same time period in cells expressing functional Vif (FIG. 15A). These findings are consistent with the ability of Vif to target CEM15 for proteolysis (Mariani et al. Cell 114:21-31 (2003), Stopak et al. Mol Cell 12:591-601 (2003), Yu et al. Science 302:1056-1060 (2003)), but they also indicate that the level of CEM15 expression in our 293T stable cell lines is within a range that can be functionally suppressed by proviral Vif expression.

Consistent with the reduction in viral particle production (FIG. 14), a marked reduction in HIV-1 p24 and RT protein was observed in 293T-CEM15 cells transfected with the Vif proviral DNA plasmids (compare FIGS. 15A and C at 72 h). In contrast, 293T-CEM15 transfected with Vif+ proviral DNA plasmid contained comparatively elevated levels of p24 and RT (FIG. 15A at 72 h). Similar effects were also observed for the HIV-1 regulatory protein, Tat, and the accessory protein, Vpr (FIGS. 15A and C). These reductions in viral proteins were selective, since β-actin levels in the various lysates were virtually identical at all time points (FIGS. 15A-C; note that lane-loading was normalized on the basis of total protein amount loaded). Furthermore, luciferase expression from a co-transfected plasmid was also unaffected by CEM15 expression confirming that CEM15-mediated repression has viral specificity.

CEM15 is predicted to contain two zinc-dependent deaminase domains (Wedekind et al. Trends Genet 19:207-216 (2003)), each of which has been shown to possess partial antiviral activity (Shindo et al. (2003)). Point mutations of the essential glutamate residue within each catalytic domain reduced significantly, but did not abolish CEM15-mediated inhibition of HIV-1 infectivity (Mangeat et al. (2003)). To evaluate whether deaminase activity was required for the observed suppression of viral particle production, a 293T cell line stably exp-ressing-the CEM15 double mutant E67A/E259A (DM) was transfected with Vif+ or □Vif proviral DNA plasmids. As shown in FIG. 14, expression of DM. resulted in a strong inhibition of HIV-1 particle production in the absence of Vif (approx. 50-fold, compared to control cells). This suppression was roughly 2 to 2.5 fold weaker than that produced by wild-type CEM15, and could be overcome by expression of Vif (Vif+ virus). Consistent with this, expression of DM. also reduced the levels of p24 and RT in the absence of Vif, although not to the same level as in 293T-CEM15 cells (compare FIGS. 15A and B). Effects on Tat and Vpr were somewhat more variable. These data suggested that a functional deaminase domain is important for the reduction in HV-1 particle production, but is not a requirement. In considering how CEM15 might alter viral protein production, the possibility was evaluated that it might be acting on proviral plasmid DNA or viral RNA in the nucleus. Previous immunocytochemical analysis of HA-tagged CEM15 in 293T cells suggested a predominant if not exclusive cytoplasmic localization (Mangeat et al. (2003)). However, this observation does not preclude the possibility that, like homologous proteins such as AID and APOBEC-1, CEM15 can shuttle between the nucleus and cytoplasm (Chester et al. Embo J 22:3971-3982 (2003), Yang et al., Exp Cell Res 267:153-164 (2001), Ito et al. Proc Natl Acad Sci USA 101:1975-1980 (2004)). This kind of trafficking could permit CEM15 to act on double-stranded proviral plasmid DNA or on ss-plasmid DNA (during transcription), leading to mutation and/or degradation of proviral template. This possibility was evaluated on proviral DNA isolated from 293T-CEM15 cells and control cells. No difference in DNA recovery was detected in 293T-CEM15 transfected with ΔVif provirus compared to control cells transfected with +Vif provirus, and no dC to dU mutations in proviral DNA were evident as determined by uracil DNA glycosylase treatment of isolated viral DNA and alkaline cleavage of a pyrimidinic sites (Suspene et al. Nucleic Acids Res 32:2421-2429 (2004)). Thus, it was concluded that DNA mutational activity by CEM15 in producer cells did not account for the reduced viral particle production.

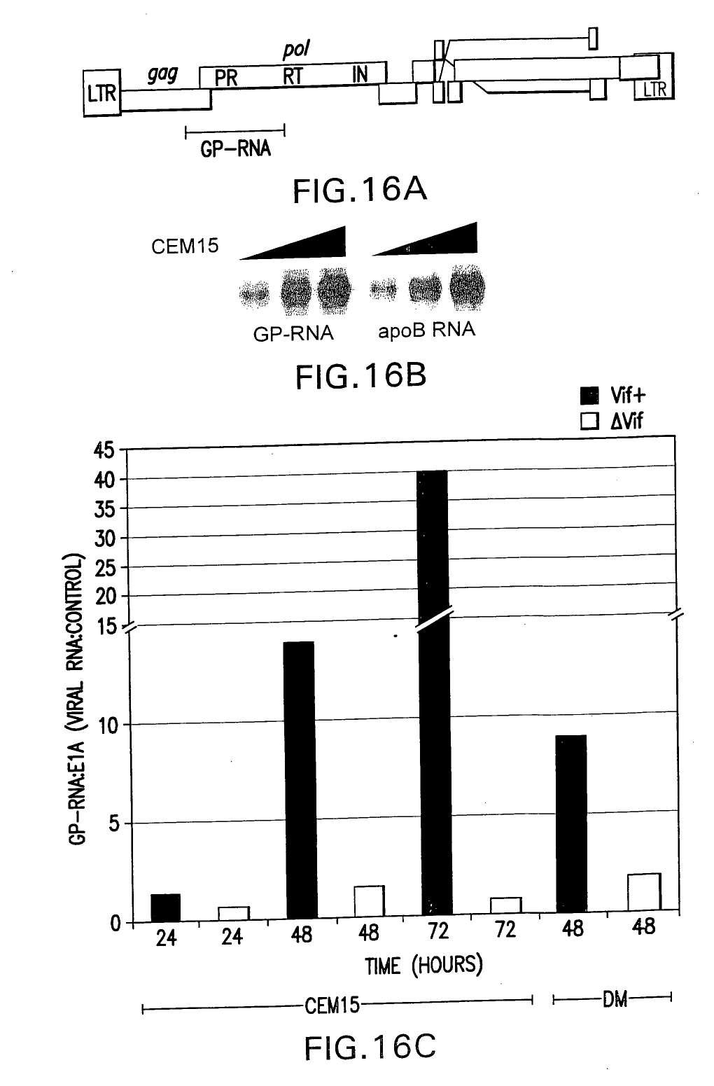

It was also examined whether CEM15 might have the ability to selectively target the frameshift region in the viral Gag-Pol mRNA. This was of interest in part because of the effect of CEM15 on the stability and proteolytic processing of the Gag precursor (FIG. 15A), and also because of the Gag-Pol junction stem-loop structure that is necessary for the minus one frameshift translation of Gag-Pol (Baril et al. Rna 9:1246-1253 (2003), Frankel et al. Annu Rev Biochem 67:1-25 (1998)). CEM15-dependent RNA editing activity or RNA binding activity as reported for APOBEC-1 (MacGinnitie et al. 270:14768-14775 (1995), Anant et al. Mol Cell Biol 20:1982-1992 (2000)) could disrupt secondary structure or otherwise mutate coding capacity. To test for RNA editing, polyA+ RNA from 293T-CEM15 72 h post-transfection of ΔVif or Vif+ proviral plasmid DNAs was RT PCR amplified with primers for the Gag-Pol junction and protease region (GP-RNA, FIG. 16A). 12 and 8 clones from ΔVif or Vif+ conditions (respectively) were sequenced and all were found to be identical to the original HIV-1 DNA, eliminating RNA editing ofthis region as a mechanism.

CEM15 RNA binding capacity was determined in-vitro using purified recombinant CEM15 and radiolabeled RNA in our standardized ultraviolet light (UV) crosslinking assay (Smith, H. D. (1998), Galloway et al. 34: 24-526, 528, 530 (2003)). CEM15 bound to radiolabeled HIV-1 GP-RNA in concentration dependent manner (FIG. 16B) however the yield of complexes was similar with an equivalent amount and specific activity of radiolabeled apoB mRNA containing the RNA editing site for APOBEC-1 (MacGinnitie et al. (1995) and Snant et al. (2000)). The nonselective interaction of CEM15 with RNA is consistent with reports suggesting that RNA binding activity of CEM15 blocks RT progression on viral RNA (Li et al. (2004)) and that its interactions with viral and cellular RNAs enable CEM15 to assemble with virions (Svarovskaia et al. J Biol Chem (2004)). However, through the use of recombinant protein, it was established that CEM15 can bind to RNA in the absence of additional protein factors.

Considering the ability of CEM15 to interact with RNA, it was tested whether it could modify the stability of the viral Gag-Pol mRNA. To evaluate mRNA stability, polyA+ RNA was collected from 293T-CEM15 at 24 h, 48 h and 72 h after transfection with proviral DNAs, and northern blot analysis was performed. The results revealed that viral RNA levels were depleted at all time points, in the absence of Vif (2-fold, 9-fold and 56-fold respectively, when compared to cell transfected with the Vif+ provirus) (FIG. 16C). CEM15 expression did not affect the abundance of an endogenous transcript present in 293T cells (adenovirus E1A RNA), as expected since luciferase and β-actin protein expression were also unaffected by CEM15. Hence E1A RNA served as an internal loading control for comparison of viral RNA levels (FIG. 16C). Expression of the deaminase inactive DM also induced a depletion of viral RNA but to a lesser extent (consistent with the recovery of viral proteins; FIGS. 15B and 16C). Taken together with the aforementioned studies, these findings show that CEM15 binding to viral RNA alone or in conjunction with other viral or cellular proteins may have signaled for viral RNA degradation.

### e) Discussion

A considerable body of evidence indicates that the suppression of HIV-1 infectivity by CEM15 is due to a pleiotropic effect arising from its ssDNA mutating cytidine deaminase activity during viral RNA genome reverse transcription (Yu et al. Nat Struct Mol Biol 11:435-442 (2004); Harris et al. Cell 113:803-809 (2003); Zhang et al. Nature 424, 94-98 (2003)). Studies in which either or both of the cytidine deaminase domains of CEM15 were mutated showed that both catalytic domains are fuinctional in mutating HIV-1 minus strand cDNA genomes (Shindo et al. (2003)). However, these studies also demonstrated partial suppression of viral infectivity by deaminase inactive CEM15. A role for CEM15 that does not involve ssDNA mutation has been suggested at the level of blocking the progression of reverse transcription on viral RNA templates (Li et al. (2004)). A novel mechanism was evaluated whereby CEM15 suppressed HIV-1 production, which does not depend on the incorporation of CEM15 into the virion and/or viral reverse transcription. It was shown that CEM15 selectively reduced viral RNA and protein abundance resulting in a phenotype of reduced viral particle assembly. This effect was not dependent upon CEM15-mediated DNA mutation or RNA editing and was largely abrogated by the expression of Vif. It was also revealed that recombinant CEM15 can bind directly to viral Gag-Pol RNA and non-viral RNAs. These findings corroborate recent reports of CEM15's general RNA binding activity (Yu et al. (2004), Li et al. (2004), Svarovskaia et al. (2004)) and indicate that, either directly or indirectly, CEM15 binding to viral RNA can lead to its premature decay. In this regard, CEM15 interactions with Gag nucleocapsid (Cen et al. J Biol Chem (2004), Alce et al. J Biol Chem (2004)), and the ability of both proteins to bind HIV-1 RNA can provide specificity resulting in the selective degradation of viral RNAs.

Previously, a significant impairment in viral production by CEM15-expressing 293T cells (Lin et al. (2004), Kao et al. J Virol 77:11398-11407(2003)) has not been shown, but these experiments either relied upon a transient transfection of CEM15 (raising the possibility that some cells may have received HIV-1 DNA in the absence of CEM15) (Kao et al. J Virol 77:11398-11407(2003)) or they have involved stable co-expression of both CEM15 and proviral DNA (Lin et al. (2004)). In the latter case, drug selection was used to establish the stable cell clones; which may have resulted in a powerful, positive selective pressure for rare cell clones in which hygromycin resistance gene (which was driven by the HIV-1 LTR, and inserted into the pol region of the genome) was highly expressed. The ability to uncover the effect of CEM15 on viral RNA stability and protein production is therefore attributed to the fact that stable cell clones were used that uniformly express CEM15.

It was of interest that CEM15 expression had a differential effect on viral protein abundance. The expression of the 55 kDa Gag precursor (p55) in proviral transfected 293T-CEM15 cells was similar, regardless of whether Vif was expressed, but p24 abundance was markedly reduced in the absence of Vif (FIG. 15A). The elevated levels of the p55 in 293T-CEM15 cells and DM. cells transfected with □Vif provirus, compared to control cells and DM. cells transfected with +Vif provirus (where p55 undergoes rapid and efficient cleavage) throughout the 72 hours suggested a lack of protease activity (compare FIG. 15A and B −Vif, contrast to B +Vif and C). Furthermore, products of protease cleavage reactive with the RT-specific antibody were undetectable in 293T-CEM15 cells in the absence of functional Vif (FIG. 15A) (proteins detected included the product of initial protease cleavage [˜116 kDa] (de Oliveira et al. J Virol 77:9422-9430 (2003)) and the fully processed RT heterodimer [p66 and p51] (Frankel et al. (1998), de Oliveira et al. (2003)). Collectively, these results suggested that functional protease activity in CEM15 expressing cells was greatly diminished, possibly due to low amounts or the absence of the Gag-Pol precursor.

In conclusion, it appears that CEM15 can exert an antiviral effect during both the early and late phases of the HIV-1 life cycle.

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### I. REFERENCES

Alberts, B., D. Bray, J. Lewis, M. Raff, K. Roberts and J. D. Watson Molecular Biolom of the Cell. (3rd ed.) Garland Pub. Inc. NY, N.Y. (1994).
Anant, S. and N. O. Davidson, Molecular mechanisms of apolipoprotein B mRNA editing. i Curr Opin Lipidol. 12(2):159-65 (2001).
Anant, S. G., Giannoni, F., Antic, D., DeMaria, C. T., Keene, J. D., Brewer, G. and Davidson, N. O. AU-rich RNA binding proteins Hel-N1 and AUF1 bind apolipoprotein B mRNA and inhibit posttranscriptional C to U editing. Nucleic Acids Symp. Ser. 36, 115-118 (1997).
Anant, S., et al., ARCD-1, an apobec-1-related cytidine deaminase, exerts a dominant negative effect on C to U RNA editing. Am J Physiol Cell Physiol. 281:C1904-16 (2001).
Anant, S., et al., Evolutionary origins of the mammalian apolipoproteinB RNA editing enzyme, apobec-1: structural homology inferred from analysis of a cloned chicken small intestinal cytidine deaminase. Biol Chem. 379:1075-81 (1998).
Anant, S., MacGinnitie, A. J. and Davidson, N. O. APOBEC-1, the catalytic subunit of the mammalian apoB B mRNA editing enzyme, is a novel RNA-binding protein. J. Biol. Chem. 270, 14762-14767 (1995).
Andersson, T., C. Furebring, C. A. Borrebaeckand S. Pettersson, Temporal expression of a V(H) promoter-Cmu transgene linked to the IgH HS 1,2 enhancer. Mol Immunol, 36(1):19-29 (1999).
Arakawa, H., J. Hauschildand J. M. Buerstedde, Requirement of the activation-induced deaminase (AID) gene for immunoglobulin gene conversion. Science, 295(5558): p.1301-6 (2002).
Arulampalam, V., C. Furebring, A. Samuelsson, U. Lendahl, C. Borrebaeck, I. Lundkvistand S. Pettersson, Elevated expression levels of an Ig transgene in mice links the IgH 3′ enhancer to the regulation of IgH expression. Int Immunol. 8(7):1149-57 (1996). Backus, J. W. and Smith, H. C. Apolipoprotein B mRNA sequences 3′ of the editing site are necessary and sufficient for editing and editosome assembly. Nucleic Acids Res. 19(24):6781-6786 (1991).
Backus, J. W. and Smith, H. C. Specific 3′ sequences flanking a minimal apoB mRNA editing ‘cassette’ are critical for efficient editing in vitro. Biochim. Biophys. Acta 1217, 65-73 (1994).
Backus, J. W. and Smith, H. C. Three distinct RNA sequence elements are required for efficient apoB RNA editing in vitro. Nucleic Acids Res. 22, 6007-6014 (1992). Backus, J. W., Schock, D. and Smith, H. C. Only cytidines 5′ of the apoB niRNA mooring sequence are edited. Biochim. Biophys. Acta 1219(1):1-14 (1994).
Barat, C., V. Lullien, O. Schatz, G. Keith, M. T. Nugeyre, F. Gruninger-Leitch, F. Barre-Sinoussi, S. F. LeGrice, and J. L. Darlix, HIV-1 reverse transcriptase specifically interacts with the anticodon domain of its cognate primer tRNA. Embo J. 8(11):3279-85 (1989).
Baum, C. L., Teng, B. B. and Davidson, N. O. Apolipoprotein B messenger RNA editing in the rat liver: modulation by fasting and refeeding a high carbohydrate diet. J. Biol. Chem. 265, 19263-19270 (1990).
Berkhout, B., A. T. Das, and N. Beerens, HIV-1 RNA editing, hypermutation, and error-prone reverse transcription. Science 292(5514):7 (2001).
Bernstein, E., A. M. Denliand G. J. Hannon, The rest is silence. RNA 7(11):1509-21 (2001).
Betts L., Xiang S, Short S A, Wolfenden R, Carter C W Cytidine deaminase. The 2.3 A crystal structure of an enzyme: transition-state analog complex. J Mol Biol. 235, 635-56 (1994).
Blanc, V., et al. Mutagenesis of apobec-1 complementation factor reveals distinct domains that modulate RNA binding, protein-protein interaction with apobec-1, and complementation of C to U RNA-editing activity. J Biol Chem. 276(49):46386-93 (2001).
Blanc. V., Navaratnam, N., Henderson, J. O., Anant, S., Kennedy, S., Jarmuz, A., Scott, J. and Davidson, N. O. Identification of GRY-RBP as an apo B mRNA binding protein that interacts with both apobec-1 and with apobec-1 complementation factor (ACF) to modulate C to U editing. J. Biol. Chem. 276(13):10272-10283 (2001).
Bostrom, K., Garcia, Z., Poksay, K. S., Johnson, D. F., Lusis, A. J. and Innerarity, T. L. Apolipoprotein B mRNA editing. Direct determination of the edited base and occurrence in non-apolipoprotein B producing cell lines. J. Biol. Chem. 265, 22446-22452 (1990).
Bouhamdan, M., S. Benichou, F. Rey, J. M. Navarro, I. Agostini, B. Spire, J. Camonis, G. Slupphaug, R. Vigne, R. Benarous, and J. Sire, Human immunodeficiency virus type 1 Vpr protein binds to the uracil DNA glycosylase DNA repair enzyme. J Virol. 70(2):697-704 (1996).
Bourara, K., S. Litvak, and A. Araya, Generation of G-to-A and C-to-U changes in HIV-1 transcripts by RNA editing. Science. 289(5484):1564-6 (2000).
Bowie, J. U., R. Luthy, and D. Eisenberg, A method to identify protein sequences that fold into a known three-dimensional structure. Science. 253(5016):164-70 (1991).
Bransteitter, R., P. Pham, M. D. Scharff, and M. F. Goodman, Activation-induced cytidine deaminase deaminates deoxycytidine on single-stranded DNA but requires the action of RNase. Proc Natl Acad Sci USA. 100(7): p. 4102-7 (2003).
Bross, L., M. Muramatsu, K. Kinoshita, T. Honjoand H. Jacobs, DNA Double-Strand Breaks: Prior to but not Sufficient in Targeting Hypermutation. J Exp Med 195(9):1187-1192 (2002).
Burley, S. K. An overview of structural genomics. Nature Struct. Biol. 7, 932-934 (2000).
Camaur, D. and D. Trono, Characterization of human immunodeficiency virus type 1 Vif particle Liu, H., X. Wu, M. Newman, G. M. Shaw, B. H. Hahn, and J. C. Kappes, The Vif protein of human and simian immunodeficiency viruses is packaged into virions and associates with viral core structures. J Virol, 69(12): p. 7630-8.incorporation. J Virol. 70(9):6106-11 (1996).
Carlow, D. C., A. A. Smith, C. C. Yang, S. A. Short, and R. Wolfenden, Major contribution of a carboxymethyl group to transition-state stabilization by cytidine deaminase: mutation and rescue. Biochemistry. 34(13):4220-4 (1995).
Cartegni, L., S. L. Chewand A. R. Krainer, Listening to silence and understanding nonsense: exonic mutations that affect splicing. Nat Rev Genet. 3(4):285-98 (2002).
Casellas, R., A. Nussenzweig, R. Wuerffel, R. Pelanda, A. Reichlin, H. Suh, X. F. Qin, E. Besmer, A. Kenter, K. Rajewsky and M. C. Nussenzweig, Ku80 is required for immunoglobulin isotype switching. Embo J 17(8):2404-11 (1998).
Cattaneo, R. Biased (A→I) hypermutation of animal RNA virus genomes. Curr Opin Genet Dev 4(6): 895-900 (1994).
Chaudhuri, J., M. Tian, C. Khuong, K. Chua, E. Pinaud, and F. W. Alt, Transcription-targeted DNA deamination by the AID antibody diversification enzyme. Nature. 422(6933):726-30 (2003).
Chen, J., R. Lansford, V. Stewart, F. Youngand F. W. Alt, RAG-2-deficient blastocyst complementation: an assay of gene function in lymphocyte development. Proc Natl Acad Sci USA. 90(10): 4528-32 (1993).
Chen, R., H. Wang, and L. M. Mansky, Roles of uracil-DNA glycosylase and dUTPase in virus replication. J Gen Virol. 83(Pt 10):2339-45 (2002).
Chen, S. H., Habib, G., Yang, C. Y., Gu, Z. W., Lee, B R., Weng, S. A., Silberman, S. R., Cai, S. J., Deslypere, J. P., Rosseneu, M., Gotto, A. M. J. R., Li, W. H. and Chan, L. Apolipoprotein B-48 is the product of a messenger RNA with an organ-specific in-frame stop codon. Science 238, 363-366 (1987).
Chothia, C. and A. M. Lesk, The relation between the divergence of sequence and structure in proteins. Embo J. 5(4):823-6 (1986).
Chua, K. F., F. W. Altand J. P. Manis, The Function of AID in Somatic Mutation and Class Switch Recombination: Upstream or Downstream of DNA Breaks. J Exp Med. 195(9): F37-41 (2002).
Courcoul, M., C. Patience, F. Rey, D. Blanc, A. Harmache, J. Sire, R. Vigne, and B. Spire, Peripheral blood mononuclear cells produce normal amounts of defective Vif-human immunodeficiency virus type 1 particles which are restricted for the preretrotranscription steps. J Virol. 69(4):2068-74 (1995).
Dance, G. S. C., Sowden, M. P., Yang, Y. and Smith, H. C. APOBEC-1 dependent cytidine to uridine editing of apolipoprotein B RNA in yeast. Nucleic Acids Res. 28, 424-429 (2000).
Dance, G. S. C., Beemiller, P., Yang, Y., Van Mater, D. Mian, S. I. and Smith, H. C. Identification of the yeast cytidine deaminase CDD1 as an orphan C to U RNA editase. Nucleic Acids Res. 29, 1772-1780 (2001).
Dance, G. S. C., Sowden, M. P., Cartegni, L., Cooper, E., Krainer, A. R., Smith, H. C., Two proteins essential for apolipoprotein B mRNA editing are expressed from a single gene through alternative splicing. J. Biol. Chem., 277:12703-09 (2002).
Davidson, N. O., Powell, L. M., Wallis, S. C. and Scott, J. Thyroid hormone modulates the introduction of a stop codon in rat liver apolipoprotein B messenger RNA. J. Biol. Chem. 263, 13482-13485 (1988) .
Dettenhofer, M., S. Cen, B. A. Carlson, L. Kleiman, and X. F. Yu, Association of human immunodeficiency virus type 1 Vif with RNA and its role in reverse transcription. J Virol, 74(19):8938-45 (2000).
Doi, T., K. Kinoshita, M. Ikegawa, M. Muramatsu, and T. Honjo, Inaugural Article: De novo protein synthesis is required for the activation-induced cytidine deaminase fuinction in class-switch recombination Proc Natl Acad Sci USA 100(5):2634-8 (2003).
Driscoll, D. M., Lakhe-Reddy, S., Oleksa, L. M. and Martinez, D. Induction of RNA editing at heterologous sites by sequences in apolipoprotein B mRNA. Mol. Cell. Biol. 13, 7288-7294 (1993).
Driscoll, D. M. and E. Casanova, Characterization of the apolipoprotein B mRNA editing activity in enterocyte extracts. J Biol Chem. 265(35):21401-3 (1990).
Economidis, I. V. and T. Pederson, In vitro assembly of a pre-messenger ribonucleoprotein. Proc Natl Acad Sci USA, 80(14):4296-300 (1983).
Egebjerg, J., Kukekov, V. and Heinemann, S. F. Intron sequence directs RNA editing of the glutamate receptor subunit GluR2 coding sequence. Proc. Natl. Acad. Sci. U.S.A. 91, 10270-10274 (1994).
Ehrenstein, M. R. and M. S. Neuberger Deficiency in Msh2 affects the efficiency and local sequence specificity of immunoglobulin class-switch recombination: parallels with somatic hypermutation. Embo J, 18(12): p. 3484-90 (1999).
Eisenberg, D., R. Luthy, and J. U. Bowie, VERIFY3D: assessment of protein models with three-dimensional profiles. Methods Enzymol. 277:396-404 (1997).
Faham, M., S. Baharloo, S. Tomitaka, J. DeYoungand N. B. Freimer, Mismatch repair detection (MRD): high-throughput scanning for DNA variations. Hum Mol Genet. 10(16):1657-64 (2001).
Fisher, A. G., B. Ensoli, L. Ivanoff, M. Chamberlain, S. Petteway, L. Ratner, R. C. Gallo, and F. Wong-Staal, The sor gene of HIV-1 is required for efficient virus transmission in vitro. Science. 237(4817):888-93 (1987).
Fisher, C. L. and Pei, K. P. Modification of a PCR-based site-directed mutagenesis method. BioTechniques 23, 570-574 (1997).
Fugmann, S. D. and Schatz, D. G. Immunology. One AID to unite them all. Science. 295:1244-5 (2002).
Funahashi, T., Giannoni, F., DePaoli, A. M., Skarosi, S. F. and Davidson, N. O. Tissue-specific, developmental and nutritional regulation of the gene encoding the catalytic subunit of the rat apoB mRNA editing enzyme: functional role in the modulation of apoB mRNA editing. J. Lipid Res. 36:414-428 (1995).
Gaddis, N. C., Certova, E., Sheehy, A. M., Henderson, L. E. and Malim, M. H. Comprehensive investigation of the molecular defect in vif-deficient human immunodeficiency virus type 1 virions. J. Virol. 77(10): 5810-5820 (2003).
Gerber, A., H. Grosjean, T. Melcher, and W. Keller Tadlp, a yeast tRNA-specific adenosine deaminase, is related to the mammalian pre-mRNA editing enzymes ADAR1 and ADAR2. Embo J. 17(16):4780-9 (1998).
Gerber, A. P. and Keller, W. RNA editing by base deamination: more enzymes, more targets, new mysteries. TIBS 26:376-384 (2001).
Gerber, A. P. and W. Keller An adenosine deaminase that generates inosine at the wobble position of tRNAs. Science 286(5442):1146-9 (1999).
Giannoni, F., Bonen, D. K., Funahashi, T., Hadjiagapiou, C., Burant, C. F. and Davidson, N. O. Complementation of apolipoprotein B mRNA editing by human liver accompanied by secretion of apolipoprotein B48. J. Biol. Chem. 269:5932-5936 (1994).
Giannoni, F., Chou, S. C., Skarosi, S. F., Verp, M. S., Field, F. J., Coleman, R. A. and Davidson, N. O. Developmental regulation of the catalytic subunit of the apoB mRNA editing enzyme (APOBEC-1) in human small intestine. J. Lipid Res. 36:1664-1675 (1995).
Gott, J. M. and Emeson, R. B. Functions and mechanisms of RNA editing. Annu. Rev. Genet. 34, 499-531 (2000).
Greeve, J., Altkemper, I., Dieterich, J-H., Greten, H. and Winder, E. (1993) Apolipoprotein B mRNA editing in 12 different mammalian species: hepatic expression is reflected in low concentrations of apoB-containing plasma lipoproteins. J. Lipid Res. 34:1367-1383 (2000).
Greeve, J., Lellek, H., Rautenberg, P. and Greten, H. Inhibition of the apolipoprotein B mRNA editing enzyme-complex by hnRNP C1 protein and 40S hnRNP complexes. Biol. Chem. 379:1063-1073 (1998).
Grosjean, H. and Benne, R. Modification and Editing of RNA. ASM. Press, Washington D.C. (1998)
Harris, R. S., Bishop, K. N., Sheehy, A. M., Craig, H. M., Petersen-Mahrt, S. K., Watt, I. N., Neuberger, M. S., and Malim, M. H. DNA deamination mediates innate immunity to retroviral infection. Cell. 113:803-809 (2003).
Harris, R. S., S. K. Petersen-Mahrt, et al. (2002). “RNA editing enzyme APOBEC1 and some of its homologs can act as DNA mutators.” Mol Cell 10(5): 1247-53.
Harris, R. S., S. K. Petersen-Mahrt, and M. S. Neuberger, RNA editing enzyme APOBEC1 and some of its homologs can act as DNA mutators. Mol Cell. 10(5): 1247-53 (2002).
Harris, S. G., Sabio, I., Mayer, E., Steinburg, M. F., Backus, J. W., Sparks, J. D., Sparks, C. E. and Smith, H. C. Extract-specific heterogeneity in high-order complexes containing apolipoprotein B mRNA editing activity and RNA-binding proteins. J. Biol. Chem. 268(10):7382-7392 (1993).
Harris, S. G. and Smith, H. C. In vitro apoB mRNA editing activity can be modulated by fasting and refeeding rats with a high carbohydrate diet. Biochem. Biophys. Res. Commun. 183(2):899-903 (1992).
Henzler, T., Harmache, A., Herrmann, H., Spring, H., Suzan, M., Audoly, G., Panek, T. and Bosch, V. Fully functional, naturally occurring and C-terminally truncated variant human immunodeficiency virus (HIV) Vif does not bind to HIV Gag but influences intermediate filament structure. J. Gen Virol. 82:561-573 (2001).
Hersberger, M. and Innerarity, T. L. Two efficiency elements flanking the editing site of cytidine 6666 in the apolipoprotein B mRNA support mooring dependent editing. J. Biol. Chem. 273:9435-9442 (1998).
Hersberger, M., Patarroyo-White, S., Arnold, K. S. and Innerarity, T. L. Phylogenetic analysis of the apolipoprotein B mRNA editing region. Evidence for a secondary structure between the mooring sequence and the 3′ efficiency element. J. Biol. Chem. 274, 34590-34597 (1999).
Higuchi, M., Maas, S., Single, F. N., Hartner, J., Rozov, A., Burnashev, N., Feldmeyer, D., Sprengel, R. and Seeburg, P. H. Point mutation in an AMPA receptor gene rescues lethality in mice deficient in the RNA editing enzyme ADAR2. Nature (London) 405:78-81 (2000).
Higuchi, M., Single, F. N., Köhler, M., Sommer, B., Sprengel, R. and Seeburg, P. H. RNA editing of AMPA receptor subunit GluR-B: a base-paired intron-exon structure determines position and efficiency. Cell 75:1361-1370 (1993).
Hilleren, P. and R. Parker, mRNA surveillance in eukaryotes: kinetic proofreading of proper translation termination as assessed by mRNP domain organization? RNA. 5(6):711-9 (1999).
Hirano, K. I., Young, S. G., Farese, R. V., Ng, J., Sande, E., Warburton, C., Powell-Braxton, L. M. and Davidson, N. O. Targeted disruption of the mouse apobec-1 gene abolishes apoB mRNA editing and eliminates ApoB48. J. Biol. Chem. 271, 9887-9890 (1996).
Honjo, T., et al. Molecular Mechanism of Class Switch Recombination: Linkage with Somatic Hypermutation. Annu Rev Immunol. 20:165-96 (2002).
Hu, B. T., S. C. Lee, E. Marin, D. H. Ryanand R. A. Insel, Telomerase is up-regulated in human germinal center B cells in vivo and can be re-expressed in memory B cells activated in vitro. J Immunol. 159(3):1068-71 (1997). Hwang, J. T., K. A. Tallman, and M. M. Greenberg, The reactivity of the 2-deoxyribonolactone lesion in single-stranded DNA and its implication in reaction mechanisms of DNA damage and repair. Nucleic Acids Res, 27(19):3805-10 (1999).
Inui, Y., Giannoni, F., Funahashi, T. and Davidson, N. O. REPR and complementation factor(s) interact to modulate rat apolipoprotein B mRNA editing in response to alterations in cellular cholesterol flux. J. Lipid Res. 35, 1477-1489 (1994).
Jarmuz, A., et al. An Anthropoid-Specific Locus of Orphan C to U RNA-Editing Enzymes on Chromosome 22. Genomics. 79:285-96 (2002).
Johansson E, Mejlhede N, Neuhard J, Larsen S. Crystal structure of the tetrameric cytidine deaminase from Bacillus subtilis at 2.0 Å resolution. Biochem. 41(8):2563-70 (2002)
Jones, T. A., J. Y. Zou, S. W. Cowan, and Kjeldgaard, Improved methods for building protein models in electron density maps and the location of errors in these models. Acta Crystallogr A. 47 (Pt 2):110-9 (1991).
Kabsch, W., A solution for the best rotation to relate two sets of vectors. Acta. Crystallogr., A32:922-923 (1976).
Kataoka, N., Yong, J., Kim, V. N., Velazquez, F., Perkinson, R. A., Wang, F. and Dreyfuss, G. Pre-mRNA splicing imprints mRNA in the nucleus with a novel RNA-binding protein that persists in the cytoplasm. Mol. Cell 6:673-682 (2000).
Kaushik, N. and V. N. Pandey, PNA targeting the PBS and A-loop sequences of HIV-1 genome destabilizes packaged tRNA3(Lys) in the virions and inhibits HIV-1 replication. Virology. 303(2):297-308 (2002).
Keegan, L. P., A. P. Gerber, J. Brindle, R. Leemans, A. Gallo, W. Keller, and M. A. O'Connell, The properties of a tRNA-specific adenosine deaminase from Drosophila melanogaster support an evolutionary link between pre-mRNA editing and tRNA modification. Mol Cell Biol 20(3):825-33 (2000).
Keegan, L. P., et al. The many roles of an RNA editor. Nat Rev Genet. 2:869-78 (2001).
Keller, W., J. Wolf, and A. Gerber, Editing of messenger RNA precursors and of tRNAs by adenosine to inosine conversion. FEBS Lett, 452(1-2):71-6. (1999).
Khan, M. A., Aberham, C., Kao, S., Akari, H., Gorelick, R., Bour, S. and Strebel, K. Human immunodeficiency virus type 1 Vif protein is packaged into the nucleoprotein complex through an interaction with viral genomic RNA. J. Virol. 75(16):7252:7265 (2001).
Kleiman, L., tRNA(Lys3): the primer tRNA for reverse transcription in HIV-1. IUBMB Life 53(2):107-14 (2002).
Kohler, M., Bumashev, N., Sakmann, B. and Seeburg, P. H. Determinants of Ca ²⁺ permeability in both TM1 and TM2 of high affinity kainate receptor channels : diversity by RNA editing. Neuron 10:491-500 (1993).
Krogh, A., Brown, M., Mian, I. S., Sjolander, K. and Haussler, D. Hidden Markov models in computational biology. Applications to protein modeling, J Mol Biol. 235:1501-31 (1994).
Kumar, M. and G. G. Carmichael Nuclear antisense RNA induces extensive adenosine modifications and nuclear retention of target transcripts. Proc Natl Acad Sci USA. 94(8):3542-7 (1997).
Kuyper, L. F. and C. W. Carter, Resolving crystal polymorphism by finding ‘stationary points’ from quantitative analysis of crystal growth response surfaces. J. Crystal Growth. 168:135-169 (1996).
Kuzin, I. I., J. E. Snyder, G. D. Ugine, D. Wu, S. Lee, T. J. Bushnell, R. A. Insel, F. M. Young, Bottaro, A., Tetracyclines inhibit activated B cell function. Int. Immunol. 12:921-931 (2001).
Kuzin, II, G. D. Ugine, D. Wu, F. Young, J. Chenand A. Bottaro, Normal isotype switching in B cells lacking the I mu exon splice donor site: evidence for multiple I mu-like germline transcripts. J Immunol. 164(3):1451-7 (2000).
Lau, P. P., H. J. Zhu, et al. (1994). “Dimeric structure of a human apolipoprotein B mRNA editing protein and cloning and chromosomal localization of its gene.” Proc Natl Acad Sci USA 91(18): 8522-6.
Lau, P. P., Xiong, W. J., Zhu, H. J., Chen, S. H. and Chan, L. Apolipoprotein B mRNA editing is an intranuclear event that occurs post-transcriptionally coincident with splicing and polyadenylation. J. Biol. Chem. 266:20550-20554 (1991).
Lau, P. P, Chang, B. H. J. and Chan, L. Two-hybrid cloning identifies an RNA-binding protein GRY-RBP, as a component of apobec-1 editosome. Biochem. Biophys. Res. Commun. 282(4):977-983 (2001).
Lau, P. P., Cahill, D. J., Zhu, H. J. and Chan, L. Ethanol modulates apoB mRNA editing. J. Lipid Res. 36:2069-2078 (1995).
Lau, P. P., Villanueva, H., Kobayashi, K., Nakamuta, M., Chang, H. J., Chan, L., A DnaJ protein, Apobec-1-binding protein-2, modulates apolipoprotein B mRNA editing. J. Biol. Chem. 276:46445-46452 (2001).
Lau, P. P., Zhu, H. J., Baldini, A., Charnsangavej, C. and Chan, L. Dimeric structure of a human apolipoprotein B mRNA editing protein and cloning and chromosomal localization of its gene. Proc. Natl. Acad. Sci. USA 91:8522-8526 (1994).
Lau, P. P., Zhu, H. J., Nakamuta, M. and Chan, L. Cloning of an Apobec-1-binding protein that also interacts with apolipoprotein B mRNA and evidence for its involvement in RNA editing. J. Biol. Chem. 272(3):1452-1455 (1997).
Le Hir, H., Izaurralde, E., Maquat, L. E. and Moore, M. J. (2000) The spliceosome deposits multiple proteins 20-24 nucleotides upstream of mRNA exon-exon junctions. EMBO J. 19, 6860-6869.
Lecossier, D., Bouchonnet, F., Clavel, F. and Hance, A. J. (2003) Science 300: 1112.
Lee, R. M., et al., (1998) An alternatively spliced form of apobec-1 messenger RNA is overexpressed in human colon cancer. Gastroenterology. 115:1096-103.
Lellek, H., Kirsten, R., Diehl, I., Apostel, F., Buck, F. and Greeve, J. (2000) Purification and Molecular cloning of a novel essential component of the apolipoprotein B mRNA editing Enzyme-complex. J. Biol. Chem., 275, 19848-19856.
Lesk, A. M. and C. Chothia, How different amino acid sequences deterinine similar protein structures: the structure and evolutionary dynamics of the globins. J Mol Biol 136(3):225-70 (1980).
Lewis, J. D. and Tollervey, D. (2000) Like attracts like: getting RNA processing together in the nucleus. Science 288, 1385-1389.
Liao, W., Hong, S. H., Chan, B. H. J., Rudolph, F. B., Clark, S. C. and Chan, L. (1999) APOBEC-2, a cardiac-and skeletal muscle-specific member of the cytidine deaminase supergene family. Biochem. Biophys. Res. Commun. 260, 398-404.
Liu, H., X. Wu, M. Newman, G. M. Shaw, B. H. Hahn, and J. C. Kappes, The Vif protein of human and simian immunodeficiency viruses is packaged into virions and associates with viral core structures. J Virol. 69(12):7630-8 (1995).
Liu, H. X., L. Cartegni, M. Q. Zhangand A. R. Krainer, A mechanism for exon skipping caused by nonsense or missense mutations in BRCA1 and other genes. Nat Genet, 27(1):55-8 (2001).
Liu, H. X., M. Zhangand A. R. Krainer, Identification of functional exonic splicing enhancer motifs recognized by individual SR proteins. Genes Dev, 12(13): 1998-2012 (1998).
Liu, Y. and C. E. Samuel, Mechanism of interferon action: functionally distinct RNA-binding and catalytic domains in the interferon-inducible, double-stranded RNA-specific adenosine deaminase. J Virol. 70(3):1961-8 (1996).
Liu, Y., R. B. Emeson, and C. E. Samuel, Serotonin-2C receptor pre-mRNA editing in rat brain and in vitro by splice site variants of the interferon-inducible double-stranded RNA-specific adenosine deaminase ADAR1. J Biol Chem. 274(26):8351-8 (1999).
Longacre, A. and U. Storb, A novel cytidine deaminase affects antibody diversity. Cell 102(5): 541-4 (2000).
Maas, S. and Rich, A. (2000) Changing genetic information through RNA editing. BioEssays 22, 790-802.
Maas, S., Melcher, T. and Seeburg, P. H. (1997) Mammalian RNA-dependent deaminases and edited mRNAs. Curr. Opin. Cell. Biol. 9, 343-349.
Maas, S., Melcher, T., Herb, A., Seeburg, P. H., Keller, W., Krause, S., Higuchi, M. and O'Connell, M. A. (1996). Structural requirements for RNA editing in glutamate receptor pre-mRNA by recombinant double-stranded RNA adenosine deaminase. J. Biol. Chem. 271, 12221-12226.
MacGinnitie, A. J., Anant, S. and Davidson, N. O. (1995) Mutagenesis of APOBEC-1, the catalytic subunit of the mammalian apolipoprotein B mRNA editing enzyme, reveals distinct domains that mediate cytosine nucleoside deaminase, RNA-binding, and RNA editing activity. J. Biol. Chem. 270, 14768-14775.
Madani, N. and D. Kabat, An endogenous inhibitor of human immunodeficiency virus in human lymphocytes is overcome by the viral Vifprotein. J Virol. 72(12):10251-5 (1998).
Madsen P., Anant S., Rasmussen, H. H., Gromov, P., Vorum, H., Dumanski, J. P., Tommerup, N., Collins, J. E., Wright, C. L., Dunham, I., MacGinnitie, A. J., Davidson, N. O. and Celis, J. E. Psoriasis upregulated phorbolin-1 shares structural but not functional similarity to the mRNA-editing protein apobec-1. J. Invest. Dermatol. 113(2):162-169 (1999).
Mangeat, B., Turelli, P., Caron, G., Friedli, M., Perrin, L., and Trono, D. Broad antiretroviral defense by human APOBEC3G through lethal editing of nascent reverse transcripts. Nature. Advance online publication, in press (2003).
Manis, J. P., Y. Gu, R. Lansford, E. Sonoda, R. Ferrini, L. Davidson, K. Rajewskyand F. W. Alt, Ku70 is required for late B cell development and immunoglobulin heavy chain class switching. J Exp Med. 187(12):2081-9 (1998).
Mansky, L. M., S. Preveral, L. Selig, R. Benarous, and S. Benichou, The interaction of vpr with uracil DNA glycosylase modulates the human immunodeficiency virus type 1 In vivo mutation rate. 74(15):7039-47 (2000).
Maquat, L. and Carmichael, G. G. Quality control of mRNA function. Cell 104(2):173-176 (2001).
Marinettii, G. V., Disorders of Lipid Metabolism. New York: Plenum Press (1990).
Mariani, R., Chen, D., Schrofelbauer, B., Navarro, F., Konig, R., Bollman, B., Munk, C., McMahon, H., and Landau, N. Cell 114: 21-31 (2003).
Martin, A. and M. D. Scharff, AID and mismatch repair in antibody diversification. Nat Rev Immunol. 2(8):605-14 (2002).
Martin, A., P. D. Bardwell, C. J. Woo, M. Fan, M. J. Shulmanand M. D. Scharff, Activation-induced cytidine deaminase turns on somatic hypermutation in hybridomas. Nature. 415(6873): 802-6, (2002).
McCahill, A., Lankester, D. J., Park, S., Price, N. T. and Zammit, V. A. (2000) Acute modulation of the extent of apoB mRNA editing and relative rates of synthesis of apoB48 and apoB100 in cultured rat hepatocytes by osmotic and other stresses. Molec. Cell. Biochem. 208, 77-87.
Mehta, A., Driscoll, D. M. Identification of Domains in APOBEC-1 Complementation Factor Required for RNA Binding and Apolipoprotein B mRNA editing. RNA. 8:69-82 (2002).
Mehta, A., Kinter, M. T., Sherman, N. E. and Driscoll, D. M. Molecular cloning of apobec-1 complementation factor, a novel RNA-binding protein involved in the editing of apolipoprotein B mRNA, Mol Cell Biol. 20:1846-54 (2000).
Mian, I. S., Moser, M. J., Holley, W. R. and Chattejee, A. Statistical modeling and phylogenetic analysis of a deaminase domain, J Comput. Biol. 5: 57-72 (1998).
Minegishi, Y., A. Lavoie, et al. (2000). “Mutations in activation-induced cytidine deaminase in patients with hyper IgM. syndrome.” Clin Immunol 97(3): 203-10.
Minegishi, Y., et al., (2000) Mutations in activation-induced cytidine deaminase in patients with hyper IgM. syndrome. Clin Immunol. 97:203-10.
Morrison, J. R., Paszty, C., Stevens, M. E., Hughes, S. D., Forte, T. and Scott, J. (1996) ApoB RNA editing enzyme-deficient mice are viable despite alterations in lipoprotein metabolism. Proc. Natl. Acad. Sci. USA 93, 7154-7159.
Mukhopadhyay, D., S. Anant, R. M. Lee, S. Kennedy, D. Viskochiland N. O. Davidson, C→U editing of neurofibromatosis 1 mRNA occurs in tumors that express both the type II transcript and apobec-1, the catalytic subunit of the apolipoprotein B mRNA-editing enzyme. Am J Hum Genet. 70(1):38-50 (2002).
Muramatsu, M., K. Kinoshita, et al. (2000). “Class switch recombination and hypermutation require activation-induced cytidine deaminase (AID), a potential RNA editing enzyme.” Cell 102(5): 553-63.
Muramatsu, M., Kinoshita, K., Fagarasan, S., Yamada, S., Shinkai, Y. and Honjo, T. (2000) Class switch recognition and hypermutation require activation-induced cytidine deaminase (AID), a potential RNA editing enzyme. Cell 102, 553-564.
Muramatsu, M., Sankaranand, V. S., Anant, S., Sugai, M., Kinoshita, K., Davidson, N. O. and Honjo, T. Specific expression of activation-induced cytidine deaminase (AID), a novel member of the RNA-editing deaminases family in germinal center B cells. J. Biol. Chem. 274:18470-18476 (1999).
Muramatsu, M., V. S. Sankaranand, et al. (1999). “Specific expression of activation-induced cytidine deaminase (AID), a novel member of the RNA-editing deaminase family in germinal center B cells.” J Biol Chem 274(26): 18470-6.
Muschen, M., K. Rajewsky, M. Kronkeand R. Kuppers, The origin of CD95-gene mutations in B-cell lymphoma. Trends Immunol, 2002. 23(2): p. 75-80.
Muto, T., M. Muramatsu, et al. (2000). “Isolation, tissue distribution, and chromosomal localization of the human activation-induced cytidine deaminase (AID) gene.” Genomics 68(1): 85-8.
Nagaoka, H., M. Muramatsu, N. Yamnamura, K. Kinoshitaand T. Honjo, Activation-induced deaminase (AID)-directed hypermutation in the immunoglobulin Smu region: implication of AID involvement in a common step of class switch recombination and somatic hypermutation. J Exp Med, 2002. 195(4): p. 529-34.
Nakamuta, M., Chang, B. H. J., Zsignond, E., Kobayashi, K., Lei, H., Ishida, B. Y., Oka, K., Li, E. and Chan, L. (1996) Complete phenotypic characterization of apobec-1 knockout mice with a wild-type genetic background and a human apoB transgenic background, and restoration of apoB mRNA editing by somatic gene transfer of APOBEC-1. J. Biol. Chem. 271, 25981-25988.
Navaratnam, N., Bhattacharya, S., Fujino, T., Patel, D., Jarmuz, A. L. and Scott, J. Evolutionary origins of apoB mRNA editing: catalysis by a cytidine deaminase that has acquired a novel RNA-binding motif at its active site. Cell 81, 187-195 (1995).
Navaratnam, N., D., Patel, R. R., Shah, J. C., Greeve L. M., Powell, T. J., Knott, J., Scott, An additional editing site is present in apolipoprotein B mRNA. Nucleic Acids Res., 19:1741-1744 (1991).
Navaratnam, N., Fujino, T., Bayliss, J., Jarmuz, A., How, A. Richardson, N., Somasekaram, A. Bhattacharya, S., Carter, C. & Scott, J. Escherichia coli cytidine deaminase provides a molecular model for ApoB RNA editing and a mechanism for RNA substrate recognition JMB 275:695-714 (1998).
Navaratnam, N., R. Shah, D. Patel, V. Fayand J. Scott, Apolipoprotein B mRNA editing is associated with UV crosslinking of proteins to the editing site. Proc Natl Acad Sci USA. 90(1):222-6 (1993).
Neuberger, M. S., Harris, R. S., Di Noia, J., and Petersen-Mahrt, S. K. Immunity through DNA deamination. Trends in Biochemical Sciences. Advanced online publication, in press (2003).
Neumann, J. R., Morency, C. A. and Russian, K. O. A novel rapid assay for chloramphenicol acetyltransferase gene expression. BioTechniques 5: 444-448 (1987).
O'Connell, M. A. RNA Editing: Rewriting Receptors. Current Biology 7:R437-R439 (1997).
Ohagen, A. and D. Gabuzda, Role of Vif in stability of the human immunodeficiency virus type 1 core. J Virol, 74(23): 11055-66 (2000).
Oka, K., Kobayashi, K., Sullivan, M., Martinez, J., Teng, B. B., Ishimura-Oka, K. and Chan, L. Tissue-specific inhibition of apoB B mRNA editing in the liver by adenovirus-mediated transfer of a dominant negative mutant APOBEC-1 leads to increased low density lipoprotein in mice. J. Biol. Chem. 272(3):1456-1460 (1997).
Okazaki, I. M., et al. The AID enzyme induces class switch recombination in fibroblasts. Nature. 416:340-5 (2002).
Paddison, P. J., A. A. Caudy, E. Bernstein, G. J. Hannonand D. S. Conklin, Short hairpin RNAs (shRNAs) induce sequence-specific silencing in mammalian cells. Genes Dev, 2002. 16(8):948-58.
Paddison, P. J., A. A. Caudyand G. J. Hannon, Stable suppression of gene expression by RNAi in mammalian cells. Proc Natl Acad Sci USA, 2002. 99(3): p. 1443-8.
Papavasiliou, F. N. and D. G. Schatz Cell-cycle-regulated DNA double-stranded breaks in somatic hypennutation of immunoglobulin genes. Nature 408(6809):216-21 (2000).
Papavasiliou, F. N. and D. G. Schatz The Activation-induced Deaminase Functions in a Postcleavage Step of the Somatic Hypermutation Process. J Exp Med 195(9):1193-1198 (2002).
Petersen-Mahrt, S. K., et al., AID mutates E. coli suggesting a DNA deamination mechanism for antibody diversification. Nature 418:99-104 (2002).
Pham, P., Bransteitter, R., Petruska, J. and Goodman, M. F. Processive AID-catalyzed cytosine deamination on single-stranded DNA simulates somatic hypermutation. Nature Advanced online publication, in press.
Phung, T. L., Sowden, M. P., Sparks, J. D., Sparks, C. E. and Smith, H. C. (1996) Regulation of hepatic apoB RNA editing in the genetically obese Zucker rat. Metabolism 45, 1056-1058.
Polson, A. G., B. L. Bass, and J. L. Casey, RNA editing of hepatitis delta virus antigenome by dsRNA-adenosine deaminase. Nature 380(6573):454-6 (1996).
Potterton, E., S. McNicholas, E. Krissinel, K. Cowtan, and M. Noble, The CCP4 molecular-graphics project. Acta Crystallogr D Biol Crystallogr. 58(Pt 11):1955-7 (2002).
Powell, L. M., Wallis, S. C., Pease, R. J., Edwards, Y. H., Knott, T. J. and Scott, J. (1987) A novel form of tissue-specific RNA processing produces apolipoprotein-B48 in intestine. Cell 50, 831-840.
Puck, J. M., A disease gene for autosomal hyper-IgM. syndrome: more genes associated with more immunodeficiencies. Clin Immunol,(2000). 97(3): p. 191-2
Rada, C., et al., (2002) AID-GFP chimeric protein increases hypermutation of Ig genes with no evidence of nuclear localization. Proc. Natl. Acad. Sci USA. 99:7003-7008
Ramiro, A. R., P. Stavropoulos, M. Jankovic, and M. C. Nussenzweig, Transcription enhances AID-mediated cytidine deamination by exposing single-stranded DNA on the nontemplate strand. Nat Immunol (2003).
Renda, M. J., J. D. Rosenblatt, E. Klimatcheva, L. M. Demeter, R. A. Bambara, and V. Planelles, Mutation of the methylated tRNA(Lys)(3) residue A58 disrupts reverse transcription and inhibits replication of human immunodeficiency virus type 1. J Virol 75(20):9671-8 (2001).
Revy, P, Muto, R., Levy, Y., Geissmann, f., Plebani, A., Sanal, O., Catalan, N., Forveille, M., Dufourcq-Lagelouse, R., Gennery, A., Tezcan, I., Ersoy, F., Kayserili, H., Ugazio, A. G., Brousse, N., Muramatsu, M., Notarangelo, L. D., Kinoshita, K., Honjo, T., Fisher, A. and Durandy, A. Activation-induced cytidine deaminase (AID) deficiency causes the autosomal recessive form of the hyper-IgM. syndrome (HIGM2). Cell 102,(5):565-576 (2000).
Revy, P., T. Muto, et al. (2000). “Activation-induced cytidine deaminase (AID) deficiency causes the autosomal recessive form of the Hyper-IgM. syndrome (HIGM2).” Cell 102(5): 565-75.
Richardson, N., Navaratnam, N. and Scott, J. (1998) Secondary structure for the apolipoprotein B mRNA editing site. AU binding proteins interact with a stem loop. J. Biol Chem. 273, 31707-31717.
Robberson, B. L., Cote, G. J. and Berget, S. M. (1990) Exon definition may facilitate splice site selection in RNAs with multiple exons. Mol. Cell. Biol. 10, 1084-1094.
Rolink, A., F. Melchersand J. Andersson, The SCID but not the RAG-2 gene product is required for S mu-S epsilon heavy chain class switching. Immunity, 1996. 5(4): p. 319-30.
Rueter, S. M. and Emeson, R. B. (1998) Adenosine-to-inosine conversion in mRNA. In Modification and Editing of RNA (Grosjean, H. and Benne, R., eds.), pp. 343-361, American Society for Microbiology Press, Washington.
Rueter, S. M., Dawson, T. R. and Emeson, R. B. (1999) Regulation of alternative splicing by RNA editing. Nature 399, 75-80.
Sakashita, E. and H. Sakamoto, Protein-RNA and protein-protein interactions of the Drosophila sex-lethal mediated by its RNA-binding domains. Journal of Biochemistry, 1996. 120(5): p. 1028-33.
Sale, J. E., D. M. Calandrini, M. Takata, S. Takedaand M. S. Neuberger, Ablation of XRCC2/3 transforms immunoglobulin V gene conversion into somatic hypermutation. Nature, 2001. 412(6850): p. 921-6.
Sali, A., L. Potterton, F. Yuan, H. van Vlijmen, and M. Karplus, Evaluation of comparative protein modeling by MODELLER Proteins. 23(3): p. 318-26 (1995).
Schock, D., Kuo, S. R., Steinburg, M. F., Bolognino, M., Sparks, J. D., Sparks, C. E. and Smith, H. C. (1996). An auxiliary factor containing a 240 kDa protein is involved in apoB RNA editing. Proc. Natl. Acad. Sci. USA 93, 1097-1102.
Scott, J. (1989) The molecular and cell biology of apolipoprotein-B.J. Mol. Med. 6, 65-80.
Seeburg, P. H., Higuchi, M. and Sprengel, R. (1998) RNA editing of brain glutamate receptor channels : mechanism and physiology. Brain Res. Rev. 26, 217-229.
Selig, L., S. Benichou, M. E. Rogel, L. I. Wu, M. A. Vodicka, J. Sire, R. Benarous, and M. Emerman, Uracil DNA glycosylase specifically interacts with Vpr of both human immunodeficiency virus type 1 and simian immunodeficiency virus of sooty mangabeys, but binding does not correlate with cell cycle arrest. J Virol. 71(6):4842-6. (1997).
Shah, R. R., Knott, T. J., Legros, J. E., Navaratnam, N., Greeve, J. C. and Scott, J. Sequence requirements for the editing of apolipoprotein B mRNA. J. Biol. Chem. 266, 16301-16304 (1991).
Sheehy, A. M., et al., (2002) Isolation of a human gene that inhibits HIV-1 infection and is suppressed by the viral Vif protein. Nature. 418:646-650.
Siddiqui, J. F. M., Van Mater, D., Sowden, M. P. and Smith, H. C. (1999) Disproportionate relationship between APOBEC-1 expression and apolipoprotein B mRNA editing activity. Exp. Cell Res. 252(1):154-164.
Simon, J. H. and M. H. Malim. The human immunodeficiency virus type 1 Vif protein modulates the postpentration stability of viral nucleoprotein complexes. J Virol. 70(8):5297-305 (1996).
Simon, J. H., N. C. Gaddis, R. A. Fouchier, and M. H. Malim, Evidence for a newly discovered cellular anti-HIV-1 phenotype. Nat Med. 4(12):1397-400 (1998).
Simpson, L. and Emeson, R. B. (1996) RNA editing. Annu. Rev. Neurosci. 19, 27-52.
Skuse, G. R., A. J. Cappione, M. Sowden, L. J. Methenyand H. C. Smith, The neurofibromatosis type I messenger RNA undergoes base-modification RNA editing. Nucleic Acids Res, 1996. 24(3): p. 478-85
Smith, H. C., Kuo, S. R., Backus, J. W., Harris, S. G., Sparks, C. E. and Sparks, J. D. (1991) In vitro mRNA editing: identification of a 27 S editing complex. Proc. Natl. Acad. Sci. U.S.A. 88, 1489-1493.
Smith, H. C. (1993) Apo B mRNA editing: the sequence to the event. Seminars in Cell Biology (Stuart, K., ed.) Saunders Sci. Publications/Academic Press, London, 4, 267-278.
Smith, H. C. and Sowden, M. P. (1996) Base modification RNA editing Trends in Genetics 12, 418-424.
Smith, H. C., Analysis of protein complexes assembled on apolipoprotein B mRNA for mooring sequence-dependent RNA editing. Methods, 1998. 15(1): p. 27-39.
Smith, H. C., Gott, J. M. and Hanson, M. R. (1997) A guide to RNA editing. RNA, 3, 1105-1123.
Sohail, A., Klapacz, J., Samaranayake, M., Ullah, A. and Bhagwat, A. Human activation-induced cytidine deaminase causes transcript-dependent, strand-biased C to U deaminations. Nucleic Acids. Res. 31(12):2990-2994 (2003).
Sova, P. and D. J. Volsky, Efficiency of viral DNA synthesis during infection of permissive and nonpermissive cells with vif-negative human immunodeficiency virus type 1. J Virol. 67(10): 6322-6 (1993).
Sowden, M. P., Hamm, J. K. and Smith, H. C. (1996) Over-expression of APOBEC-I results in mooring sequence dependent promiscuous RNA editing. J. Biol. Chem. 271(6):3011-3017.
Sowden, M. P., Harrison, S. M., Ashfield, R. A., Kingsman, A. J. and Kingsman, S. M. (1989) Multiple cooperative interactions constrain BPV-1 E2 dependent activation of transcription. Nucleic Acids Res. 17, 2959-2972.
Sowden, M. P. and H. C. Smith, Commitment of apolipoprotein B RNA to the splicing pathway regulates cytidine-to-uridine editing-site utilization. Biochem J, 2001. 359(Pt 3): p. 697-705.
Sowden, M. P., Ballatori, N., de Mesy Jensen, K. L., Hamilton Reed, L., Smith, H. C., The editosome for cytidine to uridine mRNA editing has a native complexity of 27S: identification of intracellular domains containing active and inactive editing factors. J. Cell Science, 2002. 115: p. 1027-1039
Sowden, M. P., Eagleton, M. J. and Smith, H. C. (1998). ApoB RNA sequence 3′ of the mooring sequence and cellular sources of auxiliary factors determine the location and extent of promiscuous editing. Nucleic Acids Res. 26, 1644-1652.
Sowden, M. P., Hamm, J. K., Spinelli, S. and Smith, H. C. (1996) Determinants involved in regulating the proportion of edited apolipoprotein B RNAs. RNA 2(3):274-288.
Spector, D. (1993) Macromolecular domains within the cell nucleus. Annu. Rev. Cell Biol. 9, 265-315.
Steinburg, M. F., Schock, D., Backus, J. W. and Smith, H. C. (1999) Tissue-specific differences in the role of RNA 3′ of the apolipoprotein B mRNA mooring sequence in editosome assembly. Biochem. Biophys. Res. Commun. 263(1):81-86.
Strebel, K., D. Daugherty, K. Clouse, D. Cohen, T. Folks, and M. A. Martin, The HIV ‘A’ (sor) gene product is essentialfor virus infectivity. Nature. 328(6132): p. 728-30 (1987).
Taagepera, S., McDonald, D., Loeb, J. E., Whitaker, L. L., McElroy, A. K., Wang, J. Y. J. and Hope, T. J. (1998) Nuclear-cytoplasmic shuttling of C-ABL tyrosine kinase. Proc. Natl. Acad. Sci. U.S.A. 95, 7457-7462.
Teng, B. and N. O. Davidson, Evolution of intestinal apolipoprotein B mRNA editing. Chicken apolipoprotein B mRNA is not edited, but chicken enterocytes contain in vitro editing enhancement factor(s). J Biol Chem, 267(29): 21265-72 1992.
Teng, B., Burant, C. F. and Davidson, N. O. Molecular cloning of an apolipoprotein B messenger RNA editing protein, Science, 260:1816-1819 (1993).
Teng, B. B., S. Ochsner, Q. Zhang, K.V. Soman, P. P. Lau, and L. Chan, Mutational analysis of apolipoprotein B mRNA editing enzyme (APOBEC1). structure-function relationships of RNA editing and dimerization J Lipid Res, 40(4):623-35 (1999).
Van Mater, D., Sowden, M. P., Cianci, J., Sparks, J. D., Sparks, C. E., Ballitori, N. and Smith, H. C. (1998). Ethanol increases apoB mRNA editing in rat primary hepatocyte and McArdle cells. Biochem. Biophys Res. Commun. 252, 334-339.
Van Parijs, L., Y. Refaeli, J. D. Lord, B. H. Nelson, A. K. Abbasand D. Baltimore, Uncoupling IL-2 signals that regulate T cell proliferation, survival, and Fas-mediated activation-induced cell death. Immunity, 1999. 11(3): p. 281-8.
von Schwedler, U., J. Song, C. Aiken, and D. Trono, Vif is crucial for human immunodeficiency virus type 1 proviral DNA synthesis in infected cells. J Virol. 67(8): 4945-55 (1993).
von Wronski, M. A., Hirano, K. I., Cagen, L. M., Wilcox, H. G., Raghow, R., Thorugate, F. E., Heimberg, M., Davidson, N. O. and Elam, M. B. (1998). Insulin increases expression of apobec-1, the catalytic subunit of the apoB B mRNA editing complex in rat hepatocytes. Metabolism Clinical & Exp. 7, 869-873.
Wedekind, J. E., G. S. Dance, M. P. Sowden, and H. C. Smith, Messenger RNA editing in mammals: new members of the APOBEC family seeking roles in the family business. Trends Genet 19(4):207-16 (2003).
Wedekind, J. E., X. Kefang, G. S. Dance, M. P. Sowden, and H. C. Smith, The structure ofyeast Cdd1 provides insight into the molecular details of the mRNA editase APOBEC-1. (2003—In preparation).
Winn, M. D. An overview of the CCP4 project in protein crystallography: an example of a collaborative project. J Synchrotron Radiat. 10(Pt 1):23-5 (2003).
Wu, J. H., Semenkovish, C. F., Chen, S. H., Li, W. H. and Chan, L. (1990). ApoB mRNA editing: validation of a sensitive assay and developmental biology of RNA editing in the rat. J. Biol. Chem. 265, 12312-12316.
Yamanaka, S., Balestra, M., Ferrell, L., Fan, J., Arnold, K. S., Taylor, S., Taylor, J. M. and Innerarity, T. L. (1995). Apolipoprotein B mRNA-editing protein induces hepatocellular carcinoma and dysplasia in transgenic animals. Proc. Natl. Acad. Sci. USA 92, 8483-8487.
Yamanaka, S., K. S. Poksay, D. M. Driscoll, Innerarity, T. L., Hyperediting of multiple cytidines of apolipoprotein B mRNA by APOBEC-1 requires auxiliary protein(s) but not a mooring sequence motif. J. Biol. Chem. 271:11506-11510 (1996).
Yamanaka, S., Poksay, K. S., Balestra, M. E., Zeng, G. Q. and Innerarity, T. L. (1994) Cloning and mutagenesis of the rabbit apoB mRNA editing protein. J. Biol. Chem. 269, 21725-21734.
Yamanaka, S., Poksay, K. S., Arnold, K. S. and Innerarity, T. L. A novel translational repressor mRNA is edited extensively in livers containing tumors caused by the transgene expression of the apoB mRNA-editing enzyme. Genes Dev., 11, 321-33 (1997).
Yang, B., Gao, L., Li, L., Lu, Z., Fan, X., Patel, C. A., Pomerantz, R. J., DuBois, G. C. and Zhang, H. Potent suppression of viral infectivity by the peptides that inhibit multimerizations of human immunodeficiency virus type 1 (HIV-1) vif proteins. J. Biol Chem. 278(8):6596-6602 (2002).
Yang, Y. and Smith, H. C. (1996) In vitro reconstitution of apolipoprotein B RNA editing activity from recombinant APOBEC-1 and McArdle cell extracts. Biochem. Biophys. Res. Commun. 218, 797-801.
Yang, Y., Ballatori, N., Smith, H. C., Synthesis and secretion of the atherogenic risk factor apoB100 is reduced through TAT-mediated protein transduction of an mRNA editase into hepatocytes. Molec. Pharm. 61:269-276 (2002).
Yang, Y., Kovalski, K. and Smith, H. C. (1997) Partial characterization of the auxiliary factors involved in apoB mRNA editing through APOBEC-1 affinity chromatography, J Biol. Chem., 272, 27700-27706.
Yang, Y., M. P., Sowden Y., Yang, H. C., Smith, Intracellular Trafficking Determinants in APOBEC-1, the Catalytic Subunit for Cytidine to Uridine Editing of Apolipoprotein B mRNA. Exp. Cell Res. 267:153-164 (2001).
Yang, Y., Sowden, M. P. and Smith, H. C. (2000) Induction of cytidine to uridine editing on cytoplasmic apolipoprotein B mRNA by overexpressing APOBEC-1. J. Biol. Chem. 275(30):22663-22669.
Yang, Y., Yang, Y. and Smith, H. C. (1997) Multiple protein domains determine the cell type-specific nuclear distribution of the catalytic subunit required for apolipoprotein B mRNA editing. Proc. Natl. Acad. Sci. U.S.A. 94, 13075-13080.
Yoshikawa, K. et al. AID enzyme-induced hypermutation in an actively transcribed gene in fibroblasts. Science. 296:2033-2036 (2002).
Yoshikawa, K., Okazaki, I. M., Eto, T., Kinoshita, K., Muramatsu, M., Nagaoka, H., Honjo, T. (2002). “AID enzyme-induced hypermutation in an actively transcribed gene in fibroblasts.” Science 296: 2033-2036.
Yu, Q. and C. D. Morrow, Essential regions of the tRNA primer required for HIV-1 infectivity. Nucleic Acids Res. 28(23):4783-9 (2000).
U.S. patent application Publication No. 20030013844 Jan. 16, 2003. Zhang, H., Pomerantz, Roger J. and Yang, Bin; Thomas Jefferson University. Multimerization of HIV-1 Vif protein as a therapeutic target.

1. A method of screening for a Vif antagonist, comprising:
(a) contacting a Vif molecule with a test compound;
(b) detecting binding between the Vif molecule and the test compound; and
(c) screening the test compound that binds the Vif molecule for suppression of viral infectivity, suppression of viral infectivity by the test compound indicating the test compound is a Vif antagonist.

2. The method of claim 1, wherein the viral infectivity is HIV-1 infectivity.

3. The method of claim 1, wherein the ability to suppress viral infectivity is measured by contacting the test compound with one or more cytidine deaminase-positive cells, in the presence of HIV-1 virus expressing Vif.

4. The method of claim 3, wherein the cytidine deaminase-positive cells are CEM15 positive cells.

5. A method of making a Vif antagonist, comprising:
(a) identifying the Vif antagonist of claim 1; and
(b) modifying the Vif antagonist to enhance suppression of viral infectivity.

6. A method of making a Vif antagonist, comprising:
(a) identifying the Vif antagonist of claim 1; and
(b) modifying the Vif antagonist to lower biotoxicity,

7. The method of claim 1, wherein the Vif molecule is linked to a reporter.

8. The method of claim 7, wherein the reporter is luciferase.

9. The method of claim 7, wherein the reporter is GFP.

10. The method of claim 7, wherein the reporter is RFP.

11. The method of claim 7, wherein the reporter is FITC.

12. The method of claim 7, wherein the Vif molecule and the reporter form a chimera.

13. The method of claim 7, wherein the Vif molecule comprises SEQ ID NO: 1.

14. The method of claim 13, wherein the Vif molecule has 80% or greater homology to SEQ ID NO: 1.

15. The method of claim 1, wherein a plurality of test compounds are contacted with Vif molecules in a high throughput assay system.

16. The method of claim 15, wherein the high throughput assay system comprises an immobilized array of test compounds.

17. The method of claim 15, wherein the high throughput assay system comprises an immobilized array of Vif molecules.

18. A Vif antagonist identified by the method of claim 1.

19. A Vif antagonist made by the method of claim 7.

20. A Vif antagonist made by the method of claim 8.

21. A method of screening for cytidine deaminase activators, comprising:
(a) contacting a cytidine deaminase molecule with a test compound;
(b) detecting binding between the cytidine deaminase molecule and the test compound;
(c) screening the test compound that binds the cytidine deaminase molecule to identify a selected cytidine deaminase function, the presence of the selected function indicating a cytidine deaminase activator.

22. The method of claim 21, wherein the selected function of the cytidine deaminase is suppression of viral infectivity.

23. The method of claim 21, wherein the cytidine deaminase molecule is CEM15.

24. The method of claim 21, wherein the selected function of CEM15 is deoxycytidine mutation to deoxyuridine mutation in the first strand of cDNA of HIV-1 during or subsequent to its synthesis by reverse transcriptase.

25. The method of claim 21, wherein the selected function of CEM15 is decreased by binding to the test compound and cytidine to uridine editing of mRNA or deoxycytidine to deoxyuridine mutation of DNA is inhibited and associated cancer promoting activity or cancer phenotype is reduced.

26. The method of claim 21, wherein the cytidine deaminase molecule is APOBEC-1.

27. The method of claim 21, wherein the cytidine deaminase activator is an APOBEC-1 activator.

28. The method of claim 26, wherein a selected function of APOBEC-1 is increased such that levels of apoB48 are increased due to cytidine to uridine editing of apoB mRNA and levels of apoB100 are consequently decreased as compared to a control.

29. The method of claim 26, wherein a selected function of APOBEC-1 is decreased by binding to the test compound, and cytidine to uridine editing of mRNA or deoxycytidine to deoxyuridine mutation of DNA is inhibited, and associated cancer promoting activity is reduced.

30. The method of claim 21, wherein a selected function of the cytidine deaminase is promotion of antibody diversity produced by lymphocytes as compared to antibody production by control lymphocytes.

31. The method of claim 25, wherein the cytidine deaminase molecule is AID.

32. The method of claim 31, wherein a selected function of AID is increased such that levels of cytidine to uridine RNA editing or deoxycytidine to deoxyuridine mutation are increased and class switch recombination and somatic hypermuation within the immunoglobulin locus of genes within B lymphocytes is increased.

33. The method of claim 31, wherein a selected function of AID is decreased such that levels of cytidine to uridine RNA editing or deoxycytidine to deoxyuridine mutation are decreased and changes associated with cancer promoting activity are reduced

34. The method of claim 21, wherein the cytidine deaminase activator is an AID activator.

35. The method of claim 22, wherein the viral infectivity is HIV infectivity.

36. The method of claim 22, wherein ability to suppress viral infectivity is measured by contacting the test compound with a cytidine deaminase molecule in the presence of Vif and a virus.

37. A method of making a cytidine deaminase activator comprising:
(a) identifying the cytidine deaminase activator of claim 21; and
(b) modifying the cytidine deaminase activator to enhance the selected deaminase function of the modified cytidine deaminase activator as compared to the function of the unmodified cytidine deaminase activator.

38. A method of making a cytidine deaminase activator comprising:
(a) identifying the cytidine deaminase activator of claim 21; and
(b) modifying the cytidine deaminase activator to lower biotoxicity of the modified cytidine deaminase activator as compared to the biotoxicity of the unmodified cytidine deaminase activator.

39. The method of claim 21, wherein the cytidine deaminase molecule is linked to a reporter.

40. The method of claim 39, wherein the reporter is luciferase.

41. The method of claim 39, wherein the reporter is GFP.

42. The method of claim 39, wherein the reporter is RFP.

43. The method of claim 39, wherein the reporter is FITC.

44. The method of claim 39, wherein the cytidine deaminase molecule and the reporter form a chimera.

45. The method of claim 21, wherein the cytidine deaminase molecule comprises SEQ IDNO: 2.

46. The method of claim 41, wherein the cytidine deaminase molecule has 80% or greater homology to SEQ ID NO: 2.

47. The method of claim 21, wherein a plurality of test compounds are contacted with cytidine deaminase molecules in a high throughput assay system.

48. The method of claim 47, wherein the high throughput assay system comprises an immobilized array of test compounds.

49. The method of claim 47, wherein the high throughput assay system comprises an immobilized array of cytidine deaminase molecules.

50. A cytidine deaminase activator identified by the method of claim 21.

51. A CEM15 activator identified by the method of claim 21.

52. An APOBEC-1 activator identified by the method of claim 21.

53. An AID activator identified by the method of claim 21.

54. A cytidine deaminase activator made by the method of claim 37.

55. A cytidine deaminase activator made by the method of claim 38.

56. A polypeptide comprising 5 or more contiguous amino acid residues of a ubiquitination protein, wherein the polypeptide binds Vif and blocks ubiquitination of CEM15.

57. A polypeptide comprising 5 or more contiguous amino acid residues of a Gag protein, wherein the polypeptide binds CEM15 and promotes CEM15 binding to viral RNA.

58. A method of promoting CEM15 binding to viral RNA comprising contacting CEM15 with the polypeptide of claim 57.

59. A polypeptide comprising 5 or more contiguous amino acid residues of CEM15 wherien the polypeptide binds a ubiquitination protein and blocks Vif-mediated ubiquitination of CEM15.

60. A method of blocking the Vif-mediated ubiquitination of CEM15 comprising contacting the CEM15 with the polypeptide of claim 59.

61. A polypeptide comprising 5 or more contiguous amino acid residues of a CEM15 binding domain on Vif, wherein the polypeptide blocks CEM15-Vif interaction.

62. A method of blocking CEM15-Vif interaction comprising contacting Vif or CEM15 with the polypeptide of claim 61.


SEQUENCE LISTING (70 sequences)

SEQ ID NO: 1 - protein, 384 aa, Artificial Sequence
  Description of Artificial Sequence ; note = synthetic construct
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 2 - DNA, 1155 bp, Artificial Sequence
  Description of Artificial Sequence ; note = synthetic construct
atgaagcctcacttcagaaacacagtggagcgaatgtatcgagacacattctcctacaacttttataatagacccatcctttctcgtcggaataccgtctggctgtgctacgaagtgaaaacaaagggtccctcaaggccccctttggacgcaaagatctttcgaggccaggtgtattccgaacttaagtaccacccagagatgagattcttccactggttcagcaagtggaggaagctgcatcgtgaccaggagtatgaggtcacctggtacatatcctggagcccctgcacaaagtgtacaagggatatggccacgttcctggccgaggacccgaaggttaccctgaccatcttcgttgcccgcctctactacttctgggacccagattaccaggaggcgcttcgcagcctgtgtcagaaaagagacggtccgcgtgccaccatgaagatcatgaattatgacgaatttcagcactgttggagcaagttcgtgtacagccaaagagagctatttgagccttggaataatctgcctaaatattatatattactgcacatcatgctgggggagattctcagacactcgatggatccacccacattcactttcaactttaacaatgaaccttgggtcagaggacggcatgagacttacctgtgttatgaggtggagcgcatgcacaatgacacctgggtcctgctgaaccagcgcaggggctttctatgcaaccaggctccacataaacacggtttccttgaaggccgccatgcagagctgtgcttcctggacgtgattcccttttggaagctggacctggaccaggactacagggttacctgcttcacctcctggagcccctgcttcagctgtgcccaggaaatggctaaattcatttcaaaaaacaaacacgtgagcctgtgcatcttcactgcccgcatctatgatgatcaaggaagatgtcaggaggggctgcgcaccctggccgaggctggggccaaaatttcaataatgacatacagtgaatttaagcactgctgggacacctttgtggaccaccagggatgtcccttccagccctgggatggactagatgagcacagccaagacctgagtgggaggctgcgggccattctccagaatcaggaaaactga

SEQ ID NO: 3 - protein, 198 aa, Artificial Sequence [sequence omitted]

SEQ ID NO: 4 - DNA, 597 bp, Artificial Sequence
  Description of Artificial Sequence ; note = synthetic construct
atggacagcctcttgatgaaccggaggaagtttctttaccaattcaaaaatgtccgctgggctaagggtcggcgtgagacctacctgtgctacgtagtgaagaggcgtgacagtgctacatccttttcactggactttggttatcttcgcaataagaacggctgccacgtggaattgctcttcctccgctacatctcggactgggacctagaccctggccgctgctaccgcgtcacctggttcacctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaagattatttttactgctggaatacttttgtagaaaaccatgaaagaactttcaaagcctgggaagggctgcatgaaaattcagttcgtctctccagacagcttcggcgcatccttttgcccctgtatgaggttgatgacttacgagacgcatttcgtactttgggactttga

SEQ ID NO: 5 - protein, 236 aa, Artificial Sequence
  Description of Artificial Sequence ; note = synthetic construct
MTSEKGPSTGDPTLRRRIEPWEFDVFYDPRELRKEACLLYEIKWGMSRKIWRSSGKNTTNHVEVNFIKKFTSERDFHPSISCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 6 - DNA, 863 bp, Artificial Sequence
  Description of Artificial Sequence ; note = synthetic construct
gatcccagaggaggaagtccagagacagagcaccatgacttctgagaaaggagaagaatcgaaccctgggagtttgacgtcttctatgaccccagagaacttcgtaaagaggcctgtctgctctacgaaatcaagtggggcatgagccggaagatctggcgaagctcaggcaaaaacaccaccaatcacgtggaagttaattttataaaaaaatttacgtcagaaagagattttcacccatccatcagctgctccatcacctggttcttgtcctggagtccctgctgggaatgctcccaggctattagagagtttctgagtcggcaccctggtgtgactctagtgatctacgtagctcggcttttttggcacatggatcaacaaaatcggcaaggtctcagggaccttgttaacagtggagtaactattcagattatgagagcatcagagtattatcactgctggaggaattttgtcaactacccacctggggatgaagctcactggccacaatacccacctctgtggatgatgttgtacgcactggagctgcactgcataattctaagtcttccaccctgtttaaagatttcaagaagatggcaaaatcatcttacatttttcagacttcatcttcaaaactgccattaccaaacgattccgccacacatccttttagctacagggctgatacatccttctgtggcttggagatgaataggatgattccgtgtgtgtactgattcaagaacaagcaatgatgacccactaaagagtgaatgccatttagaatctagaaatgttcacaaggtaccccaaaactctgtagcttaaaccaacaataaatatgtattacctctggc

SEQ ID NO: 7 - protein, 192 aa, Artificial Sequence
  Description of Artificial Sequence ; note = synthetic construct
MENRWQVMIVWQVDRMRIKTWKSLVKHHMYISKKAKEWVYRHHYESTHPRISSEVHIPLGDAKLVITTYWGLHTGEREWHLGQGVSIEWRKKRYNTQVDPDLADKLIHLHYFDCFSDSAIRHAILGHRVRPKCEYQAGHNKVGSLQYLALTALITPKKIKPPLPSVRKLTEDRWNKPQKTKGHRGSHTMNGH

SEQ ID NO: 8 - DNA, 579 bp, Artificial Sequence
  Description of Artificial Sequence ; note = synthetic construct
atggaaaacagatggcaggtgatgattgtgtggcaagtagacaggatgaggattaaaacatggaaaagtttagtaaaacaccatatgtatatttcaaagaaagctaaggaatgggtctatagacatcactatgaaagcactcatccaagaataagttcagaagtacacatcccactaggggatgctaaattagtaataacaacatattggggtctgcatacaggagaaagagaatggcatctgggtcagggagtctccatagaatggaggaaaaagagatataatacacaagtagaccctgacctagcagacaaactaatccacctgcattattttgattgtttttcagactctgctataagacatgccatattaggacatagagttaggcctaagtgtgaatatcaagcaggacataacaaggtagggtctctacagtacttggcactaacagcattaataacaccaaaaaagataaagccacctttgcctagtgttaggaaactaacagaggatagatggaacaagccccagaagaccaagggccacagagggagccatacaatgaatggacactag

SEQ ID NO: 9 - protein, 4 aa, Artificial Sequence
  Description of Artificial Sequence ; note = synthetic construct
RGYW

SEQ ID NO: 10 - DNA, 20 bp, Artificial Sequence
  Description of Artificial Sequence ; note = synthetic construct
cactttagggagggctgtcc

SEQ ID NO: 11 - DNA, 20 bp, Artificial Sequence
  Description of Artificial Sequence ; note = synthetic construct
ctgtgatcagctggagatgg

SEQ ID NO: 12 - DNA, 33 bp, Artificial Sequence
  Description of Artificial Sequence ; note = synthetic construct
ctcccatggcaaagcctcacttcagaaacacag

SEQ ID NO: 13 - DNA, 35 bp, Artificial Sequence
  Description of Artificial Sequence ; note = synthetic construct
ctcctcgaggttttcctgattctggagaatggccc

SEQ ID NO: 14 - protein, 162 aa, Artificial Sequence
  Description of Artificial Sequence ; note = synthetic construct
LRRRIEPWEFDVFYDPRELRKEACLLYEIKWGMSRKIWRSSGKNTTNHVEVNFIKKFTSERDFHPSISCSITWFLSWSPCWECSQAIREFLSRHPGVTLVIYVARLFWHMDQQNRQGLRDLVNSGVTIQIMRASEYYHCWRNFVNYPPGDEAHWPQYPPLWM

SEQ ID NO: 15 - protein, 171 aa, Artificial Sequence
  Description of Artificial Sequence ; note = synthetic construct
IRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFR

SEQ ID NO: 16 - protein, 156 aa, Artificial Sequence
  Description of Artificial Sequence ; note = synthetic construct
RRIEPWEFDVFYDPRELRKEACLLYEIKWGMSRKIWRSSGKNTTNHVEVNFIKKFTSERDFHPSISCSITWFLSWSPCWECSQAIREFLSRHPGVTLVIYVARLFWHMDQQNRQGLRDLVNSGVTIQIMRASEYYHCWRNFVNYPPGDEAHWPQYP

SEQ ID NO: 17 - protein, 163 aa, Artificial Sequence
  Description of Artificial Sequence ; note = synthetic construct
RRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFWPWKGLE

SEQ ID NO: 18 - protein, 162 aa, Artificial Sequence; identical to SEQ ID NO: 14
  Description of Artificial Sequence ; note = synthetic construct

SEQ ID NO: 19 - protein, 171 aa, Artificial Sequence
  Description of Artificial Sequence ; note = synthetic construct
IRNLISQETFKFHFKNLRYAIDRKDTFLCYEVTRKDCDSPVSLHHGVFKNKDNIHAEICFLYWFHDKVLKVLSPREEFKITWYMSWSPCFECAEQVLRFLATHHNLSLDIFSSRLYNIRDPENQQNLCRLVQEGAQVAAMDLYEFKKCWKKFVDNGGRRFRPWKKLLTNFR

SEQ ID NO: 20 - protein, 156 aa, Artificial Sequence; identical to SEQ ID NO: 16
  Description of Artificial Sequence ; note = synthetic construct

SEQ ID NO: 21 - protein, 160 aa, Artificial Sequence
  Description of Artificial Sequence ; note = synthetic construct
HLLSEEEFYSQFYNQRVKHLCYYHGMKPYLCYQLEQFNGQAPLKGCLLSEKGKQHAEILFLDKIRSMELSQVIITCYLTWSPCPNCAWQLAAFKRDRPDLILHIYTSRLYFHWKRPFQKGLCSLWQSGILVDVMDLPQFTDCWTNFVNPKRPFWPWKGLE

SEQ ID NO: 22 - DNA, 621 bp, Artificial Sequence
  Description of Artificial Sequence ; note = synthetic construct
agtcctggggtctgcaagatttggtgaatgactttggaaacctacagcttggacccccgatgtcttgagaggcaagaagagattcaagaaggtcttttggtgacccccccacccaaccccaagtctaggagaccttttgttctcccgtttgtttccccttttgttttatcttttgttgttttgctttgttttgaagacagagtctcactgggtagcttgctactctggaactcactactagactaagctggccttaaactctaaaatccacctgccaatgccttctgagagccaggcttaaggtgtgcgctgcccactcccagccttaacccactgtggcttttccttcctctttcttttattatctttttatctcccctcaccctcccgccatcaataggtacttaattttgtacttgaaatttttaagttgggccaggcatggtggagcagcgtgcctctaatcgcaggcaggaggatttccacgagcttgaggctagcctgatctacatagtgggctccaggacagccagaactacacagagaccctgtctcaaaaataaatttagatagataaatacataaataaataaatggaagaagtcaaagaaagaaagacaa

SEQ ID NO: 23 - DNA, 596 bp, Artificial Sequence
  Description of Artificial Sequence ; note = synthetic construct
agtcctggggtctgcaagatttggtgaatgactttggaaacctacagcttggacccccgatgtcttgagaggcaagaagagattcaagaaggtcttttggtgacccccccacccaaccccaagtctaggagaccttttgttctcctgtttgtttccccttttgttttatcttttgttgttttgctttgttttgaagacagagtctcactgggtagcttgctactctggaactcactactagactaagctggccttaaactctaaaatccacctgccagtgccttctgagagccaggcttaaggtgtgcgctgcccactcccagccttaacccactgtggcttttccttcctctttcttttattatctttttatctcccctcaccctcccgccatcaataggtacttaattttgtacttgaaatttttaagttgggccaggcatggtggagcagcgtgcctctaatcgcaggcaggaggatttccacgagcttgaggctagcctgatctacatagtgggctccaggacagccagaactacacagagaccctgtctcaaaaataaatttagatagataaatacataaataaatggaag

SEQ ID NO: 24 - DNA, 279 bp, Artificial Sequence
  Description of Artificial Sequence ; note = synthetic construct
aggacaacatccacgctgaaatctgctttttatactggttccatgacaaagtactgaaagtgctgtctccgagagaagagttcaagatcacctggtatatgtcctggagcccctgtttcgaatgtgcagagcaggtactaaggttcctggctacacaccacaacctgagcctggacatcttcagctcccgcctctacaacatacgggacccagaaaaccagcagaatctttgcaggctggttcaggaaggagcccaggtggctgccatggacctatacg

SEQ ID NO: 25 - DNA, 279 bp, Artificial Sequence
  Description of Artificial Sequence ; note = synthetic construct
aggacaacatccacgctgaaatctgctttttatactggttccatgacaaagtactgaaagtgctgtctccgagagaagagttcaagatcacctggtatatgtcctggagcccctgtttcgaatgtgcagagcagatagtaaggttcctggctacacaccacaacctgagcctggacatcttcagctcccgcctctacaacgtacaggacccagaaacccagcagaatctttgcaggctggttcaggaaggagcccaggtggctgccatggacctatacg

SEQ ID NO: 26 - DNA, 264 bp, Artificial Sequence
  Description of Artificial Sequence ; note = synthetic construct
agaaaggcaaacagcatgcagaaatcctcttccttgataagattcggtccatggagctgagccaagtgataatcacctgctacctcacctggagcccctgcccaaactgtgcctggcaactggcggcattcaaaagggatcgtccagatctaattctgcatatctacacctcccgcctgtatttccactggaagaggcccttccagaaggggctgtgttctctgtggcaatcagggatcctggtggacgtcatggacctcccac

SEQ ID NO: 27 - DNA, 204 bp, Artificial Sequence
  Description of Artificial Sequence ; note = synthetic construct
agaaaggcaaacagcatgcagaaatcctcttccttgataagattcggtccatggagctgagccaagtgacaatcacctgctacctcacctggagcccctgcccaaactgtgcctggcaacatttccactggaagaggcccttccagaaggggctgtgttctctgtggcaatcagggatcctggtggacgtcatggacctcccac

SEQ ID NO: 28 - DNA, 159 bp, Artificial Sequence
  Description of Artificial Sequence ; note = synthetic construct
aggcgagtgcacctgctaagtgaagaggaattttactcgcagttttacaaccaacgagtcaagcatctctgctactaccacggcatgaagccctatctatgctaccagctggagcagttcaatggccaagcgccactcaaaggctgcctgctaagcgag

SEQ ID NO: 29 - DNA, 159 bp, Artificial Sequence
  Description of Artificial Sequence ; note = synthetic construct
aggcgaatggacccgctaagtgaagaggaattttactcgcagttttacaaccaacgagtcaagcatctctgctactaccaccgcatgaagccctatctatgctaccagctggagcagttcaatggccaagcgccactcaaaggctgcctgctaagcgag

SEQ ID NO: 30 - DNA, 268 bp, Artificial Sequence
  Description of Artificial Sequence ; note = synthetic construct
cagaaacctgatatctcaagaaacattcaaattccactttaagaacctacgctatgccatagaccggaaagataccttcttgtgctatgaagtgactagaaaggactgcgattcacccgtctcccttcaccatggggtctttaagaacaagggaatttaaaaagtgttggaagaagtttgtggacaatggcggcaggcgattcaggccttggaaaaaactgcttacaaattttagataccaggattctaagcttcaggagattctgag

SEQ ID NO: 31 - DNA, 268 bp, Artificial Sequence
  Description of Artificial Sequence ; note = synthetic construct
cagaaacctgatatctcaagaaacattcaagttccactttaagaacctaggctatgccaaaggccggaaagataccttcttgtgctatgaagtgactagaaaggactgcgattcacccgtctcccttcaccatggggtctttaagaacaagggaatttaaaaagtgttggaagaagtttgtggacaatggtggcaggcgattcaggccttggaaaagactgcttacaaattttagataccaggattctaagcttcaggagattctgag

SEQ ID NO: 32 - DNA, 219 bp, Artificial Sequence
  Description of Artificial Sequence ; note = synthetic construct
agtttactgactgctggacaaactttgtgaacccgaaaaggccgttttggccatggaaaggattggagataatcagcaggcgcacacaaaggcggctccacaggatcaaggagagaccttgctacatcccggtcccttccagctcttcatccactctgtcaaatatctgtctaacaaaaggtctcccagagacgaggttctgcgtggagggcaggcgag

SEQ ID NO: 33 - DNA, 332 bp, Artificial Sequence
  Description of Artificial Sequence ; note = synthetic construct
agtttactgactgctggacaaactttgtgaacccgaaaaggccgttttggccatggaaaggattggagataatcagcaggcgcacacaaaggcggctccacaggatcaaggaggattggagataatcagcaggcgcacacaaaggcggctccgcaggatcaaggagagaccttgctacatcccggtcccttccagctcttcatccactctgtcaaatatctgtctaagaccttgctacatctcggtcccttccagctcttcatccactctgtcaaatatctgtctaacaaaaggtctcccagagacgaggttctgcgtggagggcaggcgag

SEQ ID NO: 34 - DNA, 53 bp, Artificial Sequence
  Description of Artificial Sequence ; note = synthetic construct
atgggaccattctgtctgggatgcagccatcgcaaatgctattcaccgatcag

SEQ ID NO: 35 - DNA, 53 bp, Artificial Sequence; identical to SEQ ID NO: 34
  Description of Artificial Sequence ; note = synthetic construct

SEQ ID NO: 36 - RNA, 4 nt, Artificial Sequence
  Description of Artificial Sequence ; note = synthetic construct
ugau

SEQ ID NO: 37 - DNA, 20 bp, Artificial Sequence
  Description of Artificial Sequence ; note = synthetic construct
ttacctgggtctatggcagt

SEQ ID NO: 38 - DNA, 19 bp, Artificial Sequence
  Description of Artificial Sequence ; note = synthetic construct
tgaaggctcagaatccccc

SEQ ID NO: 39 - protein, 738 aa, Artificial Sequence
  Description of Artificial Sequence ; note = synthetic construct
MRKKRRQRRRVDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLSRQLRRILLPLYEVDDLRDAFRTLGLHAAMADTFLEHMCRLDIDSEPTIARNTGIICTIGPASRSVDKLKEMIKSGMNVARLNFSHGTHEYHEGTIKNVREATESFASDPITYRPVAIALDTKGPEIRTGLIKGSGTAEVELKKGAALKVTLDNAFMENCDENVLWVDYKNLIKVIDVGSKIYVDDGLISLLVKEKGKDFVMTEVENGGMLGSKKGVNLPGAAVDLPAVSEKDIQDLKFGVEQNVDMVFASFIRKAADVHAVRKVLGEKGKHIKIISKIENHEGVRRFDEIMEASDGIMVARGDLGIEIPAEKVFLAQKMMIGRCNRAGKPIICATQMLESMIKKPRPTRAEGSDVANAVLDGADCIMLSGETAKGDYPLEAVRMQHAIAREAEAAMFHRQQFEEILRHSVHHREPADAMAAGAVEASFKCLAAALIVMTESGRSAHLVSRYRPRAPIIAVTRNDQTARQAHLYRGVFPVLCKQPAHDAWAEDVDLRVNLGMNVGKARGFFKTGDLVIVLTGWRPGSGYTNTMRVVPVPLEYPYDVPDYAHHHHHH

SEQ ID NO: 40 - DNA, 2217 bp, Artificial Sequence
  Description of Artificial Sequence ; note = synthetic construct
atgagaaaaaaaagaagacaaagaagaagagtggacagcctcttgatgaaccggaggaagtttctttaccaattcaaaaatgtccgctgggctaagggtcggcgtgagacctacctgtgctacgtagtgaagaggcgtgacagtgctacatccttttcactggactttggttatcttcgcaataagaacggctgccacgtggaattgctcttcctccgctacatctcggactgggacctagaccctggccgctgctaccgcgtcacctggttcacctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaagattatttttactgctggaatacttttgtagaaaaccatgaaagaactttcaaagcctgggaagggctgcatgaaaattcagttcgtctctccagacagcttcgacgaatccttttgcccctgtatgaggttgatgacttacgagacgcatttcgtactttgggacttcacgctgccatggcagacacctttctggagcacatgtgccgcctggacatcgactccgagccaaccattgccagaaacaccggcatcatctgcaccatcggcccagcctcccgctctgtggacaagctgaaggaaatgattaaatctggaatgaatgttgcccgcctcaacttctcgcacggcacccacgagtatcatgagggcacaattaagaacgtgcgagaggccacagagagctttgcctctgacccgatcacctacagacctgtggctattgcactggacaccaagggacctgaaatccgaactggactcatcaagggaagtggcacagcagaggtggagctcaagaagggcgcagctctcaaagtgacgctggacaatgccttcatggagaactgcgatgagaatgtgctgtgggtggactacaagaacctcatcaaagttatagatgtgggcagcaaaatctatgtggatgacggtctcatttccttgctggttaaggagaaaggcaaggactttgtcatgactgaggttgagaacggtggcatgcttggtagtaagaagggagtgaacctcccaggtgctgcggtcgacctgcctgcagtctcagagaaggacattcaggacctgaaatttggcgtggagcagaatgtggacatggtgttcgcttccttcatccgcaaagctgctgatgtccatgctgtcaggaaggtgctaggggaaaagggaaagcacatcaagattatcagcaagattgagaatcacgagggtgtgcgcaggtttgatgagatcatggaggccagcgatggcattatggtggcccgtggtgacctgggtattgagatccctgctgaaaaagtcttcctcgcacagaagatgatgattgggcgctgcaacagggctggcaaacccatcatttgtgccactcagatgttggaaagcatgatcaagaaacctcgcccgacccgcgctgagggcagtgatgttgccaatgcagttctggatggagcagactgcatcatgctgtctggggagaccgccaagggagactacccactggaggctgtgcgcatgcagcacgctattgctcgtgaggctgaggccgcaatgttccatcgtcagcagtttgaagaaatcttacgccacagtgtacaccacagggagcctgctgatgccatggcagcaggcgcggtggaggcctcctttaagtgcttagcagcagctctgatagttatgaccgagtctggcaggtctgcacacctggtgtcccggtaccgcccgcgggctcccatcatcgccgtcacccgcaatgaccaaacagcacgccaggcacacctgtaccgcggcgtcttccccgtgctgtgcaagcagccggcccacgatgcctgggcagaggatgtggatctccgtgtgaacctgggcatgaatgtcggcaaagcccgtggattcttcaagaccggggacctggtgatcgtgctgacgggctggcgccccggctccggctacaccaacaccatgcgggtggtgcccgtgccactcgagtacccctacgacgtgcccgactacgcccaccaccaccaccaccactga

SEQ ID NO: 41 - protein, 530 aa, Artificial Sequence
  Description of Artificial Sequence ; note = synthetic construct
MSKHHDAGTAFIQTQQLHAAMADTFLEHMCRLDIDSEPTIARNTGIICTIGPASRSVDKLKEMIKSGMNVARLNFSHGTHEYHEGTIKNVREATESFASDPITYRPVAIALDTKGPEIRTGLIKGSGTAEVELKKGAALKVTLDNAFMENCDENVLWVDYKNLIKVIDVGSKIYVDDGLISLLVKEKGKDFVMTEVENGGMLGSKKGVNLPGAAVDLPAVSEKDIQDLKFGVEQNVDMVFASFIRKAADVHAVRKVLGEKGKHIKIISKIENHEGVRRFDEIMEASDGIMVARGDLGIEIPAEKVFLAQKMMIGRCNRAGKPIICATQMLESMIKKPRPTRAEGSDVANAVLDGADCIMLSGETAKGDYPLEAVRMQHAIAREAEAAMFHRQQFEEILRHSVHHREPADAMAAGAVEASFKCLAAALIVMTESGRSAHLVSRYRPRAPIIAVTRNDQTARQAHLYRGVFPVLCKQPAHDAWAEDVDLRVNLGMNVGKARGFFKTGDLVIVLTGWRPGSGYTNTMRVVPVP

SEQ ID NO: 42 - DNA, 1593 bp, Artificial Sequence
  Description of Artificial Sequence ; note = synthetic construct
atgtcgaagcaccacgatgcagggaccgctttcatccagacccagcagctgcacgctgccatggcagacacctttctggagcacatgtgccgcctggacatcgactccgagccaaccattgccagaaacaccggcatcatctgcaccatcggcccagcctcccgctctgtggacaagctgaaggaaatgattaaatctggaatgaatgttgcccgcctcaacttctcgcacggcacccacgagtatcatgagggcacaattaagaacgtgcgagaggccacagagagctttgcctctgacccgatcacctacagacctgtggctattgcactggacaccaagggacctgaaatccgaactggactcatcaagggaagtggcacagcagaggtggagctcaagaagggcgcagctctcaaagtgacgctggacaatgccttcatggagaactgcgatgagaatgtgctgtgggtggactacaagaacctcatcaaagttatagatgtgggcagcaaaatctatgtggatgacggtctcatttccttgctggttaaggagaaaggcaaggactttgtcatgactgaggttgagaacggtggcatgcttggtagtaagaagggagtgaacctcccaggtgctgcggtcgacctgcctgcagtctcagagaaggacattcaggacctgaaatttggcgtggagcagaatgtggacatggtgttcgcttccttcatccgcaaagctgctgatgtccatgctgtcaggaaggtgctaggggaaaagggaaagcacatcaagattatcagcaagattgagaatcacgagggtgtgcgcaggtttgatgagatcatggaggccagcgatggcattatggtggcccgtggtgacctgggtattgagatccctgctgaaaaagtcttcctcgcacagaagatgatgattgggcgctgcaacagggctggcaaacccatcatttgtgccactcagatgttggaaagcatgatcaagaaacctcgcccgacccgcgctgagggcagtgatgttgccaatgcagttctggatggagcagactgcatcatgctgtctggggagaccgccaagggagactacccactggaggctgtgcgcatgcagcacgctattgctcgtgaggctgaggccgcaatgttccatcgtcagcagtttgaagaaatcttacgccacagtgtacaccacagggagcctgctgatgccatggcagcaggcgcggtggaggcctcctttaagtgcttagcagcagctctgatagttatgaccgagtctggcaggtctgcacacctggtgtcccggtaccgcccgcgggctcccatcatcgccgtcacccgcaatgaccaaacagcacgccaggcacacctgtaccgcggcgtcttccccgtgctgtgcaagcagccggcccacgatgcctgggcagaggatgtggatctccgtgtgaacctgggcatgaatgtcggcaaagcccgtggattcttcaagaccggggacctggtgatcgtgctgacgggctggcgccccggctccggctacaccaacaccatgcgggtggtgcccgtgccatga

SEQ ID NO: 43 - protein, 9 aa, Artificial Sequence
  Description of Artificial Sequence ; note = synthetic construct
RKKRRQRRR

SEQ ID NO: 44 - DNA, 27 bp, Artificial Sequence
  Description of Artificial Sequence ; note = synthetic construct
agaaaaaaaagaagacaaagaagaaga

SEQ ID NO: 45 - protein, 237 aa, Artificial Sequence
  Description of Artificial Sequence ; note = synthetic construct
MTSEKGPSTGDPTLRRRIEPWEFDVFYDPRELRKEACLLYEIKWGMSRKIWRSSGKNTTNHVEVNFIKKFTSERDFHPSISCSITWFLSWSPCWECSQAIREFLSRHPGVTLVILYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 46 - protein, 9 aa, Artificial Sequence
  Description of Artificial Sequence ; note = synthetic construct
YPYDVPDYA

SEQ ID NO: 47 - DNA, 27 bp, Artificial Sequence
  Description of Artificial Sequence ; note = synthetic construct
tacccctacgacgtgcccgactacgcc

SEQ ID NO: 48 - protein, 429 aa, Artificial Sequence
  Description of Artificial Sequence ; note = synthetic construct
MGPFCLGCSHRKCYSPIRNLISQETFKFHFKNLRYAIDRKDTFLCYEVTRKDCDSPVSLHHGVFKNKDNIHAEICFLYWFHDKVLKVLSPREEFKITWYMSWSPCFECAEQVLRFLATHHNLSLDIFSSRLYNIRDPENQQNLCRLVQEGAQVAAMDLYEFKKCWKKFVDNGGRRFRPWKKLLTNFRYQDSKLQEILRPCYIPVPSSSSSTLSNICLTKGLPETRFCVEGRRVHLLSEEEFYSQFYNQRVKHLCYYHGMKPYLCYQLEQFNGQAPLKGCLLSEKGKQHAEILFLDKIRSMELSQVIITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLHRIKESWGLQDLVNDFGNLQLGPPMS

SEQ ID NO: 49 - DNA, 1948 bp, Artificial Sequence
  Description of Artificial Sequence ; note = synthetic construct
acttggcccgggaggtcagtttcacttctgggggtcttccatagcctgctcacagaaaatgcaaccccagcgcatggggcccagagctgggatgggaccattctgtctgggatgcagccatcgcaaatgctattcaccgatcagaaacctgatatctcaagaaacattcaaattccactttaagaacctacgctatgccatagaccggaaagataccttcttgtgctatgaagtgactagaaaggactgcgattcacccgtctcccttcaccatggggtctttaagaacaaggacaacatccacgctgaaatctgctttttatactggttccatgacaaagtactgaaagtgctgtctccgagagaagagttcaagatcacctggtatatgtcctggagcccctgtttcgaatgtgcagagcaggtactaaggttcctggctacacaccacaacctgagcctggacatcttcagctcccgcctctacaacatacgggacccagaaaaccagcagaatctttgcaggctggttcaggaaggagcccaggtggctgccatggacctatacgaatttaaaaagtgttggaagaagtttgtggacaatggcggcaggcgattcaggccttggaaaaaactgcttacaaattttagataccaggattctaagcttcaggagattctgagaccttgctacatcccggtcccttccagctcttcatccactctgtcaaatatctgtctaacaaaaggtctcccagagacgaggttctgcgtggagggcaggcgagtgcacctgctaagtgaagaggaattttactcgcagttttacaaccaacgagtcaagcatctctgctactaccacggcatgaagccctatctatgctaccagctggagcagttcaatggccaagcgccactcaaaggctgcctgctaagcgagaaaggcaaacagcatgcagaaatcctcttccttgataagattcggtccatggagctgagccaagtgataatcacctgctacctcacctggagcccctgcccaaactgtgcctggcaactggcggcattcaaaagggatcgtccagatctaattctgcatatctacacctcccgcctgtatttccactggaagaggcccttccagaaggggctgtgttctctgtggcaatcagggatcctggtggacgtcatggacctcccacagtttactgactgctggacaaactttgtgaacccgaaaaggccgttttggccatggaaaggattggagataatcagcaggcgcacacaaaggcggctccacaggatcaaggagtcctggggtctgcaagatttggtgaatgactttggaaacctacagcttggacccccgatgtcttgagaggcaagaagagattcaagaaggtcttttggtgacccccccacccaaccccaagtctaggagaccttttgttctcccgtttgtttccccttttgttttatcttttgttgttttgctttgttttgaagacagagtctcactgggtagcttgctactctggaactcactactagactaagctggccttaaactctaaaatccacctgccaatgccttctgagagccaggcttaaggtgtgcgctgcccactcccagccttaacccactgtggcttttccttcctctttcttttattatctttttatctcccctcaccctcccgccatcaataggtacttaattttgtacttgaaatttttaagttgggccaggcatggtggagcagcgtgcctctaatcgcaggcaggaggatttccacgagcttgaggctagcctgatctacatagtgggctccaggacagccagaactacacagagaccctgtctcaaaaataaatttagatagataaatacataaataaataaatggaagaagtcaaagaaagaaagacaaaaaaaaaaaaaaaa

SEQ ID NO: 50 - protein, 7 aa, Artificial Sequence
  Description of Artificial Sequence ; note = synthetic construct
ENLYFQG

SEQ ID NO: 51 - DNA, 21 bp, Artificial Sequence
  Description of Artificial Sequence ; note = synthetic construct
gagaatctgtattttcaaggt

SEQ ID NO: 52 - protein, 239 aa, Artificial Sequence
  Description of Artificial Sequence ; note = synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 53 - DNA, 720 bp, Artificial Sequence
  Description of Artificial Sequence ; note = synthetic construct
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 54 - protein, 209 aa, Artificial Sequence
  Description of Artificial Sequence ; note = synthetic construct
MENRWQVMIVWQVDRMRIKTWKSLVKHHMYISKKAKEWVYRHHYESTHPRISSEVHIPLGDAKLVITTYWGLHTGEREWHLGQGVSIEWRKKRYNTQVDPDLADKLIHLHYFDCFSDSAIRHAILGHRVRPKCEYQAGHNKVGSLQYLALTALITPKKIKPPLPSVRKLTEDRWNKPQKTKGHRGSHTMNGHGYPYDVPDYAGHHHHHH

SEQ ID NO: 55 - DNA, 630 bp, Artificial Sequence
  Description of Artificial Sequence ; note = synthetic construct
atggaaaacagatggcaggtgatgattgtgtggcaagtagacaggatgaggattaaaacatggaaaagtttagtaaaacaccatatgtatatttcaaagaaagctaaggaatgggtctatagacatcactatgaaagcactcatccaagaataagttcagaagtacacatcccactaggggatgctaaattagtaataacaacatattggggtctgcatacaggagaaagagaatggcatctgggtcagggagtctccatagaatggaggaaaaagagatataatacacaagtagaccctgacctagcagacaaactaatccacctgcattattttgattgtttttcagactctgctataagacatgccatattaggacatagagttaggcctaagtgtgaatatcaagcaggacataacaaggtagggtctctacagtacttggcactaacagcattaataacaccaaaaaagataaagccacctttgcctagtgttaggaaactaacagaggatagatggaacaagccccagaagaccaagggccacagagggagccatacaatgaatggacacggttacccctacgacgtgcccgactacgccggtcaccaccaccatcatcattga

SEQ ID NO: 56 - protein, 454 aa, Artificial Sequence
  Description of Artificial Sequence ; note = synthetic construct
MENRWQVMIVWQVDRMRIKTWKSLVKHHMYISKKAKEWVYRHHYESTHPRISSEVHIPLGDAKLVITTYWGLHTGEREWHLGQGVSIEWRKKRYNTQVDPDLADKLIHLHYFDCFSDSAIRHAILGHRVRPKCEYQAGHNKVGSLQYLALTALITPKKIKPPLPSVRKLTEDRWNKPQKTKGHRGSHTMNGHGENLYFQGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKYPYDVPDYAHHHHHH

SEQ ID NO: 57 - DNA, 600 bp, Artificial Sequence
  Description of Artificial Sequence ; note = synthetic construct
atggaaaacagatggcaggtgatgattgtgtggcaagtagacaggatgaggattaaaacatggaaaagtttagtaaaacaccatatgtatatttcaaagaaagctaaggaatgggtctatagacatcactatgaaagcactcatccaagaataagttcagaagtacacatcccactaggggatgctaaattagtaataacaacatattggggtctgcatacaggagaaagagaatggcatctgggtcagggagtctccatagaatggaggaaaaagagatataatacacaagtagaccctgacctagcagacaaactaatccacctgcattattttgattgtttttcagactctgctataagacatgccatattaggacatagagttaggcctaagtgtgaatatcaagcaggacataacaaggtagggtctctacagtacttggcactaacagcattaataacaccaaaaaagataaagccacctttgcctagtgttaggaaactaacagaggatagatggaacaagccccagaagaccaagggccacagagggagccatacaatgaatggacacggtgagaatctgtattttcaaggt

SEQ ID NO: 58 - protein, 748 aa, Artificial Sequence
  Description of Artificial Sequence ; note = synthetic construct
MENRWQVMIVWQVDRMRIKTWKSLVKHHMYISKKAKEWVYRHHYESTHPRISSEVHIPLGDAKLVITTYWGLHTGEREWHLGQGVSIEWRKKRYNTQVDPDLADKLIHLHYFDCFSDSAIRHAILGHRVRPKCEYQAGHNKVGSLQYLALTALITPKKIKPPLPSVRKLTEDRWNKPQKTKGHRGSHTMNGHGENLYFQGMSKHHDAGTAFIQTQQLHAAMADTFLEHMCRLDIDSEPTIARNTGIICTIGPASRSVDKLKEMIKSGMNVARLNFSHGTHEYHEGTIKNVREATESFASDPITYRPVAIALDTKGPEIRTGLIKGSGTAEVELKKGAALKVTLDNAFMENCDENVLWVDYKNLIKVIDVGSKIYVDDGLISLLVKEKGKDFVMTEVENGGMLGSKKGVNLPGAAVDLPAVSEKDIQDLKFGVEQNVDMVFASFIRKAADVHAVRKVLGEKGKHIKIISKIENHEGVRRFDEIMEASDGIMVARGDLGIEIPAEKVFLAQKMMIGRCNRAGKPIICATQMLESMIKKPRPTRAEGSDVANAVLDGADCIMLSGETAKGDYPLEAVRMQHAIAREAEAAMFHRQQFEEILRHSVHHREPADAMAAGAVEASFKCLAAALIVMTESGRSAHLVSRYRPRAPIIAVTRNDQTARQAHLYRGVFPVLCKQPAHDAWAEDVDLRVNLGMNVGKARGFFKTGDLVIVLTGWRPGSGYTNTMRVVPVPGYPYDVPDYAIEHHHHHH

SEQ ID NO: 59 - DNA, 2247 bp, Artificial Sequence
  Description of Artificial Sequence ; note = synthetic construct
atggaaaacagatggcaggtgatgattgtgtggcaagtagacaggatgaggattaaaacatggaaaagtttagtaaaacaccatatgtatatttcaaagaaagctaaggaatgggtctatagacatcactatgaaagcactcatccaagaataagttcagaagtacacatcccactaggggatgctaaattagtaataacaacatattggggtctgcatacaggagaaagagaatggcatctgggtcagggagtctccatagaatggaggaaaaagagatataatacacaagtagaccctgacctagcagacaaactaatccacctgcattattttgattgtttttcagactctgctataagacatgccatattaggacatagagttaggcctaagtgtgaatatcaagcaggacataacaaggtagggtctctacagtacttggcactaacagcattaataacaccaaaaaagataaagccacctttgcctagtgttaggaaactaacagaggatagatggaacaagccccagaagaccaagggccacagagggagccatacaatgaatggacacggtgagaatctgtattttcaaggtatgtcgaagcaccacgatgcagggaccgctttcatccagacccagcagctgcacgctgccatggcagacacctttctggagcacatgtgccgcctggacatcgactccgagccaaccattgccagaaacaccggcatcatctgcaccatcggcccagcctcccgctctgtggacaagctgaaggaaatgattaaatctggaatgaatgttgcccgcctcaacttctcgcacggcacccacgagtatcatgagggcacaattaagaacgtgcgagaggccacagagagctttgcctctgacccgatcacctacagacctgtggctattgcactggacaccaagggacctgaaatccgaactggactcatcaagggaagtggcacagcagaggtggagctcaagaagggcgcagctctcaaagtgacgctggacaatgccttcatggagaactgcgatgagaatgtgctgtgggtggactacaagaacctcatcaaagttatagatgtgggcagcaaaatctatgtggatgacggtctcatttccttgctggttaaggagaaaggcaaggactttgtcatgactgaggttgagaacggtggcatgcttggtagtaagaagggagtgaacctcccaggtgctgcggtcgacctgcctgcagtctcagagaaggacattcaggacctgaaatttggcgtggagcagaatgtggacatggtgttcgcttccttcatccgcaaagctgctgatgtccatgctgtcaggaaggtgctaggggaaaagggaaagcacatcaagattatcagcaagattgagaatcacgagggtgtgcgcaggtttgatgagatcatggaggccagcgatggcattatggtggcccgtggtgacctgggtattgagatccctgctgaaaaagtcttcctcgcacagaagatgatgattgggcgctgcaacagggctggcaaacccatcatttgtgccactcagatgttggaaagcatgatcaagaaacctcgcccgacccgcgctgagggcagtgatgttgccaatgcagttctggatggagcagactgcatcatgctgtctggggagaccgccaagggagactacccactggaggctgtgcgcatgcagcacgctattgctcgtgaggctgaggccgcaatgttccatcgtcagcagtttgaagaaatcttacgccacagtgtacaccacagggagcctgctgatgccatggcagcaggcgcggtggaggcctcctttaagtgcttagcagcagctctgatagttatgaccgagtctggcaggtctgcacacctggtgtcccggtaccgcccgcgggctcccatcatcgccgtcacccgcaatgaccaaacagcacgccaggcacacctgtaccgcggcgtcttccccgtgctgtgcaagcagccggcccacgatgcctgggcagaggatgtggatctccgtgtgaacctgggcatgaatgtcggcaaagcccgtggattcttcaagaccggggacctggtgatcgtgctgacgggctggcgccccggctccggctacaccaacaccatgcgggtggtgcccgtgccaggttatccgtatgatgtgccagattatgccatcgagcaccaccaccaccaccactga

SEQ ID NO: 60 - protein, 38 aa, Artificial Sequence
  Description of Artificial Sequence; note = synthetic construct
HXEXXFXXXXXXXXXXXXXXXXXXXXXXXXSWXPCXXC

SEQ ID NO: 61 - protein, 30 aa, Artificial Sequence
  Description of Artificial Sequence; note = synthetic construct
XXEXXFXXXXXXXXXXXXXXXXXXXPCXXC

SEQ ID NO: 62 - protein, 43 aa, Artificial Sequence
  Description of Artificial Sequence; note = synthetic construct
XXEXXFXXXXXXXXXXXXXXXXXXXXXXXXXXXXXXPCXXXXC

SEQ ID NO: 63 - DNA, 12 bp, Artificial Sequence
  Description of Artificial Sequence; note = synthetic construct
tagtaacccggg

SEQ ID NO: 64 - DNA, 4 bp, Artificial Sequence
  Description of Artificial Sequence; note = synthetic construct
gact

SEQ ID NO: 65 - DNA, 4 bp, Artificial Sequence
  Description of Artificial Sequence; note = synthetic construct
gaag

SEQ ID NO: 66 - protein, 12 aa, Artificial Sequence
  Description of Artificial Sequence; note = synthetic construct
SNQGGSPLPRSV

SEQ ID NO: 67 - protein, 12 aa, Artificial Sequence
  Description of Artificial Sequence; note = synthetic construct
LPLPAPSFHRTT

SEQ ID NO: 68 - protein, 16 aa, Artificial Sequence
  Description of Artificial Sequence; note = synthetic construct
RQIKIWFQNRRMKWKK

SEQ ID NO: 69 - protein, 12 aa, Artificial Sequence
  Description of Artificial Sequence; note = synthetic construct
YGRKKRRQRRRG

SEQ ID NO: 70 - protein, 13 aa, Artificial Sequence
  Description of Artificial Sequence; note = synthetic construct
DLGEQHFKGLVLI